SEQUENCE LISTING (22 sequences)

SEQ ID NO: 1 - DNA, 1302 bp, Homo sapiens
atgtgtttttctcccattctggaaatcaacatgcagtctgaatctaacattacagtgcgagatgacattgatgacatcaacaccaatatgtaccaaccactatcatatccgttaagctttcaagtgtctctcaccggatttcttatgttagaaattgtgttgggacttggcagcaacctcactgtattggtactttactgcatgaaatccaacttaatcaactctgtcagtaacattattacaatgaatcttcatgtacttgatgtaataatttgtgtgggatgtattcctctaactatagttatccttctgctttcactggagagtaacactgctctcatttgctgtttccatgaggcttgtgtatcttttgcaagtgtctcaacagcaatcaacgtttttgctatcactttggacagatatgacatctctgtaaaacctgcaaaccgaattctgacaatgggcagagctgtaatgttaatgatatccatttggattttttcttttttctctttcctgattccttttattgaggtaaattttttcagtcttcaaagtggaaatacctgggaaaacaagacacttttatgtgtcagtacaaatgaatactacactgaactgggaatgtattatcacctgttagtacagatcccaatattctttttcactgttgtagtaatgttaatcacatacaccaaaatacttcaggctcttaatattcgaataggcacaagattttcaacagggcagaagaagaaagcaagaaagaaaaagacaatttctctaaccacacaacatgaggctacagacatgtcacaaagcagtggtgggagaaatgtagtctttggtgtaagaacttcagtttctgtaataattgccctccggcgagctgtgaaacgacaccgtgaacgacgagaaagacaaaagagagtcttcaggatgtctttattgattatttctacatttcttctctgctggacaccaatttctgttttaaataccaccattttatgtttaggcccaagtgaccttttagtaaaattaagattgtgttttttagtcatggcttatggaacaactatatttcaccctctattatatgcattcactagacaaaaatttcaaaaggtcttgaaaagtaaaatgaaaaagcgagttgtttctatagtagaagctgatcccctgcctaataatgctgtaatacacaactcttggatagatcctaaaagaaacaaaaaaattacctttgaagatagtgaaataagagaaaaacgtttagtgcctcaggttgtcacagactag

SEQ ID NO: 2 - protein, 433 aa, Homo sapiens
MCFSPILEINMQSESNITVRDDIDDINTNMYQPLSYPLSFQVSLTGFLMLEIVLGLGSNLTVLVLYCMKSNLINSVSNIITMNLHVLDVIICVGCIPLTIVILLLSLESNTALICCFHEACVSFASVSTAINVFAITLDRYDISVKPANRILTMGRAVMLMISIWIFSFFSFLIPFIEVNFFSLQSGNTWENKTLLCVSTNEYYTELGMYYHLLVQIPIFFFTVVVMLITYTKILQALNIRIGTRFSTGQKKKARKKKTISLTTQHEATDMSQSSGGRNVVFGVRTSVSVIIALRRAVKRHRERRERQKRVFRMSLLIISTFLLCWTPISVLNTTILCLGPSDLLVKLRLCFLVMAYGTTIFHPLLYAFTRQKFQKVLKSKMKKRVVSIVEADPLPNNAVIHNSWIDPKRNKKITFEDSEIREKRLVPQVVTD

SEQ ID NO: 3 - DNA, 1302 bp, Artificial Sequence
  substituted polynucleotide
atgtgcttctcccccatcctggagatcaacatgcagtcggagtcgaacatcacggtgcgggacgacatcgacgacatcaacacgaacatgtaccagccgctgtcctacccgctgagctttcaagtgtctctcaccgggttcctgatgctggagatcgtgctggggctgggcagcaacctcaccgtgctggtgctgtactgcatgaagtccaacctgatcaactcggtctccaacatcatcacgatgaacctgcacgtcctggacgtgatcatctgcgtggggtgcatccccctgacgatcgtgatcctgctgctgtccctggagagcaacacggcgctgatctgctgtttccatgaggcttgtgtgtcgttcgcgagcgtctcgaccgccatcaacgtgttcgccatcacgctggacagatacgacatctccgtcaagcccgccaaccggattctgaccatgggcagggcggtcatgctgatgatatccatttggatcttctcgttcttctcgttcctgatcccgttcatcgaggtgaacttcttcagcctgcagagcgggaacacctgggagaacaagaccctgctgtgcgtcagcaccaacgagtactacacggagctgggcatgtactaccacctgctggtccagatcccgatcttcttcttcacggtggtcgtcatgctgatcacctacaccaagatccttcaggcgctgaacatccggatcggcaccaggttctcgaccgggcagaagaagaaagcccggaagaagaagaccatctccctgaccacccagcacgaggctaccgacatgtcccagagcagcggcgggaggaacgtggtcttcggcgtccggacctccgtgtccgtgatcatcgccctccggcgagccgtgaagcggcaccgcgagcgccgggagcgacagaagagggtcttcaggatgagcctactgatcatctccacgttcctgctctgctggacgccgatctcggtcttgaacaccacgatcctgtgcctcggcccgagcgacctgctggtgaagctgcgactgtgcttcctggtcatggcctacgggacgacgatcttccacccgctgctgtacgcgttcacccgacagaagttccagaaggtcctgaagagtaagatgaagaagcgcgtcgtctccatcgtcgaagccgaccccctgccgaacaacgccgtcatccacaactcctggatcgaccccaagcgcaacaagaagatcaccttcgaggacagcgagatcagggagaagcgcctggtgcctcaggtcgtcaccgactag

SEQ ID NO: 4 - protein, 433 aa, Homo sapiens; identical to SEQ ID NO: 2

SEQ ID NO: 5 - DNA, 1302 bp, Homo sapiens
atgtgtttttctcccattctggaaatcaacatgcagtctgaatctaacattacagtgcgagatgacattgatgacatcaacaccaatatgtaccaaccactatcatatccgttaagctttcaagtgtctctcaccggatttcttatgttagaaattgtgttgggacttggcagcaacctcactgtattggtactttactgcatgaaatccaacttaatcaactctgtcagtaacattattacaatgaatcttcatgtacttgatgtaataatttgtgtgggatgtattcctctaactatagttatccttctgctttcactggagagtaacactgctctcatttgctgtttccatgaggcttgtgtatcttttgcaagtgtctcaacagcaatcaacgtttttgctatcactttggacagatatgacatctctgtaaaacctgcaaaccgaattctgacaatgggcagagctgtaatgttaatgatatccatttggattttttcttttttctctttcctgattccttttattgaggtaaattttttcagtcttcaaagtggaaatacctgggaaaacaagacacttttatgtgtcagtacaaatgaatactacactgaactgggaatgtattatcacctgttagtacagatcccaatattctttttcactgttgtagtaatgttaatcacatacaccaaaatacttcaggctcttaatattcgaataggcacaagattttcaacagggcagaagaagaaagcaagaaagaaaaagacaatttctctaaccacacaacatgaggctacagacatgtcacaaagcagtggtgggagaaatgtagtctttggtgtaagaacttcagtttctgtaataattgccctccggcgagctgtgaaacgacaccgtgaacgacgagaaagacaaaagagagtcttcaggatgtctttattgattatttctacatttcttctctgctggacaccaatttctgttttaaataccaccattttatgtttaggcccaagtgaccttttagtaaaattaagattgtgttttttagtcatggcttatggaacaactatatttcaccctctattatatgcattcactagacaaaaatttcaaaaggtcttgaaaagtaaaatgaaaaagcgagttgtttctatagtagaagctgatcccctgcctaataatgctgtaatacacaactcttggatagatcctaaaagaaacaaaaaaattacctttgaagatagtgaaataagagaaaaatgtttagtgcctcaggttgtcacagactag

SEQ ID NO: 6 - protein, 433 aa, Homo sapiens
MCFSPILEINMQSESNITVRDDIDDINTNMYQPLSYPLSFQVSLTGFLMLEIVLGLGSNLTVLVLYCMKSNLINSVSNIITMNLHVLDVIICVGCIPLTIVILLLSLESNTALICCFHEACVSFASVSTAINVFAITLDRYDISVKPANRILTMGRAVMLMISIWIFSFFSFLIPFIEVNFFSLQSGNTWENKTLLCVSTNEYYTELGMYYHLLVQIPIFFFTVVVMLITYTKILQALNIRIGTRFSTGQKKKARKKKTISLTTQHEATDMSQSSGGRNVVFGVRTSVSVIIALRRAVKRHRERRERQKRVFRMSLLIISTFLLCWTPISVLNTTILCLGPSDLLVKLRLCFLVMAYGTTIFHPLLYAFTRQKFQKVLKSKMKKRVVSIVEADPLPNNAVIHNSWIDPKRNKKITFEDSEIREKCLVPQVVTD

SEQ ID NO: 7 - DNA, 1302 bp, Artificial Sequence
  substituted polynucleotide
atgtgcttctcccccatcctggagatcaacatgcagtcggagtcgaacatcacggtgcgggacgacatcgacgacatcaacacgaacatgtaccagccgctgtcctacccgctgagctttcaagtgtctctcaccgggttcctgatgctggagatcgtgctggggctgggcagcaacctcaccgtgctggtgctgtactgcatgaagtccaacctgatcaactcggtctccaacatcatcacgatgaacctgcacgtcctggacgtgatcatctgcgtggggtgcatccccctgacgatcgtgatcctgctgctgtccctggagagcaacacggcgctgatctgctgtttccatgaggcttgtgtgtcgttcgcgagcgtctcgaccgccatcaacgtgttcgccatcacgctggacagatacgacatctccgtcaagcccgccaaccggattctgaccatgggcagggcggtcatgctgatgatatccatttggatcttctcgttcttctcgttcctgatcccgttcatcgaggtgaacttcttcagcctgcagagcgggaacacctgggagaacaagaccctgctgtgcgtcagcaccaacgagtactacacggagctgggcatgtactaccacctgctggtccagatcccgatcttcttcttcacggtggtcgtcatgctgatcacctacaccaagatccttcaggcgctgaacatccggatcggcaccaggttctcgaccgggcagaagaagaaagcccggaagaagaagaccatctccctgaccacccagcacgaggctaccgacatgtcccagagcagcggcgggaggaacgtggtcttcggcgtccggacctccgtgtccgtgatcatcgccctccggcgagccgtgaagcggcaccgcgagcgccgggagcgacagaagagggtcttcaggatgagcctactgatcatctccacgttcctgctctgctggacgccgatctcggtcttgaacaccacgatcctgtgcctcggcccgagcgacctgctggtgaagctgcgactgtgcttcctggtcatggcctacgggacgacgatcttccacccgctgctgtacgcgttcacccgacagaagttccagaaggtcctgaagagtaagatgaagaagcgcgtcgtctccatcgtcgaagccgaccccctgccgaacaacgccgtcatccacaactcctggatcgaccccaagcgcaacaagaagatcaccttcgaggacagcgagatcagggagaagtgcctggtgcctcaggtcgtcaccgactag

SEQ ID NO: 8 - protein, 433 aa, Homo sapiens; identical to SEQ ID NO: 6

SEQ ID NO: 9 - DNA, 1299 bp, Mus musculus
atgtgtttttctcctgttctggaaatcaacatgcagtctgaatcaaacgtcacggtgcgagatgacattgatgacatcgacaccaatatgtaccaaccactgtcatacccactaagctttcaagtgtctctcactggatttctcatgttagagatcgtgctggggcttggcagcaaccttaccgtcctggtactttactgcatgaaatccaacttaatcaactctgtcagtaacattattacaatgaacctccatgtacttgatgtcataatttgtgtgggatgcattcctctaactatagtgatccttctgctctcactggagagtaacactgctctcatctgctgtttccacgaagcttgtgtttcctttgcaagtgtttcgacagcaatcaacgtttttgctattactctggacagatatgacatctctgtaaaacctgcaaacagaattctgacaatgggcagagctgtaatgctaatgacatccatttggattttttctttcttctcattcctgattcccttcattgaagtaaattttttcagtcttcaaagtggaaatacatgggcaaacaagacactgctgtgtgtcagtacaagtgaatactatactgagctcgggatgtactatcaccttttggtgcagatccccatcttcttcttcacagttatagtcatgttgatcacatacactaagatactccaggctcttaacatccgcataggcactagattctcaacaggacagaagaagaaagcccgaaagaaaaagacaatctctctagctacacatgagaccacagacatgtcacaaagcagtggtgggaggaatgtcgtgtttggtgtgagaacttcagtttctgtaataattgccctccggcgagccgtgaaacgccaccgggaacgacgagaacggcagaaaagagtcttcaaaatgtcgttattgattatttctacatttcttctctgttggacaccaatttctgttttaaataccaccattctatgtttaggcccaagtgaccttttagtaaaattaagattgtgttttctagtcatggcttatggaacaacgatattccaccctctcttgtatgcattcaccagacaaaagtttcaaaaggtcttaaagagtaagatgaaaaagcgagttgtttccatagttgaagctgatcccatgcctaataacgctgtaatacacaactcatggatagatcctaaaagaaacaaaaaggttacctatgaagacagtgaaataagagagaaatgtttagtacctcaggttgtcacagactag

SEQ ID NO: 10 - protein, 432 aa, Mus musculus
MCFSPVLEINMQSESNVTVRDDIDDIDTNMYQPLSYPLSFQVSLTGFLMLEIVLGLGSNLTVLVLYCMKSNLINSVSNIITMNLHVLDVIICVGCIPLTIVILLLSLESNTALICCFHEACVSFASVSTAINVFAITLDRYDISVKPANRILTMGRAVMLMTSIWIFSFFSFLIPFIEVNFFSLQSGNTWANKTLLCVSTSEYYTELGMYYHLLVQIPIFFFTVIVMLITYTKILQALNIRIGTRFSTGQKKKARKKKTISLATHETTDMSQSSGGRNVVFGVRTSVSVIIALRRAVKRHRERRERQKRVFKMSLLIISTFLLCWTPISVLNTTILCLGPSDLLVKLRLCFLVMAYGTTIFHPLLYAFTRQKFQKVLKSKMKKRVVSIVEADPMPNNAVIHNSWIDPKRNKKVTYEDSEIREKCLVPQVVTD

SEQ ID NO: 11 - DNA, 1299 bp, Rattus norvegicus
atgtgtttttctcctgttctggaaatcaacatgcagtctgaatcaaacgtcacggtgcgagatgacattgaggacatcgataccaatatgtaccaaccactgtcatacccattaagctttcaagtgtctctcactggatttctcatgttagaaattgtgctggggcttggtagcaaccttaccgtactggtactttactgcatgaaatccaacttaatcagctctgtcagtaacattatcacaatgaatctccatgtacttgatgtaataatctgtgtgggatgtattcctctaaccatagtgatccttctgctctcactggagaggaacactgctctcatctgctgtttccacgaagcttgtgtttcttttgcaagtgtttccacagcaatcaacgtttttgctattactctggacagatatgacatctctgtaaaacctgcaaacagaattctgacaatgggcagagctgtgatgctaatgacgtccatttggattttttctttcttctcattcctgattcccttcattgaagtcaattttttcagccttcaaagtggaaatgcgtgggaaaacaagacactgctgtgtgtcagcacaagtgagtactacactgagctcgggatgtactatcacctcctagttcagatccccatcttcttcttcacagttatcgtgatgctgatcacatacaccaagatactccaggctcttaatatccggataggcactagattctcaacaggccagaagaagaaagcccggaagaaaaagacaatctctctaaccacacatgagaccacagacatgtcgcaaagcagtggtgggaggaatgtcgtatttggtgtgagaacttcagtttctgtaataattgccctccggcgagccgtgaaacgacaccgggaacgacgagagaggcagaaaagagtcttcaaaatgtcgttattgattatttctacatttcttctctgttggacaccaatttctgttttaaataccaccattttatgtttaggcccaagtgaccttttagtaaaattaagattgtgttttctagtcatggcttatggaacaactatattccatcctctcctgtatgcattcaccagacaaaaatttcaaaaggtcttaaaaagtaagatgaaaaagcgagttgtttccatagttgaagctgatcccatgcctaataacgctgtaatacacaactcatggatagatcctaaaagaaacaaaaaggttacctacgaagacagtgaaataagagagaaatgtttagtacctcaggttgtcacagactag

SEQ ID NO: 12 - protein, 432 aa, Rattus norvegicus
MCFSPVLEINMQSESNVTVRDDIEDIDTNMYQPLSYPLSFQVSLTGFLMLEIVLGLGSNLTVLVLYCMKSNLISSVSNIITMNLHVLDVIICVGCIPLTIVILLLSLERNTALICCFHEACVSFASVSTAINVFAITLDRYDISVKPANRILTMGRAVMLMTSIWIFSFFSFLIPFIEVNFFSLQSGNAWENKTLLCVSTSEYYTELGMYYHLLVQIPIFFFTVIVMLITYTKILQALNIRIGTRFSTGQKKKARKKKTISLTTHETTDMSQSSGGRNVVFGVRTSVSVIIALRRAVKRHRERRERQKRVFKMSLLIISTFLLCWTPISVLNTTILCLGPSDLLVKLRLCFLVMAYGTTIFHPLLYAFTRQKFQKVLKSKMKKRVVSIVEADPMPNNAVIHNSWIDPKRNKKVTYEDSEIREKCLVPQVVTD

SEQ ID NO: 13 - DNA, 1302 bp, Bos taurus
atgtgtttttctcccattctggaaatcaacatgcagtctgaatctaacattacagtgcgagatgacattgatgacatcaacaccaatatgtaccaaccactatcatatccattaagctttcaagtgtctctcaccggatttcttatgttagaaattgtgttgggacttggcagcaacctcaccgtattggtactttactgcatgaaatccaacttaatcaattctgtcagtaacattattacaatgaatcttcatgtacttgatgtaatcatttgtgtgggatgtattcctctaactatagttatccttctgctttcactggagagtaacactgctctcatctgctgtttccatgaggcctgtgtatcttttgcaagtgtctcaacagcaatcaacgtgttcgctatcactttggacagatacgacatctctgtaaagcctgcaaaccgaattctgacaatgggcagagctgcaatgctaatgatatccatttggattttttcgtttttctctttcctgattccctttattgaggtaaattttttcagccttcaaagtggaaatacatgggaaaacaagacacttttgtgtgtcagcacaaatgaatactacactgaactgggaatgtattatcacctgctagtacagattccaatattctttttcactgtcatagtgatgttaatcacatacagcaaaatacttcaggctctcaatattcgaataggcacaagattctcaacagggcagaagaagaaagcaagaaagaaaaagacaatctctctaaccacacaacacgagactacagacatgtcacaaggcagtggtgggagaaatgtagtctttggtgttagaacttcagtgtctgtaataatcgctctccggcgagctatgaaacgacaccgtgaacgacgagaaaggcaaaagagagtcttcaaaatgtctttattgattatttctacatttcttctctgctggacaccaatttctgttttaaataccaccattttatgtttaggcccaagtgaccttttagtaaaattaaggttgtgttttctagtcatgggttatggaacaactatatttcaccctctattatatgcatttactagacaaaaatttcaaaaggtcttaaaaagtaaaatgaaaaagcgagttgtttccatagtagaagctgatcccatgcctaataatgctgtaatacacaactcttggatagatcctaaaagaaacaaaaaactcacctttgaagatagtgaaataagagaaaaatgtttagtacctcaggttgtcacagactag

SEQ ID NO: 14 - protein, 433 aa, Bos taurus
MCFSPILEINMQSESNITVRDDIDDINTNMYQPLSYPLSFQVSLTGFLMLEIVLGLGSNLTVLVLYCMKSNLINSVSNIITMNLHVLDVIICVGCIPLTIVILLLSLESNTALICCFHEACVSFASVSTAINVFAITLDRYDISVKPANRILTMGRAAMLMISIWIFSFFSFLIPFIEVNFFSLQSGNTWENKTLLCVSTNEYYTELGMYYHLLVQIPIFFFTVIVMLITYSKILQALNIRIGTRFSTGQKKKARKKKTISLTTQHETTDMSQGSGGRNVVFGVRTSVSVIIALRRAMKRHRERRERQKRVFKMSLLIISTFLLCWTPISVLNTTILCLGPSDLLVKLRLCFLVMGYGTTIFHPLLYAFTRQKFQKVLKSKMKKRVVSIVEADPMPNNAVIHNSWIDPKRNKKLTFEDSEIREKCLVPQVVTD

SEQ ID NO: 15 - protein, 6 aa, Artificial Sequence
  Synthetic Oligopeptide
TLESIM

SEQ ID NO: 16 - protein, 5 aa, Artificial Sequence
  Synthetic Oligopeptide
EYNLV

SEQ ID NO: 17 - protein, 5 aa, Artificial Sequence
  Synthetic Oligopeptide
DCGLF

SEQ ID NO: 18 - DNA, 36 bp, Artificial Sequence
  primer
gatcaagcttccatggcgtgctgcctgagcgaggag

SEQ ID NO: 19 - DNA, 53 bp, Artificial Sequence
  primer
gatcggatccttagaacaggccgcagtccttcaggttcagctgcaggatggtg

SEQ ID NO: 20 - DNA, 1062 bp, Artificial Sequence
  chimeric polynuceotide
atggcgtgctgcctgagcgaggaggccaaggaagcccggaggatcaacgacgagatcgagcggcagctgcgcagggacaagcgcgacgcccgccgggagctcaagctgctgctgctggggacaggggagagtggcaagtcgaccttcatcaagcagatgaggatcatccacgggtcgggctactctgacgaagacaagcgcggcttcaccaagctggtgtatcagaacatcttcacggccatgcaggccatgatcagagcgatggacacactcaagatcccatacaagtatgaacacaataaggctcatgcacaattggttcgagaggttgatgtggagaaggtgtctgcttttgacgtccccgactacgcggcaataaagagcttgtggaatgatcctggaatccaggagtgctacgacagacgacgggaatatcagttatctgactctaccaaatactatctgaatgacttggaccgtgtagccgacccttcctatctgcctacacaacaagacgtgcttagagttcgagtccccactacagggatcatcgaatacccctttgacttacaaagtgtcattttcagaatggtcgatgtagggggccaaaggtcagagagaagaaaatggatccactgctttgaaaatgtcacctccatcatgtttctagtagcgcttagcgaatatgatcaagttcttgtggagtcagacaatgagaaccgcatggaggagagcaaagcactctttagaacaattatcacctacccctggttccagaactcctctgtgattctgttcttaaacaagaaagatcttctagaggagaaaatcatgtattcccacctagtcgactacttcccagaatatgatggaccccagagagatgcccaggcagctcgagaattcatcctgaaaatgttcgtggacctgaaccccgacagtgacaaaatcatctactcccacttcacgtgcgccacagataccgagaacatccgcttcgtctttgcagccgtcaaggacaccatcctgcagctgaacctgaaggactgcggcctgttctaa

SEQ ID NO: 21 - protein, 353 aa, Artificial Sequence
  chimeric polypeptide
MACCLSEEAKEARRINDEIERQLRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFDVPDYAAIKSLWNDPGIQECYDRRREYQLSDSTKYYLNDLDRVADPSYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLKDCGLF

SEQ ID NO: 22 - DNA, 545 bp, Artificial Sequence
  fragment of substituted polynucleotide
atcaacgtgttcgccatcacgctggacagatacgacatctccgtcaagcccgccaaccggattctgaccatgggcagggcggtcatgctgatgatatccatttggatcttctcgttcttctcgttcctgatcccgttcatcgaggtgaacttcttcagcctgcagagcgggaacacctgggagaacaagaccctgctgtgcgtcagcaccaacgagtactacacggagctgggcatgtactaccacctgctggtccagatcccgatcttcttcttcacggtggtcgtcatgctgatcacctacaccaagatccttcaggcgctgaacatccggatcggcaccaggttctcgaccgggcagaagaagaaagcccggaagaagaagaccatctccctgaccacccagcacgaggctaccgacatgtcccagagcagcggcgggaggaacgtggtcttcggcgtccggacctccgtgtccgtgatcatcgccctccggcgagccgtgaagcggcaccgcgagcgccgggagcgacagaagagggtctt

(19) United States
(12) Patent Application Publication
(10) Pub. No.: US 2009/0313708 A1
(43) Pub. Date: Dec. 17, 2009

(54) GPR22 AND METHODS RELATING THERETO

(76) Inventor: Chen W. Liaw, San Diego, CA (US)

Correspondence Address: Arena Pharmaceuticals, Inc.;Bozicevic, Field & Francis LLP, 1900 University Avenue, Suite 200, East Palo Alto, CA, 94303, US

(21) Appl. No.: 12/089,893

(22) PCT Filed: Oct. 12, 2006
(86) PCT No.: PCT/US2006/040226

§ 371 Date: Sep. 5, 2008

Related U.S. Application Data
(60) Provisional application No. 60/727,203, filed on Oct. 14, 2005.

(51) Int. Cl. G01N 33/00; C12Q 1/68; C07H 21/04; C12N 15/63; C12N 5/00; C12P 21/06; C12Q 1/02; A01K 67/00

(52) U.S. Cl. 800/3; 435/6; 536/23.1; 435/320.1; 435/325; 435/69.1; 435/29; 800/13

(57) ABSTRACT

Methods for generating an expression-enhanced GPR22 nucleic acid, as well as substituted GPR22 nucleic acids providing for enhanced expression of the encoded GPR22 polypeptide, are provided. In practicing the subject methods, a nucleic acid encoding a mammalian GPR22 receptor polypeptide (e.g., a wild-type nucleic acid) is expression-enhanced by identifying the various codons of the coding region for the GPR22 amino acid sequence and substituting nucleotides so as to enhance expression without changing the amino acid sequence of the encoded GPR22 polypeptide. Methods, compositions, and kits using the same for screening of modulators of GPR22 are also provided.

### BACKGROUND OF THE INVENTION

Congestive heart failure (CHF) affects nearly 5 million Americans and there are over 500,000 new cases diagnosed annually. CHF is a clinical syndrome that reduces cardiac output, increases venous pressures, and is accompanied by molecular abnormalities that cause progressive deterioration of the failing heart and premature myocyte death.

There are currently few to no drugs clinically available that are designed to inhibit cardiac myocyte death or to directly activate survival pathways. Such drugs would be useful for improving cardiac function and promoting survival. Accordingly, the development of therapeutic strategies for the treatment and prophylaxis of human heart failure hold great promise.

GPR22 is a G protein-coupled receptor (GPCR) expressed by cardiac myocytes (cardiomyocytes) and shown to play a role in cardioprotection (to confer cardioprotection). The down regulation of GPR22 in cardiomyocytes has been linked to ischemia and other heart diseases associated with cardiomyocyte apoptosis, such as CHF. GPR22 is encoded by a single exon and exhibits detectable constitutive activity consistent with GPR22 being coupled to Gi.

Identification of modulators of GPR22 is of interest, as such agents can have therapeutic and prophylactic effects on cardiac output, venous pressure, myocardial infarction, CHF, ischemic heart disease, myocyte apoptosis, and the like. Assays to identify such GPR22 modulators are generally conducted in a cell-based system. It is useful, therefore, to express the GPR22 polypeptide at sufficiently high levels so as to facilitate its use in cell based assay systems wherein modulators of the GPR22 polypeptide are to be screened. Moreover, providing for increased cell surface expression of GPR22 would enhance the efficiency of such assays.

The present invention meets that objective.

Literature

WO 2004/013285; U.S. Pat. No. 5,994,097; O'Dowd et al., Gene (1997) 187:75-81; Guhaniyogi et al., Gene (2001) 265:11-23; Cello et al., Science (2002) 297:1016-1018; Bemal, Gene (2005) PMID: 15922516; Fujimori et al., BMC Genomics (2005) 6(1):26; Semon et al., Hum Mol. Genet. (2005) 14(3): 421-427.

### SUMMARY OF THE INVENTION

Methods for generating an expression-enhanced GPR22 nucleic acid, as well as substituted GPR22 nucleic acids providing for enhanced expression of the encoded GPR22 polypeptide, are provided. In practicing the subject methods, a nucleic acid encoding a mammalian GPR22 receptor polypeptide (e.g., a wild-type nucleic acid) is expression-enhanced by identifying the various codons of the coding region for the GPR22 amino acid sequence and substituting nucleotides so as to enhance expression without changing the amino acid sequence of the encoded GPR22 polypeptide. Methods, compositions, and kits using the same for screening of modulators of GPR22 are also provided.

In a first aspect, the invention features a method for modifying a first nucleic acid encoding a mammalian GPR22 receptor amino acid sequence to provide for enhanced expression of the encoded mammalian GPR22 receptor polypeptide in a eukaryotic host cell, comprising the steps of:
(a) identifying a codon in the mammalian GPR22 receptor coding region for said first nucleic acid that comprises a target nucleotide, said target nucleotide being an adenine that is capable of being substituted with a guanine, a cytosine or a thymine without changing the amino acid encoded by the codon, or said target nucleotide being a thymine that is capable of being substituted with a guanine, a cytosine or an adenine without changing the amino acid encoded by the codon; and
(b) substituting said target nucleotide which is an adenine with a guanine, a cytosine or a thymine or said target nucleotide which is a thymine with a guanine, a cytosine or an adenine to generate a non-endogenous substituted nucleic acid encoding the mammalian GPR22 receptor amino acid sequence;
wherein the generating of said non-endogenous substituted nucleic acid provides for enhanced expression of the encoded mammalian GPR22 receptor polypeptide in the eukaryotic host cell, wherein said enhanced expression is in comparison to the first nucleic acid or to a wild-type nucleic acid encoding the mammalian GPR22 receptor polypeptide.

In some embodiments, the target nucleotide which is an adenine is substituted with a guanine or a cytosine.

In some embodiments, the target nucleotide which is a thymine is substituted with a guanine or a cytosine.

In some embodiments, the target nucleotide which is an adenine or which is a thymine is substituted with a guanine or a cytosine.

In some embodiments, the mammalian GPR22 receptor amino acid sequence is a wild-type mammalian GPR22 receptor amino acid sequence. In some embodiments, the wild-type mammalian GPR22 receptor amino acid sequence is a wild-type human GPR22 receptor amino acid sequence. In some embodiments, the wild-type mammalian GPR22 receptor amino acid sequence is a wild-type human GPR22R425 or GPR22 C425 amino acid sequence. In some embodiments, the wild-type mammalian GPR22 receptor amino acid sequence is SEQ ID NO: 2 or SEQ ID NO: 6.

In some embodiments, the first nucleic acid is a wild-type mammalian GPR22 receptor nucleic acid. In some embodiments, the first nucleic acid is a wild-type human GPR22 receptor nucleic acid. In some embodiments, the first nucleic acid is a wild-type human GPR22R425 or C425 nucleic acid. In some embodiments, the first nucleic acid is SEQ ID NO: 1 or SEQ ID NO: 5.

In some embodiments, the non-endogenous substituted nucleic acid is SEQ ID NO: 3 or SEQ ID NO: 7.

In some embodiments, the wild-type nucleic acid encoding the mammalian GPR22 receptor polypeptide is a wild-type human GPR22 nucleic acid. In some embodiments, the wild-type nucleic acid encoding the mammalian GPR22 receptor polypeptide is a wild-type human GPR22R425 or GPR22C425 nucleic acid. In some embodiments, the wild-type nucleic acid encoding the mammalian GPR22 receptor polypeptide is SEQ ID NO: 1 or SEQ ID NO: 5.

In some embodiments, the eukaryotic host cell is a mammalian cell.

In some embodiments, the eukaryotic host cell is a melanophore cell.

In some embodiments, the eukaryotic host cell is a yeast cell.

In some embodiments, step (b) is repeated for every target nucleotide that comprises the identified codon.

In some embodiments, steps (a) to (b) are repeated for up to every codon in said coding region of the mammalian GPR22 receptor amino acid sequence that comprises a target nucleotide.

In some embodiments, steps (a) to (b) are repeated so that at least about 10%, at least about 15%, at least about 20%, at least about 25%, at least about 30%, at least about 35%, at least about 40%, at least about 45%, at least about 50%, at least about 55%, or at least about 60% or more of all codons in said coding region of the mammalian GPR22 receptor amino acid sequence have at least one target nucleotide substituted. In some embodiments, steps (a) to (b) are repeated so that at least about 60% of all codons in said coding region of the mammalian GPR22 receptor amino acid sequence have at least one target nucleotide substituted.

In some embodiments, steps (a) to (b) are repeated so that an adenine or a thymine in at least about 10%, at least about 20%, at least about 30%, at least about 40%, at least about 50%, at least about 60%, at least about 70%, at least about 75%, at least about 80%, at least about 85%, at least about 90%, or at least about 95% of the codons in said coding region comprising a target nucleotide is substituted with a guanine or a cytosine. In some embodiments, steps (a) to (b) are repeated so that an adenine or a thymine in at least about 75%, at least about 80%, at least about 85%, or at least about 90% of the codons in said coding region comprising a target nucleotide is substituted with a guanine or a cytosine. In some embodiments, steps (a) to (b) are repeated so that an adenine or a thymine in 100% of the codons in said coding region comprising a target nucleotide is substituted with a guanine or a cytosine.

In some embodiments, steps (a) to (b) are repeated so that the GC-content of the coding region of the substituted non-endogenous nucleic acid encoding the mammalian GPR22 receptor amino acid sequence is increased by at least about 10%, by at least about 15%, by at least about 20%, by at least about 25%, by at least about 30%, by at least about 35%, by at least about 40%, by at least about 45%, by at least about 50%, by at least about 55%, by at least about 60%, or by at least about 65%, or more in comparison with the coding region of the first nucleic acid encoding the mammalian GPR22 amino acid sequence. In some embodiments, steps (a) to (b) are repeated so that the GC-content of the coding region of the substituted non-endogenous nucleic acid encoding the mammalian GPR22 receptor amino acid sequence is increased by at least about 50%, at least about 55%, or at least about 60% in comparison with the coding region of the first nucleic acid encoding the mammalian GPR22 amino acid sequence.

In some embodiments, steps (a) to (b) are repeated so that the GC-content of the coding region of the substituted nucleic acid encoding the mammalian GPR22 receptor amino acid sequence is at least about 35%, at least about 36%, at least about 37%, at least about 38%, at least about 39%, at least about 40%, at least about 45%, at least about 50%, at least about 55%, or at least about 60%. In some embodiments, steps (a) to (b) are repeated so that the GC-content of the coding region of the substituted nucleic acid encoding the mammalian GPR22 receptor amino acid sequence is at least about 45%, at least about 50%, or at least about 55%.

In some embodiments, said target nucleotide is one of three or more contiguous adenines or thymines within said coding region.

In a second aspect, the invention features a method for modifying a first nucleic acid encoding a mammalian GPR22 receptor amino acid sequence to provide for enhanced expression of the encoded mammalian GPR22 receptor polypeptide in a eukaryotic host cell, comprising the steps of a method according to the first aspect, and further comprising:
(c) comparing in the eukaryotic host cell a first level of expression of the mammalian GPR22 receptor polypeptide encoded by the non-endogenous substituted nucleic acid with a second level of expression of the mammalian GPR22 receptor polypeptide encoded by the first nucleic acid or by the wild-type nucleic acid encoding the mammalian GPR22 receptor polypeptide;
wherein said first level of expression of the mammalian GPR22 receptor polypeptide greater than said second level of expression of the mammalian GPR22 receptor polypeptide for the first nucleic acid or said second level of expression of the mammalian receptor polypeptide for the wild-type nucleic acid is indicative of the non-endogenous substituted nucleic acid providing for enhanced expression of the encoded mammalian GPR22 receptor polypeptide in the eukaryotic host cell.

In some embodiments, said comparing is by a process comprising measuring a level of receptor functionality. In some embodiments, said process comprises measuring a level of a second messenger. In some embodiments, said process comprises measuring a level of a second messenger selected from the group consisting of cyclic AMP (cAMP), cyclic GMP (cGMP), inositol triphosphate (IP₃), diacylglycerol (DAG), Ca²⁺ and MAP kinase activity. In some embodiments, said process comprises measuring a level of intracellular IP₃ accumulation. In some embodiments, said process comprises measuring a level of intracellular Ca²⁺. In some embodiments, said process comprises measuring a level of intracellular cAMP.

In certain embodiments, said process comprises measuring an increase in or stimulation of intracellular IP₃ accumulation. In certain embodiments, the increase in or stimulation of intracellular IP₃ accumulation for the non-endogenous substituted nucleic acid is at least about 130%, at least about 150%, at least about 200%, at least about 250%, at least about 300%, at least about 350%, at least about 400%, at least about 450%, at least about 500%, at least about 550%, at least about 600%, at least about 650%, at least about 700%, at least about 750%, at least about 800%, at least about 850%, at least about 900%, at least about 950%, or at least about 1000% the increase in or stimulation of intracellular IP₃ accumulation for the wild-type nucleic acid. In certain embodiments, the increase in or stimulation of intracellular IP₃ accumulation for the non-endogenous substituted nucleic acid is at least about 200%, at least about 300%, at least about 400%, at least about 500%, at least about 600%, at least about 700%, at least about 800%, at least about 900%, or at least about 1000% the increase in or stimulation of intracellular IP₃ accumulation for the wild-type nucleic acid. In certain embodiments, the increase in or stimulation of intracellular IP₃ accumulation for the non-endogenous substituted nucleic acid is at least about 400%, at least about 500%, at least about 600%, at least about 700%, or at least about 800% the increase in or stimulation of intracellular IP₃ accumulation for the wild-type nucleic acid. In certain embodiments, an increase in or stimulation of intracellular IP₃ accumulation for the non-endogenous substituted nucleic acid of at least about 130%, at least about 150%, at least about 200%, at least about 250%, at least about 300%, at least about 350%, at least about 400%, at least about 450%, at least about 500%, at least about 550%, at least about 600%, at least about 650%, at least about 700%, at least about 750%, at least about 800%, at least about 850%, at least about 900%, at least about 950%, or at least about 1000% the increase in or stimulation of intracellular IP₃ accumulation for the wild-type nucleic acid is indicative of the non-endogenous substituted nucleic acid providing for enhanced expression of the encoded mammalian GPR22 receptor. In certain embodiments, an increase in or stimulation of intracellular IP₃ accumulation for the non-endogenous substituted nucleic acid of at least about 200%, at least about 300%, at least about 400%, at least about 500%, at least about 600%, at least about 700%, at least about 800%, at least about 900%, or at least about 1000% the increase in or stimulation of intracellular IP₃ accumulation for the wild-type nucleic acid is indicative of the non-endogenous substituted nucleic acid providing for enhanced expression of the encoded mammalian GPR22 receptor. In certain embodiments, an increase in or stimulation of intracellular IP₃ accumulation for the non-endogenous substituted nucleic acid of at least about 400%, at least about 500%, at least about 600%, at least about 700%, or at least about 800% the increase in or stimulation of intracellular IP₃ accumulation for the wild-type nucleic acid is indicative of the non-endogenous substituted nucleic acid providing for enhanced expression of the encoded mammalian GPR22 receptor. In certain embodiments, said process comprises measuring intracellular IP₃ accumulation in a cell comprising Gq(del)/Gi chimeric G protein.

In certain embodiments, said process comprises measuring a decrease in or suppression of intracellular cAMP accumulation. In certain embodiments, the decrease in or suppression of intracellular cAMP accumulation for the non-endogenous substituted nucleic acid is at least about 1.5 times, at least about 2.0 times, at least about 2.5 times, at least about 3.0 times, at least about 3.5 times, at least about 4.0 times, at least about 4.5 times, or at least about 5.0 times the decrease in or suppression of intracellular cAMP accumulation for the wild-type nucleic acid. In certain embodiments, the decrease in or suppression of intracellular cAMP accumulation for the non-endogenous substituted nucleic acid is at least about 2.0 times, at least about 2.5 times, or at least about 3.0 times the decrease in or suppression of intracellular cAMP accumulation for the wild-type nucleic acid. In certain embodiments, a decrease in or suppression of intracellular cAMP accumulation for the non-endogenous substituted nucleic acid of at least about 1.5 times, at least about 2.0 times, at least about 2.5 times, at least about 3.0 times, at least about 3.5 times, at least about 4.0 times, at least about 4.5 times, or at least about 5.0 times the decrease in or suppression of intracellular cAMP accumulation for the wild-type nucleic acid is indicative of the non-endogenous substituted nucleic acid providing for enhanced expression of the encoded mammalian GPR22 receptor. In certain embodiments, a decrease in or suppression of intracellular cAMP accumulation for the non-endogenous substituted nucleic acid of at least about 1.5 times, at least about 2.0 times, at least about 2.5 times, or at least about 3.0 times the decrease in or suppression of intracellular cAMP accumulation for the wild-type nucleic acid is indicative of the non-endogenous substituted nucleic acid providing for enhanced expression of the encoded mammalian GPR22 receptor. In certain embodiments, said process comprises measuring intracellular cAMP accumulation in a cell comprising a signal enhancer.

In some embodiments, said comparing is by a process comprising measuring a level of steady-state GPR22 receptor polypeptide expression.

In some embodiments, said comparing is by a process comprising measuring a level of steady-state GPR22 receptor mRNA expression.

In a third aspect, the invention features an isolated polynucleotide comprising a non-endogenous substituted nucleic acid encoding a mammalian GPR22 receptor according to, as set forth in, or generated according to a method of the first or second aspect. In some embodiments, the isolated polynucleotide comprises a non-endogenous substituted nucleic acid encoding a mammalian GPR22 receptor, wherein the non-endogenous substituted nucleic acid is according to, set forth in, or generated according to a method of the first or second aspect. In certain embodiments, the isolated polynucleotide comprises a non-endogenous substituted nucleic acid encoding a mammalian GPR22 receptor, wherein the non-endogenous substituted nucleic acid is SEQ ID NO:3 or SEQ ID NO:7.

In a fourth aspect, the invention features a vector comprising the isolated polynucleotide of the third aspect. In some embodiments, the vector is an expression vector and the polynucleotide is operably linked to a promoter.

In certain embodiments, the isolated polynucleotide comprises a non-endogenous substituted nucleic acid encoding a mammalian GPR22 receptor, wherein the non-endogenous substituted nucleic acid is SEQ ID NO:3 or SEQ ID NO:7.

In a fifth aspect, the invention features a recombinant host cell comprising a vector according to the fourth aspect. In some embodiments, the vector is an expression vector and the polynucleotide is operably linked to a promoter.

In certain embodiments, the isolated polynucleotide comprises a non-endogenous substituted nucleic acid encoding a mammalian GPR22 receptor, wherein the non-endogenous substituted nucleic acid is SEQ ID NO:3 or SEQ ID NO:7.

In a sixth aspect, the invention features a method for producing a recombinant host cell comprising:
(a) transfecting an expression vector according to the fourth aspect into a eukaryotic host cell to thereby produce a transfected host cell; and
(b) culturing the transfected host cell under conditions sufficient to express the mammalian GPR22 receptor from the expression vector.

In some embodiments, the host cell is a mammalian cell.

In some embodiments, the host cell is a melanophore cell.

In some embodiments, the host cell is a yeast cell.

In certain embodiments, the isolated polynucleotide comprises a non-endogenous substituted nucleic acid encoding a mammalian GPR22 receptor, wherein the non-endogenous substituted nucleic acid is SEQ ID NO:3 or SEQ ID NO:7.

In a seventh aspect, the invention features a method for identifying a candidate compound as a modulator of a mammalian GPR22 receptor, said method comprising the steps of:
(a) contacting the candidate compound with the mammalian GPR22 receptor that comprises a recombinant host cell according to the sixth aspect or membrane of the host cell or with a recombinant host cell produced according to a method of the sixth aspect or membrane thereof comprising the mammalian GPR22 receptor, wherein the mammalian GPR22 receptor couples to a G protein; and
(b) determining the ability of the candidate compound to inhibit or stimulate functionality of the mammalian GPR22 receptor;
   wherein the ability of the candidate compound to inhibit or stimulate said functionality is indicative of the candidate compound being a modulator of the mammalian GPR22 receptor.

In certain embodiments, the method further comprises producing a recombinant host cell by a method according to the sixth aspect.

In certain embodiments, the method further comprises providing a recombinant host cell produced by a method of the sixth aspect.

In some embodiments, the G protein is Gi.

In some embodiments, the G protein is Gq(del)/Gi chimeric G protein.

In some embodiments, said determining is by a process comprising measuring a level of a second messenger. In some embodiments, said determining is by a process comprising measuring a level of a second messenger selected from the group consisting of cyclic AMP (cAMP), cyclic GMP (cGMP), inositol triphosphate (IP₃), diacylglycerol (DAG), Ca²⁺ and MAP kinase activity. In some embodiments, said process comprises measuring a level of intracellular IP₃ accumulation. In some embodiments, said process comprises measuring a level of intracellular Ca²⁺. In some embodiments, said process comprises measuring a level of intracellular cAMP.

In some embodiments, the candidate compound is a small molecule. In some embodiments, the candidate compound is a small molecule, with the proviso that the small molecule is not a polypeptide. In some embodiments, the candidate compound is a small molecule, with the proviso that the small molecule is not an antibody or an antigen-binding fragment thereof. In some embodiments, the candidate compound is a small molecule, with the proviso that the small molecule is not a lipid. In some embodiments, the candidate compound is a small molecule, with the proviso that the small molecule is not a polypeptide or a lipid. In some embodiments, the candidate compound is a polypeptide. In some embodiments, the candidate compound is a polypeptide, with the proviso that the polypeptide is not an antibody or an antigen-binding fragment thereof. In some embodiments, the candidate compound is a lipid. In some embodiments, the candidate compound is not an antibody or an antigen-binding fragment thereof. In some embodiments, the candidate compound is an antibody or an antigen-binding fragment thereof. In some embodiments, the candidate compound is not an endogenous ligand of the mammalian GPR22 receptor.

In some embodiments, the candidate compound is a small molecule. In some embodiments, the candidate compound is not an antibody or an antigen-binding fragment thereof.

In some embodiments, the modulator is selected from the group consisting of agonist, partial agonist, inverse agonist, and antagonist. In certain embodiments, the method comprises identifying an agonist, partial agonist, inverse agonist or antagonist of the mammalian GPR22 receptor. In certain embodiments, the method further comprises the step of formulating said agonist, partial agonist, inverse agonist or antagonist as a pharmaceutical. In certain embodiments, the method comprises identifying an agonist or partial agonist of the mammalian GPR22 receptor.

In certain embodiments, the isolated polynucleotide comprises a non-endogenous substituted nucleic acid encoding a mammalian GPR22 receptor, wherein the non-endogenous substituted nucleic acid is SEQ ID NO:3 or SEQ ID NO:7.

In an eighth aspect, the invention features a method for identifying a candidate compound as a ligand of a mammalian GPR22 receptor, said method comprising the steps of:
(a) contacting the candidate compound with the mammalian GPR22 receptor that comprises a recombinant host cell according to the sixth aspect or membrane of the host cell or with a recombinant host cell produced according to a method of the sixth aspect or membrane thereof comprising the mammalian GPR22 receptor; and
(b) measuring the ability of the compound to bind to the mammalian GPR22 receptor;
wherein said binding is indicative of the candidate compound being a ligand of the mammalian GPR22 receptor.

In certain embodiments, the method further comprises producing a recombinant host cell by a method according to the sixth aspect.

In certain embodiments, the method further comprises providing a recombinant host cell produced by a method of the sixth aspect.

The invention also features a method for identifying a candidate compound as a ligand of a mammalian GPR22 receptor, said method comprising the steps of:
(a) contacting an optionally labeled known ligand of the mammalian GPR22 receptor with the mammalian GPR22 receptor that comprises a recombinant host cell according to the sixth aspect or membrane of the host cell or with a recombinant host cell produced according to a method of the sixth aspect or membrane thereof comprising the mammalian GPR22 receptor in the presence or absence of the candidate compound;
(b) detecting the complex between said known ligand and the mammalian GPR22 receptor; and
(c) determining whether less of said complex is formed in the presence of the candidate compound than in the absence of the candidate compound;
wherein said determination is indicative of the candidate compound being a ligand of the mammalian GPR22 receptor.

In certain embodiments, the method further comprises producing a recombinant host cell by a method according to the sixth aspect.

In certain embodiments, the method further comprises providing a recombinant host cell produced by a method of the sixth aspect.

In some embodiments, the optionally labeled known ligand is radiolabeled.

In some embodiments, the candidate compound is a small molecule. In some embodiments, the candidate compound is a small molecule, with the proviso that the small molecule is not a polypeptide. In some embodiments, the candidate compound is a small molecule, with the proviso that the small molecule is not an antibody or an antigen-binding fragment thereof. In some embodiments, the candidate compound is a small molecule, with the proviso that the small molecule is not a lipid. In some embodiments, the candidate compound is a small molecule, with the proviso that the small molecule is not a polypeptide or a lipid. In some embodiments, the candidate compound is a polypeptide. In some embodiments, the candidate compound is a polypeptide, with the proviso that the polypeptide is not an antibody or an antigen-binding fragment thereof. In some embodiments, the candidate compound is a lipid. In some embodiments, the candidate compound is not an antibody or an antigen-binding fragment thereof. In some embodiments, the candidate compound is an antibody or an antigen-binding fragment thereof. In some embodiments, the candidate compound is not an endogenous ligand of the mammalian GPR22 receptor.

In some embodiments, the candidate compound is a small molecule. In some embodiments, the candidate compound is not an antibody or an antigen-binding fragment thereof.

In certain embodiments, the method further comprises the step of formulating said ligand as a pharmaceutical.

In certain embodiments, the isolated polynucleotide comprises a non-endogenous substituted nucleic acid encoding a mammalian GPR22 receptor, wherein the non-endogenous substituted nucleic acid is SEQ ID NO:3 or SEQ ID NO:7.

In a ninth aspect, the invention features a method according to the seventh or eighth aspect further comprising the step of formulating the modulator or ligand into a pharmaceutical composition. The invention also features a method according to the seventh or eighth aspect further comprising the step of resynthesizing the modulator or ligand.

In a tenth aspect, the invention features a non-human mammal transgenic for a human GPR22 receptor, wherein the human GPR22 receptor is encoded by or expressed from a polynucleotide according to the third aspect.

In some embodiments, the non-human mammal is a mouse, a rat, or a pig.

Methods for making a transgenic non-human mammal are well known in the art. See, e.g., Wall et al, J Cell Biochem (1992) 49:113-120; Hogan et al, in Manipulating the Mouse Embryo. A Laboratory Manual. (1986) Cold Spring Harbor Laboratory Press, Cold Spring Harbor, N.Y.; Costa et al, FASEB J (1999) 13:1762-1773; WO 91/08216; U.S. Pat. No. 4,736,866; and U.S. Pat. No. 6,504,080; the disclosure of each of which is herein incorporated by reference in its entirety.

In some embodiments, the expression of the human GPR22 receptor is cardiomyocyte selective. In some embodiments, said cardiomyocyte-selective expression of the human GPR22 receptor is conferred by alpha myosin heavy chain promoter [Subramaniam et al, J Biol Chem (1991) 266:24613-24620; the disclosure of which is herein incorporated by reference in its entirety].

In some embodiments, the expression of the human GPR22 is neuron selective.

In certain embodiments, the polynucleotide according to the third aspect comprises a non-endogenous substituted nucleic acid encoding a mammalian GPR22 receptor, wherein the non-endogenous substituted nucleic acid is SEQ ID NO:3 or SEQ ID NO:7.

In an eleventh aspect, the invention features a method of using a transgenic non-human mammal according to the tenth aspect to identify whether a candidate compound has efficacy for preventing or treating a disease or disorder related to a mammalian GPR22 receptor comprising the step of administering the candidate compound to the transgenic non-human mammal. In certain embodiments, said efficacy in the transgenic non-human mammal is indicative of efficacy in a mammal.

In certain embodiments, the candidate compound is a modulator or ligand of the human GPR22 receptor. In some embodiments, the candidate compound is not an endogenous ligand of the human GPR22 receptor.

In some embodiments, the disease or disorder related to the mammalian GPR22 receptor is myocardial ischemia or a condition related thereto, including but not limited to myocardial infarction. In some embodiments, the disease or disorder related to the mammalian GPR22 receptor is congestive heart failure. In some embodiments, the disease or disorder related to the mammalian GPR22 receptor is cerebral ischemia or a condition related thereto, including but not limited to ischemic stroke.

Routes of administering the candidate compound to a non-human mammal are well known in the art and include but are not limited to oral, intraperitoneal, subcutaneous and intravenous administration.

In some embodiments, the dose of the compound is 0.1-100 mg/kg. In some embodiments, the dose is selected from the group consisting of 0.1 mg/kg, 0.3 mg/kg, 1.0 mg/kg, 3.0 mg/kg, 10 mg/kg, 30 mg/kg and 100 mg/kg.

Methods for identifying whether a candidate compound has efficacy for myocardial infarction in a mammal are well known in the art (see, e.g., Fryer et al, Circ Res (1999) 84:846-851; the disclosure of which is herein incorporated by reference in its entirety). Methods for identifying whether a candidate compound has efficacy for congestive heart failure in a mammal are well known in the art (see, e.g., Wang et al, J Pharmacol Toxicol Methods (2004) 50:163-174; the disclosure of which is herein incorporated by reference in its entirety). Methods for identifying whether a candidate compound has efficacy for ischemic stroke in a mammal are well known in the art (see, e.g., Welsh et al, J Neurochem (1987) 49:846-851; the disclosure of which is herein incorporated by reference in its entirety).

In certain embodiments, the candidate compound is a modulator of the human GPR22 receptor according to the seventh aspect or a ligand of the human GPR22 receptor according to the eighth aspect.

In a twelfth aspect, the invention features a method of using a transgenic non-human mammal according to the tenth aspect to identify whether a candidate compound has efficacy for cardioprotection or neuroprotection in a mammal comprising the step of administering the candidate compound to the transgenic non-human mammal. In certain embodiments, said efficacy in the transgenic non-human mammal is indicative of efficacy in a mammal.

In certain embodiments, the candidate compound is a modulator or ligand of the human GPR22 receptor. In some embodiments, the candidate compound is not an endogenous ligand of the human GPR22 receptor.

Routes of administering the candidate compound to a non-human mammal are well known in the art and include but are not limited to oral, intraperitoneal, subcutaneous and intravenous administration.

In some embodiments, the dose of the compound is 0.1-100 mg/kg. In some embodiments, the dose is selected from the group consisting of 0.1 mg/kg, 0.3 mg/kg, 1.0 mg/kg, 3.0 mg/kg, 10 mg/kg, 30 mg/kg and 100 mg/kg.

Methods for identifying whether a candidate compound has efficacy for cardioprotection in a mammal are well known in the art (see, e.g., Fryer et al, Circ Res (1999) 84:846-851; Wang et al, J Pharmacol Toxicol Methods (2004) 50:163-174; the disclosure of each of which is herein incorporated by reference in its entirety). Methods for identifying whether a candidate compound has efficacy for neuroprotection in a mammal are well known in the art (see, e.g., Welsh et al, J Neurochem (1987) 49:846-851; the disclosure of which is herein incorporated by reference in its entirety).

In certain embodiments, the candidate compound is a modulator of the human GPR22 receptor according to the seventh aspect or a ligand of the human GP22 receptor according to the eighth aspect.

This application claims the benefit of priority from the following provisional patent application, filed via U.S. Express mail with the United States Patent and Trademark Office on the indicated date: U.S. Provisional Patent Application No. 60/727,203, filed Oct. 14, 2005. The disclosure of the foregoing provisional patent application is herein incorporated by reference in its entirety.

### BRIEF DESCRIPTION OF THE DRAWINGS

FIG. 1A is a schematic showing nucleotide sequence for wild-type human GPR22R425 coding region (SEQ ID NO:1). The codons that comprise the coding region are shown by alternate highlighting.

FIG. 1B is a schematic showing the amino acid sequence of human GPR22 R425 (SEQ ID NO:2; Genbank Accession No. NP_{—}005286) encoded by the nucleotide sequence of FIG. 1A.

FIG. 2A is a schematic showing nucleotide sequence for an exemplary expression-enhanced human GPR22R425 coding region (SEQ ID NO:3). The codons that comprise the coding region are shown by alternate highlighting.

Lower case letters indicate nucleotide substitutions by reference to SEQ ID NO:1. Unformatted lower case indicates substitution of an adenine or a thymine with a guanine or a cytosine. Italicized lower case indicates substitution of an adenine with a thymine or substitution of a thymine with an adenine. Bolded lower case indicates substitution of a guanine with a cytosine or substitution of a cytosine with a guanine. The unformatted nucleotide substitutions increase the GC-content of the coding region. The italicized and the bolded nucleotide substitutions do not change the GC-content of the coding region.

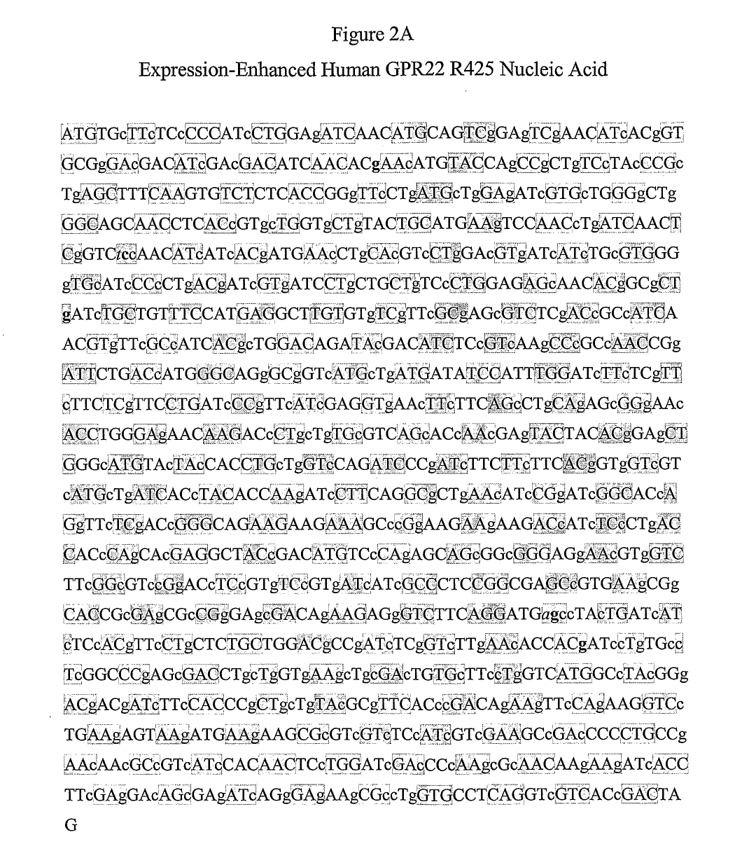

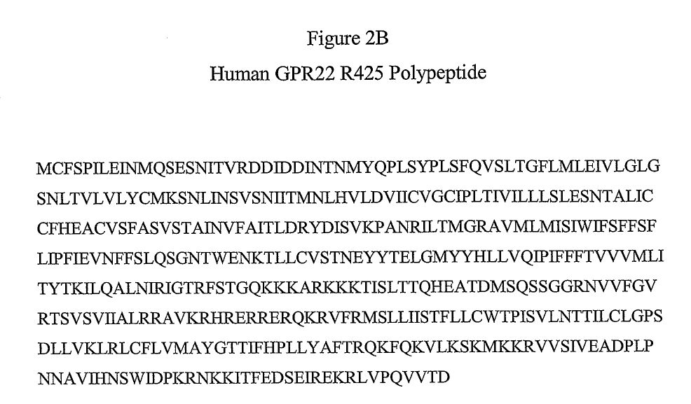

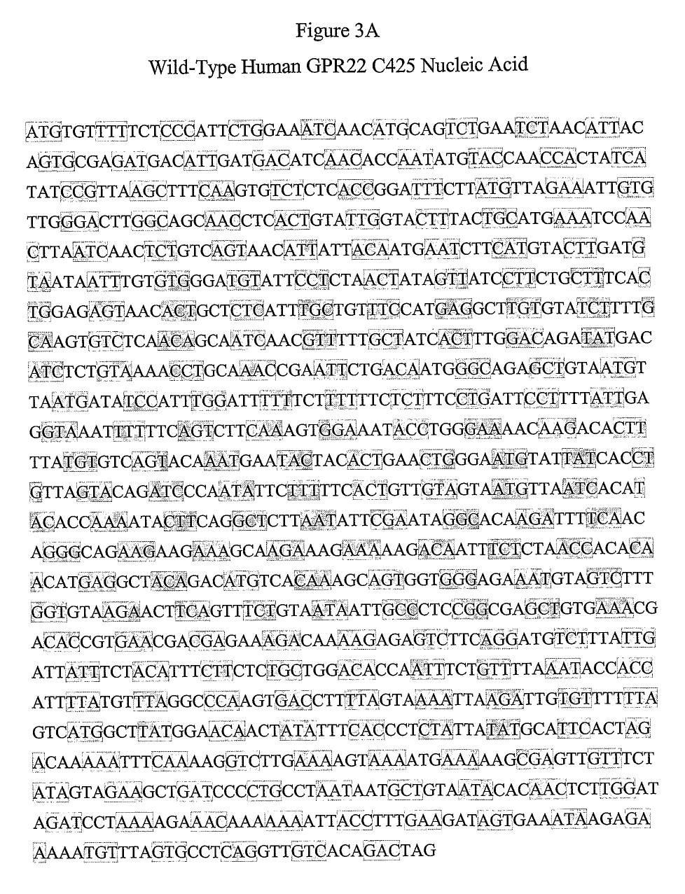

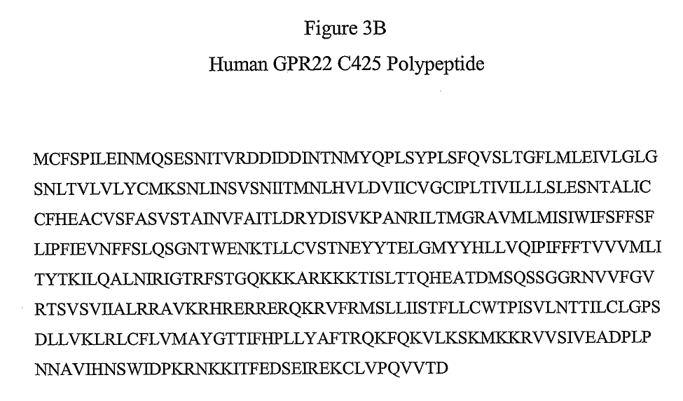

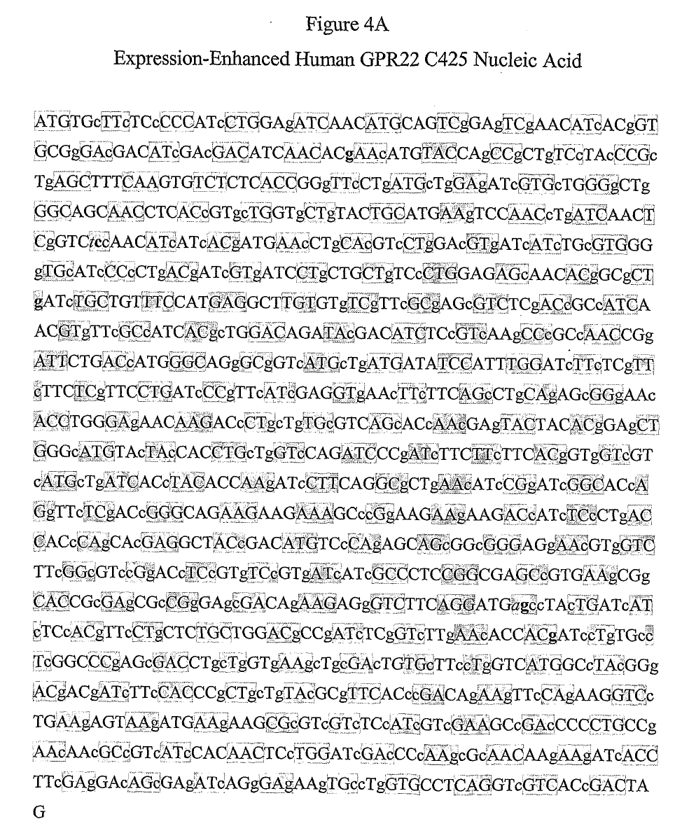

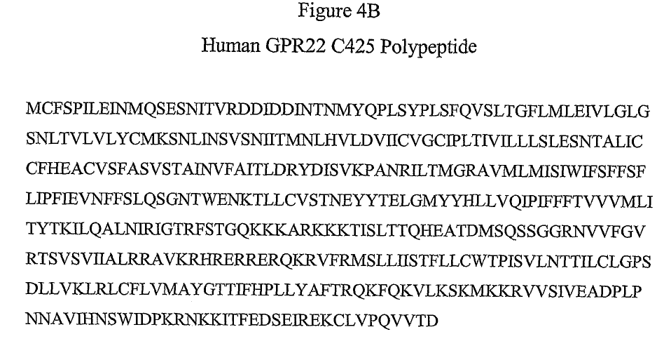

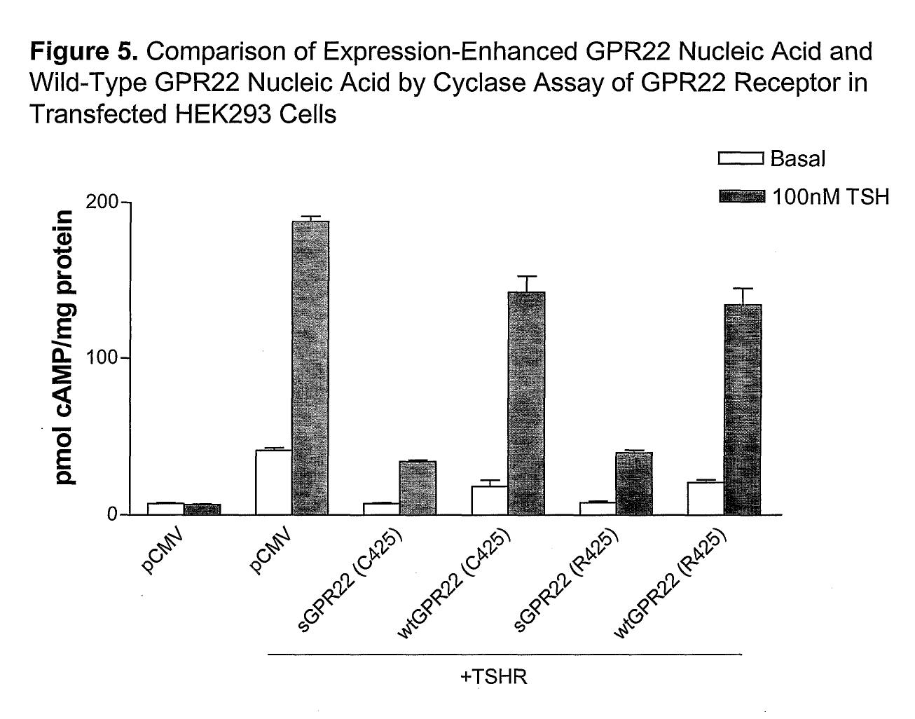

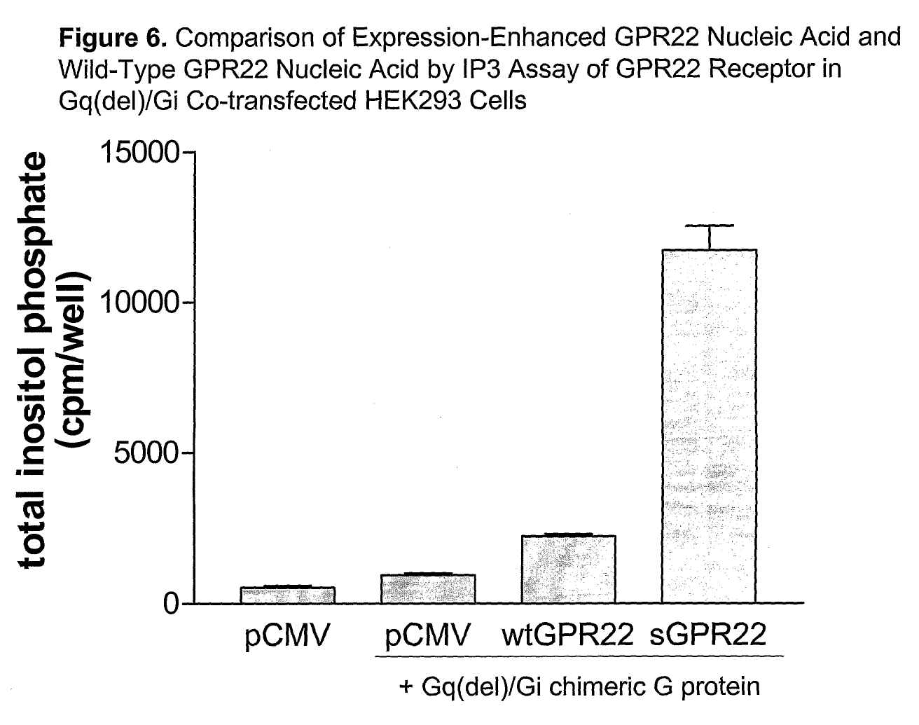

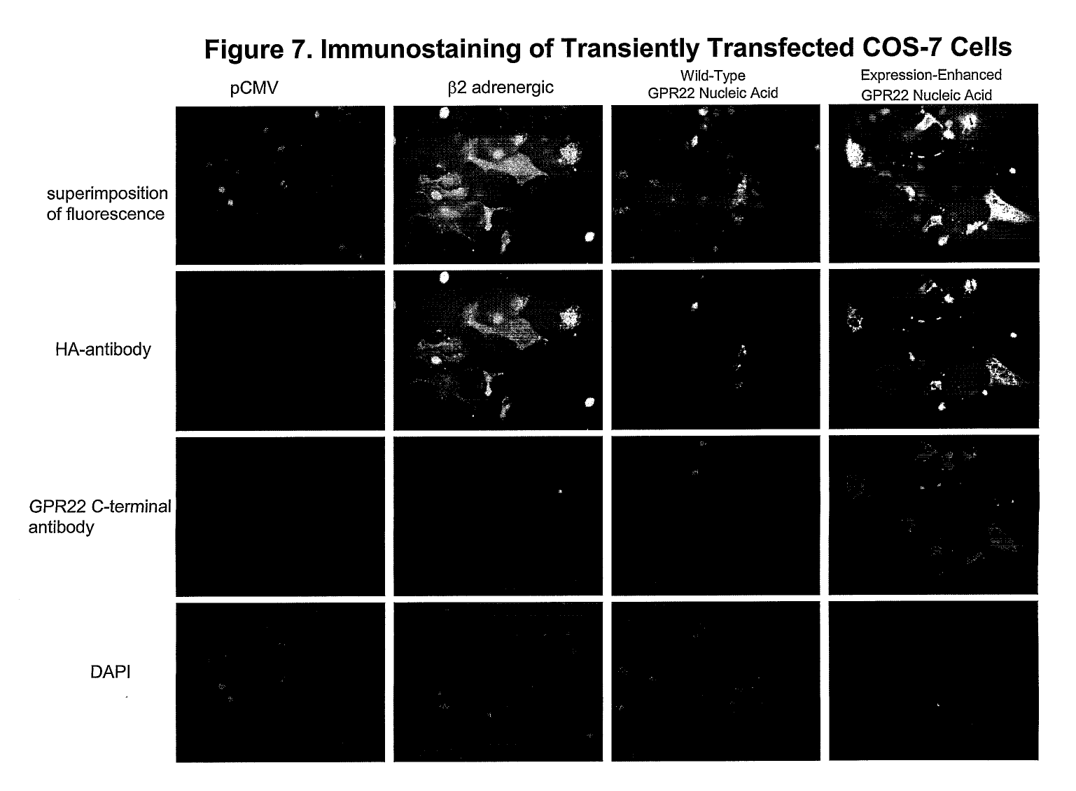

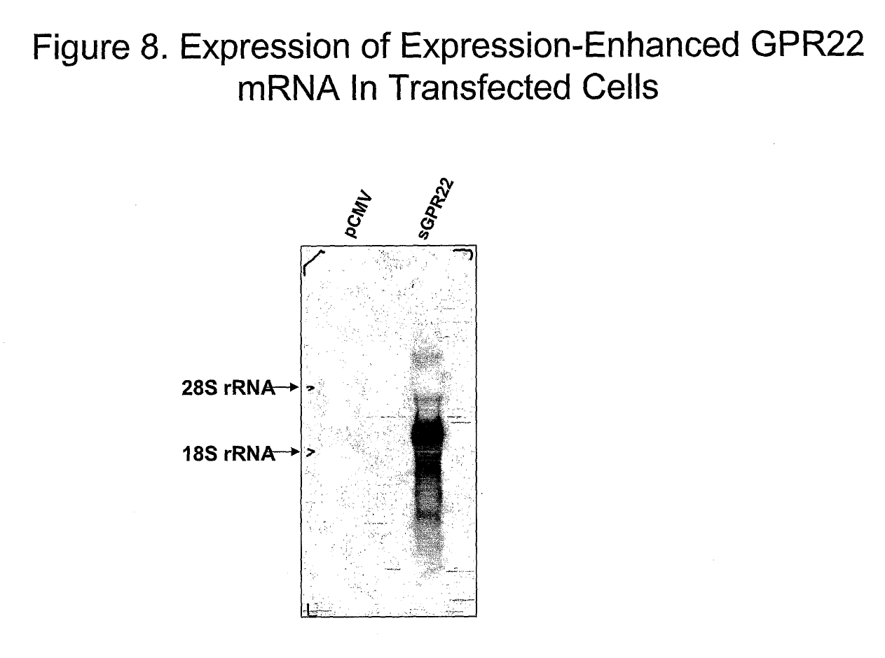

FIG. 2B is a schematic showing the amino acid sequence of human GPR22 R425 (SEQ ID NO:4) encoded by the nucleotide sequence of FIG. 2A.

FIG. 3A is a schematic showing nucleotide sequence for wild-type human GPR22C425 coding region (SEQ ID NO:5). The codons that comprise the coding region are shown by alternate highlighting.

FIG. 3B is a schematic showing the amino acid sequence of human GPR22 C425 (SEQ ID NO:6; Genbank Accession No. AAB63815) encoded by the nucleotide sequence of FIG. 3A.

FIG. 4A is a schematic showing nucleotide sequence for an exemplary expression-enhanced human GPR22C425 coding region (SEQ ID NO:7). The codons that comprise the coding region are shown by alternate highlighting.

Lower case letters indicate nucleotide substitutions by reference to SEQ ID NO:5. Unformatted lower case indicates substitution of an adenine or a thymine with a guanine or a cytosine. Italicized lower case indicates substitution of an adenine with a thymine or substitution of a thymine with an adenine. Bolded lower case indicates substitution of a guanine with a cytosine or substitution of a cytosine with a guanine. The unformatted nucleotide substitutions increase the GC-content of the coding region. The italicized and the bolded nucleotide substitutions do not change the GC-content of the coding region.

FIG. 4B is a schematic showing the amino acid sequence of human GPR22

C425 (SEQ ID NO:8) encoded by the nucleotide sequence of FIG. 4A.

FIG. 5 is a graph showing a comparison of expression-enhanced GPR22 nucleic acid and wild-type GPR22 nucleic acid by cyclase assay of GPR22 receptor in transfected HEK293 cells.

FIG. 6 is a graph showing a comparison of expression-enhanced GPR22 nucleic acid and wild-type GPR22 nucleic acid by IP₃ assay of GPR22 receptor in Gq(del)/Gi co-transfected HEK293 cells.

FIG. 7 is a series of photographs depicting immunostaining of transiently transfected COS-7 cells.

FIG. 8 is a Northern blot showing expression of expression-enhanced GPR22 mRNA in transfected cells.

### DETAILED DESCRIPTION OF THE INVENTION

The invention features methods for generating a GPR22 receptor-encoding nucleic acid providing for enhanced expression (e.g., an “expression-enhanced” GPR22 encoding nucleic acid) of the encoded GPR22 receptor polypeptide. The invention also features compositions, methods, and kits which take advantage of such GPR22 receptor-encoding nucleic acids for screening of modulators of GPR22 receptor. In certain preferred embodiments, the GPR22 receptor is a mammalian GPR22 receptor.

In further describing the subject invention, representative embodiments of the subject methods will be described first in greater detail, followed by a review of different applications in which the methods find use. In addition, compositions and kits that find use in certain embodiments of the subject methods will be described in greater detail.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Where an embodiment is drawn to a set of specific values (for example, percentages), it is expressly contemplated that each individual value (for example, percentage) or combination thereof is an additional, separate embodiment within the scope of the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a nucleotide” includes a plurality of such nucleotides and reference to “the codon” includes reference to one or more codons and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

Amino Acid Abbreviations used herein are set out in Table A:

TABLE A

| | | |
|---|---|---|
| alanine | Ala | A |
| arginine | Arg | R |
| asparagine | Asn | N |
| aspartic acid | Asp | D |
| cysteine | Cys | C |
| glutamic acid | Glu | E |
| glutamine | Gln | Q |
| glycine | Gly | G |
| histidine | His | H |
| isoleucine | Ile | I |
| leucine | Leu | L |
| lysine | Lys | K |
| methionine | Met | M |
| phenylalanine | Phe | F |
| proline | Pro | P |
| serine | Ser | S |
| threonine | Thr | T |
| tryptophan | Trp | W |
| tyrosine | Tyr | Y |
| valine | Val | V |

Nucleotide abbreviations as used herein are A (adenine), G (guanine), C (cytosine), and T (thymine).

The terms “polynucleotide” and “nucleic acid” are used interchangeably herein to refer to polymeric forms of nucleotides of any length. The polynucleotides of the invention generally contain deoxyribonucleotides or ribonucleotides, and may be generated recombinantly or synthetically. The term polynucleotide includes single-, double-stranded and triple helical molecules. Oligonucleotide generally refers to polynucleotides of between about 3 and about 100 nucleotides of single- or double-stranded DNA. However, for the purposes of this disclosure, there is no upper limit to the length of an oligonucleotide. Oligonucleotides are also known as oligomers or oligos and may be isolated from genes, or chemically synthesized by methods known in the art.

Throughout the specification, when reference is made to polynucleotides in terms a nucleotide sequence, such sequence is generally shown as a DNA sequence. It is to be understood that the invention also contemplates polynucleotides that are RNA, where exemplary RNA sequences can be readily derived from the exemplary DNA sequences provided herein by substituting thymine (T) with uracil (U).

The term “codon”, as used herein, refers to a set of three consecutive nucleotides in a strand of DNA or RNA that provides the genetic information to code for a specific amino acid which will be incorporated into a protein chain or serve as a termination signal.

The term “coding region”, as used herein, refers to a series of contiguous nucleotides in a strand of nucleic acid (DNA or RNA) that provides the genetic information for a polypeptide gene product.

The terms “polypeptide” and “protein”, used interchangeably herein, refer to a polymeric form of amino acids of any length, which amino acids in the context of the present invention are genetically encoded. The term includes fusion proteins, immunologically tagged proteins, and the like.

The term “endogenous” shall mean material that a vertebrate, for example a mammal, naturally produces. By way of illustration and not limitation, endogenous in reference to a GPR22 nucleic acid or to a GPR22 polypeptide shall mean a GPR22 nucleic acid or a GPR22 polypeptide which is naturally produced by a vertebrate, for example a mammal (for example, and not limitation, a human). As used herein, “endogenous” and “wild-type” are used interchangeably. By contrast, the term “non-endogenous in this context shall mean that which is not naturally produced by a vertebrate, for example a mammal (for example, and not limitation, a human).

The term “variant” shall mean a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. A typical variant of a polypeptide differs in amino acid sequence from another, reference polypeptide. A variant and reference polypeptide may differ in amino acid sequence by one or more substitutions, additions, deletions in any combination. A variant of a polynucleotide or polypeptide may be a naturally occurring one such as an allelic variant, or it may be a variant that is not known to occur naturally. Non-naturally occurring variants of polynucleotides and polypeptides may be made by mutagenesis techniques or by direct synthesis.

The term “expression-enhanced” as used herein refers to a first mammalian GPR22-encoding nucleic acid which has been modified so as to generate a non-endogenous substituted nucleic acid providing for enhanced expression of the encoded mammalian GPR22 polypeptide for the same type of host cell (e.g., a eukaryotic host cell, such as a mammalian or a melanophore host cell), wherein said enhanced expression is in comparison to the first nucleic acid or to a wild-type nucleic acid encoding the mammalian GPR22 receptor polypeptide.

An “expression vector” shall mean a DNA sequence that is required for the transcription of cloned DNA and translation of the transcribed mRNA in an appropriate host cell recombinant for the expression vector. An appropriately constructed expression vector should contain an origin of replication for autonomous replication in host cells, selectable markers, a limited number of useful restriction enzyme sites, a potential for high copy number, and active promoters. The cloned DNA to be transcribed is operably linked to a constitutively or conditionally active promoter within the expression vector.

A “host cell” shall mean a cell capable of having a vector incorporated therein. In the present context, the vector will typically contain nucleic acid encoding a GPR22 receptor in operable connection with a suitable promoter sequence to permit expression of the GPR22 receptor. In a preferred embodiment, the host cell is a eukaryotic cell, such as a mammalian cell, a yeast cell, or a melanophore cell.

The term “modulate” is intended to refer to an increase or decrease in the amount, quality, or effect of a particular activity, function or molecule.

A “GPR22 receptor modulator” is material, for example a ligand or compound, which modulates or changes an intracellular response when it binds to a GPR22 receptor.

“Directly identifying” or “directly identified”, in relationship to the phrase “candidate compound” or “test compound”, shall mean the screening of a candidate compound against a G protein-coupled receptor in the absence of a known ligand (e.g., a known agonist) to the G protein-coupled receptor.

An “agonist” is material, for example a ligand or compound, which by virtue of binding to a GPCR stimulates an intracellular response mediated by the GPCR. Agonist as used herein, and unless explicitly stated otherwise, encompasses full agonists and partial agonists.

A “partial agonist” is material, for example a ligand or compound, which by virtue of binding to a GPCR stimulates an intracellular response mediated by the GPCR, but to a lesser degree or extent than a full agonist.

An “inverse agonist” is material, for example a ligand or compound, which binds to a GPCR and which inhibits the baseline intracellular response initiated by the active form of the receptor below the normal base level activity which is observed in the absence of an agonist or partial agonist.

A “constitutively active GPCR” is a GPCR stabilized in an active state by means other than through binding of the receptor to an agonist or partial agonist. A constitutively active GPCR may be endogenous or non-endogenous.

An “antagonist” is material, for example a ligand or compound, which binds, and preferably binds competitively, to a GPCR at about the same site as an agonist or partial agonist but which does not activate an intracellular response initiated by the active form of the GPCR, and can thereby inhibit the intracellular response by agonist or partial agonist. An antagonist typically does not diminish the baseline intracellular response in the absence of an agonist or partial agonist.

A “modulator” as used herein in reference to a GPCR is material, for example a ligand or compound, which binds to the GPCR and stimulates or inhibits receptor functionality. A modulator of a GPCR shall be understood to encompass agonist, partial agonist, inverse agonist and antagonist as hereinbefore defined.

A “ligand” is a compound that specifically binds to a GPCR. An endogenous ligand is an endogenous compound that binds to an endogenous GPCR. A ligand of a GPCR can be, but is not limited to, an agonist, partial agonist, inverse agonist or antagonist of the GPCR as hereinbefore defined.

The terms “inhibit” or “inhibiting” in relationship to the term “response” shall mean that a response is decreased or prevented in the presence of a compound as opposed to in the absence of the compound.

“Stimulate” or “stimulating” in relationship to the term “response” shall mean that a response is increased in the presence of a compound as opposed to in the absence of the compound.

“Receptor functionality” shall refer herein to the normal operation of a GPCR to receive a stimulus and moderate an effect in the cell, including, but not limited to regulating gene transcription, regulating the influx or efflux of ions, effecting a catalytic reaction, and/or modulating activity through G proteins, such as eliciting a second messenger response.

The term “second messenger” shall be in reference to an intracellular response produced as a result of GPCR activation. Non-limiting examples of a second messenger include inositol 1,4,5-triphosphate (IP₃), diacylglycerol (DAG), cyclic AMP (cAMP), cyclic GMP (cGMP), MAP kinase activity, and Ca²⁺. Second messenger response can be measured for the identification of candidate compounds as, for example, inverse agonists, partial agonists, agonists, and antagonists of the receptor. In a particular embodiment, second messenger response can be measured for assessing a level of GPR22 receptor expression.

“Compound efficacy” shall mean a measurement of the ability of a compound to inhibit or stimulate receptor functionality, as opposed to receptor binding affinity. Exemplary means of measuring compound efficacy are disclosed in the Examples section of this patent document.

“Cell surface expression” as used herein in the context of a GPR22 polypeptide refers to the presence of the GPR22 polypeptide on the surface of a cell, e.g., to provide for a functional GPR22 which can be used in assays to identify agents that affect GPR22 activity.

A “small molecule” shall be taken to mean a compound having a molecular weight of less than about 10,000 grams per mole, including a peptide, peptidomimetic, amino acid, amino acid analogue, polynucleotide, polynucleotide analogue, nucleotide, nucleotide analogue, organic compound or inorganic compound (I.e. including a heterorganic compound or organometallic compound), and salts, esters and other pharmaceutically acceptable forms thereof. In certain preferred embodiments, small molecules are organic or inorganic compounds having a molecular weight of less than about 5,000 grams per mole. In certain preferred embodiments, small molecules are organic or inorganic compounds having molecular weight of less than about 1,000 grams per mole. In certain preferred embodiments, small molecules are organic or inorganic compounds having a molecular weight of less than about 500 grams per mole.

The terms “candidate compound” and “test compound,” used interchangeably herein, shall mean a molecule (for example, and not limitation, a chemical compound) that is amenable to a screening technique.

The terms “contact” or “contacting” mean bringing at least two moieties together, whether in an in vitro system or an in vivo system.

The term “pharmaceutical composition” shall mean a composition comprising at least one active ingredient, whereby the composition is amenable to investigation for a specified, efficacious outcome in a mammal (for example, and not limited to a human). Those of ordinary skill in the art will understand and appreciate the techniques appropriate for determining whether an active ingredient has a desired efficacious outcome, e.g., based upon the needs of the artisan.

The term “efficacy” as used herein in a therapeutic context with reference to a compound shall refer to the ability of the compound to elicit the biological or medicinal response in a tissue, system or individual that is being sought by a researcher, veterinarian, medical doctor or other clinician, which includes one or more of the following:
(1) Preventing the disease; for example, preventing a disease, condition or disorder in an individual that may be predisposed to the disease, condition or disorder but does not yet experience or display the pathology or symptomatology of the disease;
(2) Inhibiting the disease; for example, inhibiting a disease, condition or disorder in an individual that is experiencing or displaying the pathology or symptomatology of the disease, condition or disorder (i.e., arresting further development of the pathology and/or symptomatology);
(3) Ameliorating the disease; for example, ameliorating a disease, condition or disorder in an individual that is experiencing or displaying the pathology or symptomatology of the disease, condition or disorder (i.e., reversing the pathology and/or symptomatology); and
(4) Protecting against cardiomyocyte or neuronal cell death.

A “mammal” as used herein is intended to include, but not be limited to, mammalian farm animals, mammalian sport animals, mammalian pets, mice, rats, rabbits, dogs, cats, swine, cattle, sheep, horses, non-human primates, primates, and most preferably humans. In a preferred embodiment, a mammal is a human.

### Overview

Without being held to theory, the invention is based on the discovery that modifying the coding sequence of a mammalian GPR22-encoding nucleic acid can serve to enhance expression of the encoded polypeptide, including cell surface expression, even where the modifications do not change the amino acid sequence of the protein. In practicing the method of the invention, a coding region of a nucleic acid encoding a mammalian GPR22 polypeptide is analyzed to identify target nucleotides in the coding region sequence. A “target nucleotide” as used herein is an adenine or a thymine that can undergo a nucleotide substitution without changing an amino acid specified by the codon in which the target nucleotide is present. The target nucleotide is then substituted with another nucleotide without changing the amino acid specified by the codon to create a “substituted nucleic acid” comprising the mammalian GPR22 coding region, which provides for enhanced expression of the encoded GPR22 polypeptide. In preferred embodiments, the substituted nucleic acid is non-endogenous.

Accordingly, the invention provides methods for making a GPR22-encoding nucleic acid that provides for enhanced expression of the encoded GPR22 polypeptide (e.g., an “expression-enhanced” GPR22-encoding nucleic acid). The invention also features compositions, kits, and methods of use which take advantage of such GPR22-encoding nucleic acid (e.g., to screen for modulators of GP22 in cell-based assays).

In further describing the subject invention, representative embodiments of the subject methods will be described first in greater detail, followed by a review of different applications in which the methods find use. In addition, compositions and kits that find use in certain embodiments of the subject methods will be described in greater detail.

### Methods

As summarized above, the subject invention provides methods for modifying a first mammalian GPR22-encoding nucleic acid so as to generate a non-endogenous substituted nucleic acid encoding the mammalian GPR22 that provides for enhanced expression of the GPR22 polypeptide in a eukaryotic cell (such as a mammalian cell, a yeast cell or a melanophore cell). Accordingly, by expression-enhanced is meant an increase in expression (e.g., as measured by GPR22 receptor polypeptide expression or GPR22 receptor functional activity) of the GPR22 polypeptide encoded by the non-endogenous nucleic acid as compared to the expression of the GPR22 polypeptide encoded by the first nucleic acid or by a wild-type GPR22 nucleic acid in the same type of host cell (e.g., a eukaryotic host cell, such as a mammalian or a melanophore host cell). As will be explained in greater detail herein below, enhancement of expression can be assessed by comparing the expression of the GPR22 polypeptide encoded the first nucleic acid or by a wild-type nucleic acid with the expression of the GPR22 polypeptide encoded by the non-endogenous substituted GPR22 nucleic acid generated in accordance with the methods herein described. Said comparing may be by any suitable methods known in the art, and includes but is not limited here to cell based assays measuring the level of a second messenger, such as cyclic AMP (cAMP), cyclic GMP (cGMP), inositol triphosphate (IP₃), diacylglycerol (DAG), Ca²⁺, and MAP kinase activity.

In general, in practicing the subject methods, the coding region and the codons within the coding region, for a nucleic acid encoding a GP22 polypeptide are determined. After the codons have been identified, those nucleotides that are capable of undergoing substitution without changing the amino acid coded for by the codon, referred to herein as “target nucleotides”, are identified. One or more of the target nucleotides are then substituted with another nucleotide that preserves the encoded amino acid sequence. Any nucleotide may be substituted for a target nucleotide so long as by making such a substitution the amino acid specified by the codon is not changed, although in certain embodiments the substituted nucleotide is one that increases the GC-content of the coding region. In this manner, a non-endogenous substituted nucleic acid providing for enhanced expression of the encoded GPR22 polypeptide may be generated.

GPR22—Encoding Nucleic Acids

A GPR22-encoding nucleic acid of the subject invention comprises a nucleic acid sequence encoding the GPR22 polypeptide, preferably wherein the GPR22 is a mammalian GPR22. The disclosed methods are applicable to modifying the nucleic acid sequence encoding a GPR22 polypeptide derived from any mammalian species, including any and all naturally-occurring allelic variants. For example, and by no means limited hereby, a human (SEQ ID NO: 1 or SEQ ID NO: 5), a mouse (SEQ ID NO: 9), a rat (SEQ ID NO: 10), or a cow (SEQ ID NO: 11) nucleic acid encoding a GPR22 polypeptide can be modified to improve expression of the encoded GPR22 polypeptide in a recombinant host cell of choice, e.g., a recombinant mammalian or melanophore host cell. It is also understood that the starting material (e.g., a GPR22 nucleic acid) may be a naturally-occurring nucleic acid (e.g., a wild-type GPR22 nucleic acid sequence), recombinant nucleic acid, synthetic nucleic acid, or the like. Accordingly, any form of a mammalian GPR22-encoding nucleic acid may be modified in accordance with the methods herein described to provide enhanced expression of the encoded GPR22 polypeptide in a mammalian cell, so long as the amino acid sequence of the GPR22 polypeptide to be expressed is either known or capable of being known and is not changed by the modification to the underlying polynucleotide sequence encoding that polypeptide. It is expressly contemplated that a GPR22-encoding nucleic acid of the subject invention comprising a nucleic acid sequence encoding the GPR22 polypeptide, preferably wherein the GPR22 is a mammalian GPR22, may further comprise 5′ and/or 3′ untranslated nucleotide sequence. It is also expressly contemplated that a GPR22-encoding nucleic acid of the subject invention comprising a nucleic acid sequence encoding the GPR22 polypeptide, preferably wherein the GPR22 is a mammalian GPR22, may encode a fusion protein comprising the encoded GPR22 polypeptide.

In one embodiment of particular interest, the nucleic acid to be modified encodes an allelic variant of the human GPR22 polypeptide. There are several well known allelic variants of the human GPR22 polypeptide. Two such variants are GPR22R425 and GPR22C425 (human GPR22 receptor polypeptide comprising arginine or cysteine residue, respectively, at amino acid position 425). Exemplary amino acid sequence for GPR22R425 and for GPR22C425 are well known and are set forth in the appendix to this specification at SEQ ID NOs: 2 and 6, respectively. The GPR22 amino acid sequence of GenBank Accession No. AAI07129 is presented by way of further exemplification and not limitation of wild-type human GPR22C425 amino acid sequence, differing from SEQ ID NO: 6 in comprising a glycine residue instead of a valine residue at amino acid position 53 of SEQ ID NO: 6. However, although the invention is described with respect to modifying the coding region of a nucleic acid encoding a GPR22R425 or GPR22C425 polypeptide so as to enhance expression (e.g., as assessed by increased polypeptide expression or by increased functional activity) of the encoded polypeptide as compared to the observed expression of the polypeptide encoded by the wild-type nucleic acid sequence (i.e., SEQ ID NOs: 1 and 5 respectfully), it is understood that the disclosed methods are equally applicable to modifying any nucleic acid variant encoding a GPR22 polypeptide so as to provide for enhanced expression of the encoded GPR22 polypeptide.

Accordingly, it is expressly contemplated that in certain embodiments a “mammalian GPR22 receptor” is an endogenous mammalian GPR22 receptor. By way of illustration and not limitation, a “mammalian GPR22 receptor” that is an endogenous mammalian GPR22 receptor encompasses human GPR22 receptor of SEQ ID NO: 2, human GPR22 receptor of SEQ ID NO: 6, mouse GPR22 receptor of SEQ ID NO:10, rat GPR22 receptor of SEQ ID NO: 12, and cow GPR22 receptor of SEQ ID NO: 14. In certain embodiments, a “mammalian GPR22 receptor” is an endogenous mammalian GPR22 receptor selected from the group consisting of human GPR22 receptor of SEQ ID NO: 2, human GPR22 receptor of SEQ ID NO: 6, mouse GPR22 receptor of SEQ ID NO: 10, rat GPR22 receptor of SEQ ID NO: 12, and cow GPR22 receptor of SEQ ID NO: 14.

It is further expressly contemplated that in certain embodiments a “mammalian GPR22 receptor” is a GPCR having at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9% identity to an endogenous mammalian GPR22 receptor. It is further expressly contemplated that in certain embodiments a “mammalian GPR22 receptor” is a GPCR having at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9% identity to a mammalian GPR22 receptor polypeptide selected from the group consisting of SEQ ID NO: 2, SEQ ID NO: 6, SEQ ID NO: 10, SEQ ID NO:12 and SEQ ID NO: 14. In certain embodiments, the GPCR having at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9% identity to an endogenous mammalian GPR22 receptor or to a mammalian GPR22 receptor polypeptide selected from the group consisting of SEQ ID NO: 2, SEQ ID NO: 6, SEQ ID NO: 10, SEQ ID NO:12 and SEQ ID NO: 14 is an endogenous GPCR. In certain embodiments, the GPCR having at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9% identity to an endogenous mammalian GPR22 receptor or to a mammalian GPR22 receptor polypeptide selected from the group consisting of SEQ ID NO: 2, SEQ ID NO: 6, SEQ ID NO: 10, SEQ ID NO:12 and SEQ ID NO: 14 is a non-endogenous GPCR. In certain embodiments, the GPCR having at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9% identity to an endogenous mammalian GPR22 receptor or to a mammalian GPR22 receptor polypeptide selected from the group consisting of SEQ ID NO: 2, SEQ ID NO: 6, SEQ ID NO: 10, SEQ ID NO: 12 and SEQ ID NO: 14 can be shown to promote (to increase) cardiomyocyte survival. In certain embodiments, the GPCR having at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9% identity to an endogenous mammalian GPR22 receptor or to a mammalian GPR22 receptor polypeptide selected from the group consisting of SEQ ID NO: 2, SEQ ID NO: 6, SEQ ID NO: 10, SEQ ID NO: 12 and SEQ ID NO: 14 can be shown to rescue cardiomyocytes from apoptosis (to decrease cardiomyocyte apoptosis). In certain embodiments, the GPCR having at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9% identity to an endogenous mammalian GPR22 receptor or to a mammalian GPR22 receptor polypeptide selected from the group consisting of SEQ ID NO: 2, SEQ ID NO: 6, SEQ ID NO: 10, SEQ ID NO:12 and SEQ ID NO: 14 exhibits detectable constitutive activity. In certain embodiments, the GPCR having at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9% identity to an endogenous mammalian GPR22 receptor or to a mammalian GPR22 receptor polypeptide selected from the group consisting of SEQ ID NO: 2, SEQ ID NO: 6, SEQ ID NO: 10, SEQ ID NO:12 and SEQ ID NO: 14 exhibits detectable constitutive activity for lowering a level of intracellular cAMP. In certain embodiments, the GPCR having at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9% identity to an endogenous mammalian GPR22 receptor or to a mammalian GPR22 receptor polypeptide selected from the group consisting of SEQ ID NO: 2, SEQ ID NO: 6, SEQ ID NO: 10, SEQ ID NO:12 and SEQ ID NO: 14 and exhibiting detectable constitutive activity for lowering a level of intracellular cAMP couples to Gi. In certain embodiments, the GPCR having at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9% identity to an endogenous mammalian GPR22 receptor or to a mammalian GPR22 receptor polypeptide selected from the group consisting of SEQ ID NO: 2, SEQ ID NO: 6, SEQ ID NO: 10, SEQ ID NO:12 and SEQ ID NO: 14 specifically binds an antibody that recognizes an endogenous mammalian GPR22 receptor (an antibody that recognizes an endogenous mammalian GPR22 receptor can be obtained commercially from, e.g., ABR-Affinity BioReagents, Golden, Colo.; GeneTex, San Antonio, Tex.; and Novus Biologicals, Littleton, Colo.) or specifically binds a known ligand of an endogenous mammalian GPR22 receptor. In certain embodiments, the known ligand of the endogenous mammalian GPR22 receptor is a known modulator of the endogenous mammalian GPR22 receptor. In certain embodiments, the known ligand of the endogenous mammalian GPR22 receptor is an endogenous ligand of the endogenous mammalian GPR22 receptor.

It is further expressly contemplated that in certain embodiments a “mammalian GPR22 receptor” is a GPCR having at least about 75%, at least about 80%, at least about 85%, at least about 90%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9% identity to the endogenous human GPR22 receptor of SEQ ID NO: 2 or SEQ ID NO: 6. In certain embodiments, the GPCR having at least about 75%, at least about 80%, at least about 85%, at least about 90%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9% identity to the endogenous human GPR22 receptor of SEQ ID NO: 2 or SEQ ID NO: 6 is an endogenous GPCR. In certain embodiments, the GPCR having at least about 75%, at least about 80%, at least about 85%, at least about 90%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9% identity to the endogenous human GPR22 receptor of SEQ ID NO: 2 or SEQ ID NO: 6 is a non-endogenous GPCR. In certain embodiments, the GPCR having at least about 75%, at least about 80%, at least about 85%, at least about 90%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9% identity to the endogenous human GPR22 receptor of SEQ ID NO: 2 or SEQ ID NO: 6 can be shown to promote (to increase) cardiomyocyte survival. In certain embodiments, the GPCR having at least about 75%, at least about 80%, at least about 85%, at least about 90%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9% identity to the endogenous human GPR22 receptor of SEQ ID NO: 2 or SEQ ID NO: 6 can be shown to rescue cardiomyocytes from apoptosis (to decrease cardiomyocyte apoptosis). In certain embodiments, the GPCR having at least about 75%, at least about 80%, at least about 85%, at least about 90%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9% identity to the endogenous human GPR22 receptor of SEQ ID NO: 2 or SEQ ID NO: 6 exhibits detectable constitutive activity. In certain embodiments, the GPCR having at least about 75%, at least about 80%, at least about 85%, at least about 90%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9% identity to the endogenous human GPR22 receptor of SEQ ID NO: 2 or SEQ ID NO: 6 exhibits detectable constitutive activity for lowering a level of intracellular cAMP. In certain embodiments, the GPCR having at least about 75%, at least about 80%, at least about 85%, at least about 90%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9% identity to the endogenous human GPR22 receptor of SEQ ID NO: 2 or SEQ ID NO: 6 and exhibiting detectable constitutive activity for lowering a level of intracellular cAMP couples to Gi. In certain embodiments, the GPCR having at least about 75%, at least about 80%, at least about 85%, at least about 90%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9% identity to the endogenous human GPR22 receptor of SEQ ID NO: 2 or SEQ ID NO: 6 specifically binds an antibody that recognizes an endogenous mammalian GPR22 receptor (an antibody that recognizes an endogenous mammalian GPR22 receptor can be obtained commercially from, e.g., ABR-Affinity BioReagents, Golden, Colo.; GeneTex, San Antonio, Tex.; and Novus Biologicals, Littleton, Colo.) or specifically binds a known ligand of an endogenous mammalian GPR22 receptor. In certain embodiments, the known ligand of the endogenous mammalian GPR22 receptor is a known modulator of the endogenous mammalian GP 22 receptor. In certain embodiments, the known ligand of the endogenous mammalian GPR22 receptor is an endogenous ligand of the endogenous mammalian GPR22 receptor.

It is further expressly contemplated that in certain embodiments, a “human GPR22 receptor” is a GPCR having at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9% identity, or 100% identity to SEQ ID NO: 2 or to SEQ ID NO: 6. In certain embodiments, a “mouse GPR22 receptor” is a GPCR having at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9% identity, or 100% identity to SEQ ID NO: 10. In certain embodiments, a “rat GPR22 receptor” is a GPCR having at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9% identity, or 100% identity to SEQ ID NO: 12. In certain embodiments, a “cow GPR22 receptor” is a GPCR having at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9% identity, or 100% identity to SEQ ID NO: 14. In certain embodiments, a “human GPR22 receptor” is a GPCR derived from SEQ ID NO: 2 or SEQ ID NO: 6 by substitution, deletion or addition of one or several amino acids in the amino acid sequence of SEQ ID NO: 2 or SEQ ID NO: 6. In certain embodiments, a “mouse GPR22 receptor” is a GPCR derived from SEQ ID NO: 10 by substitution, deletion or addition of one or several amino acids in the amino acid sequence of SEQ ID NO: 10. In certain embodiments, a “rat GPR22 receptor” is a GPCR derived from SEQ ID NO: 12 by substitution, deletion or addition of one or several amino acids in the amino acid sequence of SEQ ID NO: 12. In certain embodiments, a “cow GPR22 receptor” is a GPCR derived from SEQ ID NO: 14 by substitution, deletion or addition of one or several amino acids in the amino acid sequence of SEQ ID NO: 14. In certain embodiments, a “human GPR22 receptor” is a GPCR derived from SEQ ID NO: 2 or SEQ ID NO: 6 by no more than 10 conservative amino acid substitutions and/or no more than 3 non-conservative amino acid substitutions in the amino acid sequence of SEQ ID NO: 2 or SEQ ID NO: 6. In certain embodiments, a “mouse GPR22 receptor” is a GPCR derived from SEQ ID NO: 10 by no more than 10 conservative amino acid substitutions and/or no more than 3 non-conservative amino acid substitutions in the amino acid sequence of SEQ ID NO: 10. In certain embodiments, a “rat GPR22 receptor” is a GPCR derived from SEQ ID NO: 12 by no more than 10 conservative amino acid substitutions and/or no more than 3 non-conservative amino acid substitutions in the amino acid sequence of SEQ ID NO: 12. In certain embodiments, a “cow GPR22 receptor” is a GPCR derived from SEQ ID NO: 14 by no more than 10 conservative amino acid substitutions and/or no more than 3 non-conservative amino acid substitutions in the amino acid sequence of SEQ ID NO: 14. In certain embodiments, arginine, lysine and histidine may conservatively substitute for each other; glutamic acid and aspartic acid may conservatively substitute for each other; glutamine and asparagine may conservatively substitute for each other; leucine, isoleucine and valine may conservatively substitute for each other; phenylalanine, tryptophan and tyrosine may conservatively substitute for each other; and glycine, alanine, serine, threonine and methionine may conservatively substitute for each other. The amino acid substitutions, amino acid deletions, and amino acid additions may be at any position (e.g., the C- or N-terminus, or at internal positions). In certain embodiments, the “human GPR22 receptor”, the “mouse GPR22 receptor”, the “rat GPR22 receptor”, or the “cow GPR22 receptor” is an endogenous GPCR. In certain embodiments, the “human GPR22 receptor”, the “mouse GPR22 receptor”, the “rat GPR22 receptor”, or the “cow GPR22 receptor” is a non-endogenous GPCR. In certain embodiments, the “human GPR22 receptor”, the “mouse GPR22 receptor”, the “rat GPR22 receptor”, or the “cow GPR22 receptor” can be shown to promote (to increase) cardiomyocyte survival. In certain embodiments, the “human GPR22 receptor”, the “mouse GPR22 receptor”, the “rat GPR22 receptor”, or the “cow GPR22 receptor” can be shown to rescue cardiomyocytes from apoptosis (to decrease cardiomyocyte apoptosis). In certain embodiments, the “human GPR22 receptor”, the “mouse GPR22 receptor”, the “rat GPR22 receptor”, or the “cow GPR22 receptor” exhibits detectable constitutive activity. In certain embodiments, the “human GPR22 receptor”, the “mouse GPR22 receptor”, the “rat GPR22 receptor”, or the “cow GPR22 receptor” exhibits detectable constitutive activity for lowering a level of intracellular cAMP. In certain embodiments, the “human GPR22 receptor”, the “mouse GPR22 receptor”, the “rat GPR22 receptor”, or the “cow GPR22 receptor” exhibits detectable constitutive activity for lowering a level of intracellular cAMP and couples to Gi. In certain embodiments, the “human GPR22 receptor”, the “mouse GPR22 receptor”, the “rat GPR22 receptor”, or the “cow GPR22 receptor” specifically binds an antibody that recognizes an endogenous mammalian GPR22 receptor (an antibody that recognizes an endogenous mammalian GPR22 receptor can be obtained commercially from, e.g., ABR-Affinity BioReagents, Golden, Colo.; GeneTex, San Antonio, Tex.; and Novus Biologicals, Littleton, Colo.) or specifically binds a known ligand of an endogenous mammalian GPR22 receptor. In certain embodiments, the known ligand of the endogenous mammalian GPR22 receptor is a known modulator of the endogenous mammalian GPR22 receptor. In certain embodiments, the known ligand of the endogenous mammalian GPR22 receptor is an endogenous ligand of the endogenous mammalian GPR22 receptor.

In certain embodiments, percent identity is evaluated using the Basic Local Alignment Search Tool (“BLAST”), which is well known in the art [See, e.g., Karlin and Altschul, Proc Natl Acad Sci USA (1990) 87:2264-2268; Altschul et al., J Mol Biol (1990) 215:403-410; Altschul et all, Nature Genetics (1993) 3:266-272; and Altschul et al., Nucleic Acids Res (1997) 25:3389-3402; the disclosure of each of which is herein incorporated by reference in its entirety]. The BLAST programs may be used with the default parameters or with modified parameters provided by the user. Preferably, the parameters are default parameters.

A preferred method for determining the best overall match between a query sequence (e.g., the amino acid sequence of SEQ ID NO:2 or 6) and a sequence to be interrogated, also referred to as a global sequence alignment, can be determined using the FASTDB computer program based on the algorithm of Brutlag et al. [Comp App Biosci (1990) 6:237-245; the disclosure of which is herein incorporated by reference in its entirety]. In a sequence alignment the query and interrogated sequences are both amino acid sequences. The results of said global sequence alignment is in percent identity. Preferred parameters used in a FASTDB amino acid alignment are: Matrix=PAM 0, k-tuple=2, Mismatch Penalty=1, Joining Penalty-20, Randomization Group=25, Length=0, Cutoff Score=1, Window Size=sequence length, Gap Penalty=5, Gap Size Penalty=0.05, Window Size=247 or the length of the interrogated amino acid sequence, whichever is shorter.

If the interrogated sequence is shorter than the query sequence due to N- or C-terminal deletions, not because of internal deletions, the results, in percent identity, must be manually corrected because the FASTDB program does not account for N- and C-terminal truncations of the interrogated sequence when calculating global percent identity. For interrogated sequences truncated at the N- and C-termini, relative to the query sequence, the percent identity is corrected by calculating the number of residues of the query sequence that are N- and C-terminal of the interrogated sequence, that are not matched/aligned with a corresponding interrogated sequence residue, as a percent of the total amino acids of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the interrogated sequence, which are not matched/aligned with the query sequence, are considered for the purposes of manually adjusting the percent identity score. That is, only query amino acid residues outside the farthest N- and C-terminal residues of the interrogated sequence.

For example, a 90 amino acid residue interrogated sequence is aligned with a 100-residue query sequence to determine percent identity. The deletion occurs at the N-terminus of the interrogated sequence and therefore, the FASTDB alignment does not match/align with the first residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched, the final percent identity would be 90%.

In another example, a 90-residue interrogated sequence is compared with a 100-residue query sequence. This time the deletions are internal so there are no residues at the N- or C-termini of the interrogated sequence, which are not matched/aligned with the query. In this case, the percent identity calculated by FASTDB is not manually corrected. Once again, only residue positions outside the N- and C-terminal ends of the subject sequence, as displayed in the FASTDB alignment, which are not matched/aligned with the query sequence are manually corrected. No other corrections are made for the purposes of the present invention.

It is further expressly contemplated that in certain embodiments a “mammalian GPR22 receptor” is a GPCR encoded by a polynucleotide hybridizing at high stringency to the complement of a wild-type polynucleotide encoding a mammalian GPR22 receptor polypeptide or to the complement of a polynucleotide selected from the group consisting of SEQ ID NO: 1, SEQ ID NO: 5, SEQ ID NO: 9, SEQ ID NO: 11 and SEQ ID NO: 13. In certain embodiments, the GPCR encoded by a polynucleotide hybridizing at high stringency to the complement of a wild-type polynucleotide encoding a mammalian GPR22 receptor polypeptide or to the complement of a polynucleotide selected from the group consisting of SEQ ID NO: 1, SEQ ID NO: 5, SEQ ID NO: 9, SEQ ID NO: 11 and SEQ ID NO: 13 is an endogenous GPCR. In certain embodiments, the GPCR encoded by a polynucleotide hybridizing at high stringency to the complement of a wild-type polynucleotide encoding a mammalian GPR22 receptor polypeptide or to the complement of a polynucleotide selected from the group consisting of SEQ ID NO: 1, SEQ ID NO: 5, SEQ ID NO: 9, SEQ ID NO: 11 and SEQ ID NO: 13 is a non-endogenous GPCR. In certain embodiments, the GPCR encoded by a polynucleotide hybridizing at high stringency to the complement of a wild-type polynucleotide encoding a mammalian GPR22 receptor polypeptide or to the complement of a polynucleotide selected from the group consisting of SEQ ID NO: 1, SEQ ID NO: 5, SEQ ID NO: 9, SEQ ID NO: 11 and SEQ ID NO: 13 can be shown to promote (to increase) cardiomyocyte survival. In certain embodiments, the GPCR encoded by a polynucleotide hybridizing at high stringency to the complement of a wild-type polynucleotide encoding a mammalian GPR22 receptor polypeptide or to the complement of a polynucleotide selected from the group consisting of SEQ ID NO: 1, SEQ ID NO: 5, SEQ ID NO: 9, SEQ ID NO: 11 and SEQ ID NO: 13 can be shown to rescue cardiomyocytes from apoptosis (to decrease cardiomyocyte apoptosis). In certain embodiments, the GPCR encoded by a polynucleotide hybridizing at high stringency to the complement of a wild-type polynucleotide encoding a mammalian GPR22 receptor polypeptide or to the complement of a polynucleotide selected from the group consisting of SEQ ID NO: 1, SEQ ID NO: 5, SEQ ID NO: 9, SEQ ID NO: 11 and SEQ ID NO: 13 exhibits detectable constitutive activity. In certain embodiments, the GPCR encoded by a polynucleotide hybridizing at high stringency to the complement of a wild-type polynucleotide encoding a mammalian GPR22 receptor polypeptide or to the complement of a polynucleotide selected from the group consisting of SEQ ID NO: 1, SEQ ID NO: 5, SEQ ID NO: 9, SEQ ID NO: 11 and SEQ ID NO: 13 exhibits detectable constitutive activity for lowering a level of intracellular cAMP. In certain embodiments, the GPCR encoded by a polynucleotide hybridizing at high stringency to the complement of a wild-type polynucleotide encoding a mammalian GPR22 receptor polypeptide or to the complement of a polynucleotide selected from the group consisting of SEQ ID NO: 1, SEQ ID NO: 5, SEQ ID NO: 9, SEQ ID NO: 11 and SEQ ID NO: 13 and exhibiting detectable constitutive activity for lowering a level of intracellular cAMP couple to Gi. In certain embodiments, the GPCR encoded by a polynucleotide hybridizing at high stringency to the complement of a wild-type polynucleotide encoding a mammalian GPR22 receptor polypeptide or to the complement of a polynucleotide selected from the group consisting of SEQ ID NO: 1, SEQ ID NO: 5, SEQ ID NO: 9, SEQ ID NO: 11 and SEQ ID NO: 13 specifically binds an antibody that recognizes an endogenous mammalian GPR22 receptor (an antibody that recognizes an endogenous mammalian GPR22 receptor can be obtained commercially from, e.g., ABR-Affinity BioReagents, GeneTex, and Novus Biologicals) or specifically binds a known ligand of an endogenous mammalian GPR22 receptor. In certain embodiments, the known ligand of the endogenous mammalian GPR22 receptor is a known modulator of the endogenous mammalian GPR22 receptor. In certain embodiments, the known ligand of the endogenous mammalian GPR22 receptor is an endogenous ligand of the endogenous mammalian GPR22 receptor. Hybridization techniques are well known to the skilled artisan. In some embodiments, stringent hybridization conditions include overnight incubation at 42° C. in a solution comprising: 50% formamide, 5×SSC (1×SSC=150 mM NaCl, 15 mM trisodium citrate), 50 mM sodium phosphate (pH 7.6), 5×Denhardt's solution, 10% dextran sulfate, and 20 μg/ml denatured, sheared salmon sperm DNA; followed by washing the filter in 0.1×SSC at about 65° C.

It is expressly contemplated that the claims may be drafted to exclude any proper subset of “mammalian GPR22 receptor.”

It is to be understood that the invention contemplates both DNA and RNA polynucleotides. However, throughout the specification, and solely for clarity and convenience, the nucleotide sequences, and guidance as to the substitutions to be made according to the invention, are provided in terms of deoxyribonucleotides (e.g., the sequences provided herein are DNA sequences). Exemplary sequences for RNA molecules, as well as guidance as to modifications to generate a RNA molecule of the invention, can be readily derived from the present disclosure by substituting thymine (T) with uracil (U).

A first step in the subject methods is to determine the coding region of a nucleic acid encoding a GPR22 polypeptide. This may be done by means well known in the art. For instance, this may be done by isolating, amplifying and sequencing a nucleic acid encoding a GPR22 polypeptide of interest obtained from a selected mammalian sample (e.g., a human). In this manner, the genetic sequence of the nucleic acid and the amino acid sequence of the encoded protein may be derived and the translation start and stop sites determined, thereby determining the coding region.

Alternatively, the coding region of a nucleic acid encoding a GPR22 polypeptide may be determined by reference to a known nucleic acid sequence encoding a GPR22 polypeptide, such as those published in Genbank. For example, SEQ ID NO: 1 represents a wild-type nucleic acid sequence encoding human GPR22R425 polypeptide, determined by reference, e.g., to Genbank accession number NM_{—}005295. SEQ ID NO: 5 represents a wild-type nucleic acid sequence encoding human GPR22C425 polypeptide, determined by reference, e.g., to Genbank accession number AC002381.1.

Once the coding region for the nucleic acid encoding the GPR22 polypeptide of interest has been ascertained, the codons encoding the GPR22 polypeptide can be determined. Due to the degeneracy of the genetic code, codons often allow for variations in the nucleotide sequence without changing the amino acid sequence of the encoded protein. Since each codon consists of three nucleotides, and the nucleotides comprising DNA are restricted to four nucleotides (A, C, G or T), there are 64 possible combinations of nucleotides, 61 of which encode amino acids (the remaining three codons encode signals ending translation). The “genetic code” which shows which codons encode which amino acids is reproduced herein as Table 1. As a result, many amino acids are designated by more than one codon. For example, the amino acids alanine and proline are coded for by four different codons and serine and arginine by six codons each, whereas tryptophan and methionine are coded by just one triplet codon. This degeneracy allows for DNA base compositions to vary over a wide range without altering the amino acid sequence of the proteins encoded by the DNA. Accordingly for purposes of the subject invention, although the amino acid sequence encoded by the GPR22 nucleic acid to be modified is held to be fixed, the nucleotides that comprise the codons encoding those amino acids are subject to change.

TABLE 1

| | T | C | A | G |
|---|---|---|---|---|
| T | TTT Phe (F) | TCT Ser (S) | TAT Tyr (Y) | TGT Cys (C) |
| | TTC Phe (F) | TCC Ser (S) | TAC | TGC |
| | TTA Leu (L) | TCA Ser (S) | TAA Ter | TGA Ter |
| | TTG Leu (L) | TCG Ser (S) | TAG Ter | TGG Trp (W) |
| C | CTT Leu (L) | CCT Pro (P) | CAT His (H) | CGT Arg (R) |
| | CTC Leu (L) | CCC Pro (P) | CAC His (H) | CGC Arg (R) |
| | CTA Leu (L) | CCA Pro (P) | CAA Gln (Q) | CGA Arg (R) |
| | CTG Leu (L) | CCG Pro (P) | CAG Gln (Q) | CGG Arg (R) |
| A | ATT Ile (I) | ACT Thr (T) | AAT Asn (N) | AGT Ser (S) |
| | ATC Ile (I) | ACC Thr (T) | AAC Asn (N) | AGC Ser (S) |
| | ATA Ile (I) | ACA Thr (T) | AAA Lys (K) | AGA Arg (R) |
| | ATG Met (M) | ACG Thr (T) | AAG Lys (K) | AGG Arg (R) |
| G | GTT Val (V) | GCT Ala (A) | GAT Asp (D) | GGT Gly (G) |
| | GTC Val (V) | GCC Ala (A) | GAC Asp (D) | GGC Gly (G) |
| | GTA Val (V) | GCA Ala (A) | GAA Glu (E) | GGA Gly (G) |
| | GTG Val (V) | GCG Ala (A) | GAG Glu (E) | GGG Gly (G) |

Accordingly, an expression-enhanced nucleic acid sequence of the invention encoding a GPR22 polypeptide can be generated by identifying target nucleotides within the codons that are capable of undergoing substitution with another nucleotide without changing the underlying amino acid sequence. This can be done using any method known in the art, for instance, an expression-enhanced GPR22 nucleic acid sequence can be designed manually using a standard Genetic Code Table, as that depicted in Table 1, to define the various codons used for any given amino acid and substituting at least one nucleotide within the codon with a different nucleotide. Alternatively, this can be accomplished by using computer-assisted methods, such as, for example, the Wisconsin Genetics Computer Group back translation software package available from Accelrys, Inc., San Diego, Calif. Additionally, various other algorithms and computer software programs for generating an expression-enhanced substituted nucleic acid according to the invention encoding a GPR22 polypeptide are readily available to those of ordinary skill in the art, see, e.g. the “EditSeq” function in the Lasergene Package, available from DNAstar, Inc., Madison, Wis., and the backtranslation function in the Vector NTI Suite, available from InforMax, Inc., Bethesda, Md.

Any nucleotide may be substituted for a target nucleotide so long as by making the substitution the amino acid specified by the codon is not changed. For convenience's sake, the nucleotide that replaces the target nucleotide is referred to herein as the “substitute nucleotide” and the resulting modified nucleic acid is referred to herein as a “substituted nucleic acid.” For instance, if the amino acid coded for by a specified codon is leucine and the first two nucleotides of the codon are CT (i.e., not a TT), then an A, C, G, or T nucleotide in the third position of the codon may be exchanged with any other nucleotide, without changing the underlying amino acid coded for. See Table 1. Thus, if the target nucleotide in the leucine codon is A, the substitute nucleotide can be C, G or T. If the target nucleotide in the leucine codon is T, the substitute nucleotide can be C, G or A. It is noted, that although for purposes of this example the substituted nucleotide was in position 3 of the codon, this is in no way limiting. Any nucleotide in any position (1, 2 or 3) of the codon may be exchanged one for another so long as the encoded amino acid is not changed. For example, both TTG and CTG codons encode leucine. Further, more than one target nucleotide in a codon may be substituted; for example, both TTA and CTG encode leucine. A codon in which a target nucleotide is replaced with a substitute nucleotide is referred to herein as a “modified codon”. As will be understood by those of ordinary skill in the art, the methods of the invention can be applied such that the distribution of modified codons in the coding sequence can vary significantly, with the proviso that the encoded amino acid sequence remains the same.

It is expressly contemplated that the method for modifying a nucleic acid encoding a mammalian GPR22 amino acid sequence so as to create a non-endogenous substituted nucleic acid comprising at least one substitute nucleotide providing for enhanced expression of the encoded GPR22 polypeptide is within the scope of the invention. However, it should be noted that although a substituted nucleic acid (DNA or RNA) will differ in sequence from the unmodified nucleic acid from which it is derived, e.g. a wild-type GPR22 nucleic acid, the GPR22 amino acid sequence encoded by the unmodified and substituted nucleic acids will be the same.

In one embodiment of particular interest, the substitute nucleotide is one that increases the GC-content of the coding region (and thus decreases the AT-content of the coding region). In another embodiment, the substitute nucleotide is one that maintains the GC-content of the coding region (and thus maintains the AT-content of the coding region).

In one embodiment, target nucleotides of particular interest are those present in a coding region in a sequence of at least three or more contiguous nucleotides that are less desired in the coding region. For example, including but not limited to the case where it is desirable to reduce the AT-content of a coding region, target nucleotides of particular interest for substitution are As or Ts present in a contiguous sequence of three or more As or Ts (e.g., ATAT), or in a contiguous sequence of three or more As (e.g., AAAA), or a contiguous sequence of three or more Ts (e.g., TTTT). In this manner an expression-enhanced nucleic acid sequence comprising a mammalian GPR22 coding region may be designed. In other related embodiments, the target nucleotide(s) are present in a sequence of 3, 4, 5, 6, 7, 8, 9, 10 or more As and/or Ts.

Although nucleotide substitutions in the codons of the coding region of a nucleic acid encoding a GPR22 polypeptide are made for the purpose of enhancing expression, this in no way implies that in generating a nucleic acid providing for enhanced expression of GPR22 in accordance with the subject invention all possible target nucleotides must be replaced with a substitute nucleotide. That is, generation of a nucleic acid providing for enhanced expression of GPR22 in accordance with the subject invention does not require that every nucleotide capable of being changed within a codon or every codon capable of being changed within the coding region be changed. In one embodiment, the GC-content is increased. For instance, the GC-content of the coding region can be increased by at least about 10%, by at least about 15%, by at least about 20%, by at least about 25%, by at least about 30%, by at least about 35%, by at least about 40%, by at least about 45%, by at least about 50%, by at least about 55%, by at least about 60%, or at least about 65%, or more. In certain embodiments, the GC-content of the coding region of the substituted nucleic acid encoding the mammalian GPR22 receptor amino acid sequence is at least about 35%, at least about 36%, at least about 37%, at least about 38%, at least about 39%, at least about 40%, at least about 45%, at least about 50%, at least about 55%, or at least about 60%.

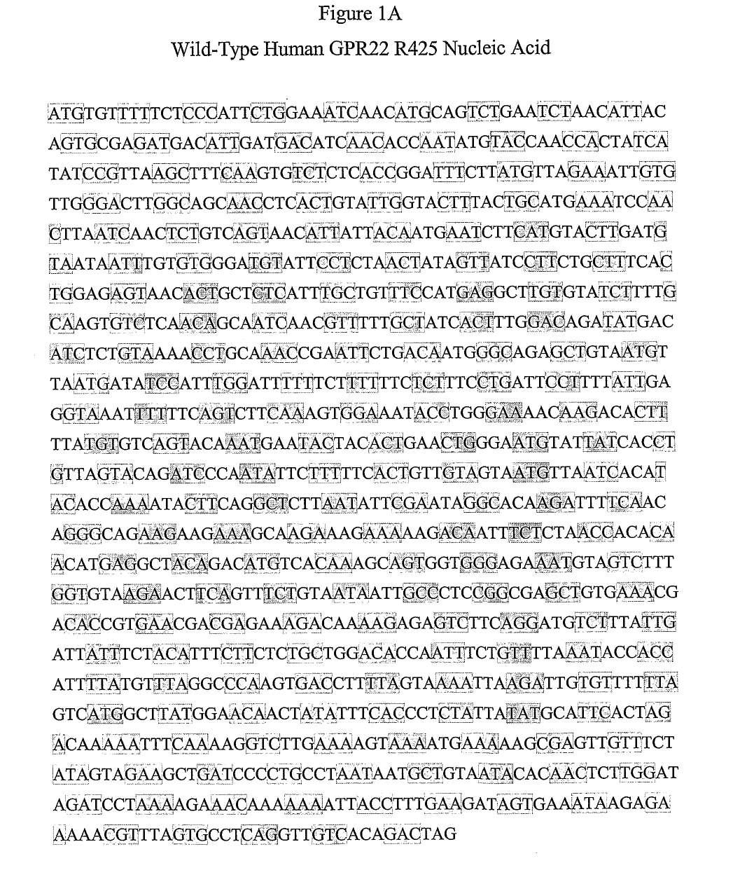

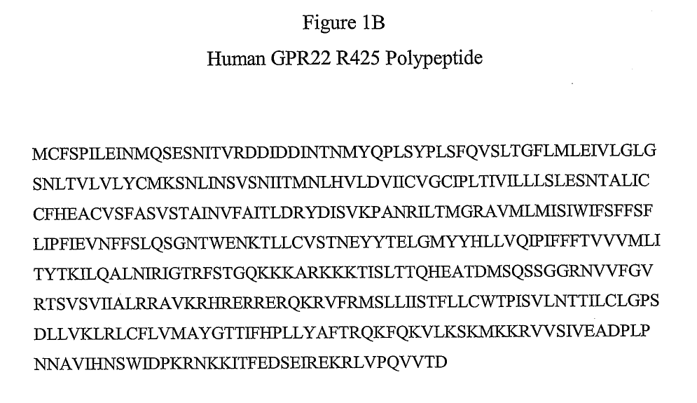

In one non-limiting embodiment, as seen in FIG. 2A, a substituted human GPR22R425 polynucleotide is provided wherein the GC-content is increased by at least 60%, specifically by about 64%. Specifically, wild-type GPR22R425 nucleotide sequence of FIG. 1A has a GC-content of about 36%, whereas the GC-content of the substituted GPR22R425 nucleotide sequence derived therefrom (FIG. 2A) is about 59%, which is equivalent to about a 64% increase in GC-content. In another non-limiting embodiment, as seen in FIG. 4A, a substituted human GPR22C425 polynucleotide is provided wherein the GC-content is increased by at least about 60%, specifically by about 64%. Specifically, wild-type GPR22C425 nucleotide sequence of FIG. 3A has a GC-content of about 36%, where as the GC-content of the substituted GPR22C425 nucleotide sequence derived therefrom (FIG. 4A) is about 59%, which is equivalent to about a 64% increase in GC-content. As can be seen with reference to FIGS. 2A and 4A, the nucleotide substitutions in the substituted GPR22R425 sequence are identical to those substitutions in the substituted GPR22C425 sequence, however, the encoded protein is different by one amino acid at position 425, where in GPR22 R425 the amino acid at position 425 is an arginine (R), and in GPR22C425 the amino acid at position 425 is a cysteine (C). This is due to a C/T nucleotide polymorphism at nucleotide position 1273, which is responsible for the R/C amino acid polymorphism at amino acid position 425.

It is to be noted that the above two substituted nucleic acids are set forth for exemplification purposes only, and this is not to be construed as limiting the scope of the subject invention in any way. There are 433 codons (excluding the stop codon) in GP22 R425 and GPR22C425. In the exemplified substituted GPR22R425 and GPR22C425 polynucleotides, 278 of the 433 codons (i.e., about 64%) have at least one nucleotide substitution which changes (increases) the GC-content of the nucleic acid. An additional three codons were modified without changing the GC-content. Sixteen codons comprising a target nucleotide for which a substitution could have been made to change (e.g., increase) the GC-content of the nucleic acid were not modified (the codons encoding phenylalanine at amino acid position 40 of SEQ ID NOs:2 and 6, glutamine at position 41, serine at positions 43 and 380, histidine at position 118, alanine at position 120, cysteine at position 121, arginine at positions 140 and 296, isoleucine at positions 151 and 162 and 164, leucine at position 235, lysine at position 253, glutamic acid at position 391, and proline at position 428), such that about 95% of the codons comprising a target nucleotide for which a substitution could have been made to change (e.g., increase) the GC-content were modified. The resultant substituted construct evidences an enhancement in GPR22 receptor expression (as seen in FIGS. 5-7). Accordingly, any nucleotide position not substituted in FIG. 2A or 4A need not be substituted in order to provide for enhanced expression, although such substitutions may have a further positive effect and would therefore be within the scope of the present invention. In SEQ ID NOs: 3 and 7, there are 302 nucleotide substitutions; 295 of the nucleotide substitutions change the GC-content (unformatted lower case in FIGS. 2A and 4A), whereas 7 of the nucleotide substitutions do not (italicized or bolded lower case in FIGS. 2A and 4A; e.g., nucleotide positions 229 and 943 in FIGS. 2A and 54A). It is expressly contemplated that as few as 1 or 2 nucleotide positions may be substituted and result in enhanced expression (whether or not such a substitution changes the GC-content or AT-content of the nucleic acid) and would, therefore, be within the scope of the present invention.

Accordingly, deviations from strict adherence to a complete optimization may also be made, for example: (i) to accommodate the introduction or maintenance of restriction sites, (ii) disrupt undesirable nucleotide stretches, (iii) to provide or maintain PCR amplification sites, and the like.

In one embodiment, an A (adenine) or T (thymine) nucleotide in at least about 10% of the codons of a coding region for a nucleic acid encoding a GPR22 polypeptide is substituted with a G (guanine) or C (cytosine) nucleotide. In another embodiment, an A or T nucleotide in at least about 15% of the codons of a coding region for a nucleic acid encoding a GPR22 polypeptide is substituted with a G or C nucleotide. In another embodiment, an A or T nucleotide in at least about 20% of the codons of a coding region for a nucleic acid encoding a GPR22 polypeptide is substituted with a G or C nucleotide. In another embodiment, an A or T nucleotide in at least about 25% of the codons of a coding region for a nucleic acid encoding a GPR22 polypeptide is substituted with a G or C nucleotide. In another embodiment, an A or T nucleotide in at least about 30% of the codons of a coding region for a nucleic acid encoding a GPR22 polypeptide is substituted with a G or C nucleotide. In another embodiment, an A or T nucleotide in at least about 35% of the codons of a coding region for a nucleic acid encoding a GPR22 polypeptide is substituted with a G or C nucleotide. In another embodiment, an A or T nucleotide in at least about 40% of the codons of a coding region for a nucleic acid encoding a GPR22 polypeptide is substituted with a G or C nucleotide. In another embodiment, an A or T nucleotide in at least about 45% of the codons of a coding region for a nucleic acid encoding a GPR22 polypeptide is substituted with a G or C nucleotide. In one embodiment, an A or T nucleotide in at least about 50% of the codons of a coding region for a nucleic acid encoding a GPR22 polypeptide is substituted for a G or C nucleotide. In another embodiment, an A or T nucleotide in at least about 55% of the codons of a coding region for a nucleic acid encoding a GPR22 polypeptide is substituted with a G or C nucleotide. In one embodiment, an A or T nucleotide in at least about 60% of the codons of a coding region for a nucleic acid encoding a GPR22 polypeptide is substituted for a G or C nucleotide. It is to be noted, however, that as only one codon, ATG, encodes methionine, neither the A nor the T in this codon can be changed without changing the encoded amino acid. Similarly, only one codon, TGG, encodes tryptophan such that the T cannot be changed without changing the encoded amino acid.

In one embodiment, an A (adenine) or T (thymine) nucleotide in at least about 10% of the codons of a coding region for a nucleic acid encoding a GPR22 polypeptide which comprise a target nucleotide for which a substitution can be made to increase the GC-content is substituted with a G (guanine) or C (cytosine) nucleotide. In one embodiment, an A or T nucleotide in at least about 20% of the codons of a coding region for a nucleic acid encoding a GPR22 polypeptide which comprise a target nucleotide for which a substitution can be made to increase the GC-content is substituted with a G or C nucleotide. In one embodiment, an A or T nucleotide in at least about 30% of the codons of a coding region for a nucleic acid encoding a GPR22 polypeptide which comprise a target nucleotide for which a substitution can be made to increase the GC-content is substituted with a G or C nucleotide. In one embodiment, an A or T nucleotide in at least about 40% of the codons of a coding region for a nucleic acid encoding a GPR22 polypeptide which comprise a target nucleotide for which a substitution can be made to increase the GC-content is substituted with a G or C nucleotide. In one embodiment, an A or T nucleotide in at least about 50% of the codons of a coding region for a nucleic acid encoding a GP22 polypeptide which comprise a target nucleotide for which a substitution can be made to increase the GC-content is substituted with a G or C nucleotide. In one embodiment, an A or T nucleotide in at least about 60% of the codons of a coding region for a nucleic acid encoding a GPR22 polypeptide which comprise a target nucleotide for which a substitution can be made to increase the GC-content is substituted with a G or C nucleotide. In one embodiment, an A or T nucleotide in at least about 70% of the codons of a coding region for a nucleic acid encoding a GPR22 polypeptide which comprise a target nucleotide for which a substitution can be made to increase the GC-content is substituted with a G or C nucleotide. In one embodiment, an A or T nucleotide in at least about 75% of the codons of a coding region for a nucleic acid encoding a GPR22 polypeptide which comprise a target nucleotide for which a substitution can be made to increase the GC-content is substituted with a G or C nucleotide. In one embodiment, an A or T nucleotide in at least about 80% of the codons of a coding region for a nucleic acid encoding a GPR22 polypeptide which comprise a target nucleotide for which a substitution can be made to increase the GC-content is substituted with a G or C nucleotide. In one embodiment, an A or T nucleotide in at least about 85% of the codons of a coding region for a nucleic acid encoding a GPR22 polypeptide which comprise a target nucleotide for which a substitution can be made to increase the GC-content is substituted with a G or C nucleotide. In one embodiment, an A or T nucleotide in at least about 90% of the codons of a coding region for a nucleic acid encoding a GPR22 polypeptide which comprise a target nucleotide for which a substitution can be made to increase the GC-content is substituted with a G or C nucleotide. In one embodiment, an A or T nucleotide in at least about 95% of the codons of a coding region for a nucleic acid encoding a GPR22 polypeptide which comprise a target nucleotide for which a substitution can be made to increase the GC-content is substituted with a G or C nucleotide. In one embodiment, an A or T nucleotide in 100% of the codons of a coding region for a nucleic acid encoding a GPR22 polypeptide which comprise a target nucleotide for which a substitution can be made to increase the GC-content is substituted with a G or C nucleotide.

In one embodiment, an A (adenine) nucleotide in at least about 10% of the codons of a coding region for a nucleic acid encoding a GPR22 polypeptide which comprise a target nucleotide which is an A is substituted with a T (thymine), G (guanine) or C (cytosine) nucleotide. In one embodiment, an A nucleotide in at least about 20% of the codons of a coding region for a nucleic acid encoding a GPR22 polypeptide which comprise a target nucleotide which is an A is substituted with a T, G or C nucleotide. In one embodiment, an A nucleotide in at least about 30% of the codons of a coding region for a nucleic acid encoding a GPR22 polypeptide which comprise a target nucleotide which is an A is substituted with a T, G or C nucleotide. In one embodiment, an A nucleotide in at least about 40% of the codons of a coding region for a nucleic acid encoding a GPR22 polypeptide which comprise a target nucleotide which is an A is substituted with a T, G or C nucleotide. In one embodiment, an A nucleotide in at least about 50% of the codons of a coding region for a nucleic acid encoding a GPR22 polypeptide which comprise a target nucleotide which is an A is substituted with a T, G or C nucleotide. In one embodiment, an A nucleotide in at least about 60% of the codons of a coding region for a nucleic acid encoding a GPR22 polypeptide which comprise a target nucleotide which is an A is substituted with a T, G or C nucleotide. In one embodiment, an A nucleotide in at least about 70% of the codons of a coding region for a nucleic acid encoding a GPR22 polypeptide which comprise a target nucleotide which is an A is substituted with a T, G or C nucleotide. In one embodiment, an A nucleotide in at least about 75% of the codons of a coding region for a nucleic acid encoding a GPR22 polypeptide which comprise a target nucleotide which is an A is substituted with a T, G or C nucleotide. In one embodiment, an A nucleotide in at least about 80% of the codons of a coding region for a nucleic acid encoding a GPR22 polypeptide which comprise a target nucleotide which is an A is substituted with a T, G or C nucleotide. In one embodiment, an A nucleotide in at least about 85% of the codons of a coding region for a nucleic acid encoding a GPR22 polypeptide which comprise a target nucleotide which is an A is substituted with a T, G or C nucleotide. In one embodiment, an A nucleotide in at least about 90% of the codons of a coding region for a nucleic acid encoding a GPR22 polypeptide which comprise a target nucleotide which is an A is substituted with a T, G or C nucleotide. In one embodiment, an A nucleotide in at least about 95% of the codons of a coding region for a nucleic acid encoding a GPR22 polypeptide which comprise a target nucleotide which is an A is substituted with a T, G or C nucleotide. In one embodiment, an A nucleotide in 100% of the codons of a coding region for a nucleic acid encoding a GPR22 polypeptide which comprise a target nucleotide which is an A is substituted with a T, G or C nucleotide.

In one embodiment, a T (thymine) nucleotide in at least about 10% of the codons of a coding region for a nucleic acid encoding a GPR22 polypeptide which comprise a target nucleotide which is a T is substituted with an A (adenine), G (guanine) or C (cytosine) nucleotide. In one embodiment, a T nucleotide in at least about 20% of the codons of a coding region for a nucleic acid encoding a GPR22 polypeptide which comprise a target nucleotide which is a T is substituted with an A, G or C nucleotide. In one embodiment, a T nucleotide in at least about 30% of the codons of a coding region for a nucleic acid encoding a GPR22 polypeptide which comprise a target nucleotide which is a T is substituted with an A, G or C nucleotide. In one embodiment, a T nucleotide in at least about 40% of the codons of a coding region for a nucleic acid encoding a GPR22 polypeptide which comprise a target nucleotide which is a T is substituted with an A, G or C nucleotide. In one embodiment, a T nucleotide in at least about 50% of the codons of a coding region for a nucleic acid encoding a GPR22 polypeptide which comprise a target nucleotide which is a T is substituted with an A, G or C nucleotide. In one embodiment, a T nucleotide in at least about 60% of the codons of a coding region for a nucleic acid encoding a GPR22 polypeptide which comprise a target nucleotide which is a T is substituted with an A, G or C nucleotide. In one embodiment, a T nucleotide in at least about 70% of the codons of a coding region for a nucleic acid encoding a GPR22 polypeptide which comprise a target nucleotide which is a T is substituted with an A, G or C nucleotide. In one embodiment, a T nucleotide in at least about 75% of the codons of a coding region for a nucleic acid encoding a GPR22 polypeptide which comprise a target nucleotide which is a T is substituted with an A, G or C nucleotide. In one embodiment, a T nucleotide in at least about 80% of the codons of a coding region for a nucleic acid encoding a GPR22 polypeptide which comprise a target nucleotide which is a T is substituted with an A, G or C nucleotide. In one embodiment, a T nucleotide in at least about 85% of the codons of a coding region for a nucleic acid encoding a GPR22 polypeptide which comprise a target nucleotide which is a T is substituted with an A, G or C nucleotide. In one embodiment, a T nucleotide in at least about 90% of the codons of a coding region for a nucleic acid encoding a GPR22 polypeptide which comprise a target nucleotide which is a T is substituted with an A, G or C nucleotide. In one embodiment, a T nucleotide in at least about 95% of the codons of a coding region for a nucleic acid encoding a GPR22 polypeptide which comprise a target nucleotide which is a T is substituted with an A, G or C nucleotide. In one embodiment, a T nucleotide in 100% of the codons of a coding region for a nucleic acid encoding a GPR22 polypeptide which comprise a target nucleotide which is a T is substituted with an A, G or C nucleotide. For instance, in one embodiment, the wild-type nucleic acid sequence of SEQ ID NO: 1, encoding the GPR22R425 protein of SEQ ID NO: 2, is used as a template for designing a non-endogenous expression-enhanced nucleic acid sequence encoding the GPR22R425 protein. In a specific example, the substituted nucleic acid is that of SEQ ID NO: 3, which encodes the GPR22R425 polypeptide of SEQ ID NO: 4. It is to be noted that although SEQ ID NOs: 1 and 3 differ from one another, the encoded proteins (i.e., SEQ ID NOs: 2 and 4) do not (i.e., SEQ ID NO: 2 is identical to SEQ ID NO: 4). In another embodiment, the wild-type nucleic acid sequence of SEQ ID NO: 5 encoding the GPR22C425 protein of SEQ ID NO: 6 is used as a template for designing a non-endogenous expression-enhanced nucleic acid sequence encoding the GPR22C425 protein. In a specific example, the substituted nucleic acid is that of SEQ ID NO: 7, which encodes the GPR22C425 polypeptide of SEQ ID NO: 8. It is to be noted that although SEQ ID NOs: 5 and 7 differ from one another, the encoded polypeptides (i.e., SEQ ID NOs: 6 and 8) do not (i.e., SEQ ID NO: 6 is identical to SEQ ID NO: 8).

A comparison of the wild-type and substituted coding sequences of the GPR22 R425 and GPR22C425 proteins is shown in FIGS. 1-4. These Figures demonstrate that many nucleotides within the GPR22R425 and GPR22C425 wild-type coding regions for which substitution could be made without changing the amino acid sequence of the encoded GPR22 polypeptide were altered to provide the substituted nucleic acids exemplified in these Figures. For the substituted nucleic acids exemplified in these Figures, codon modification according to the invention resulted in an increased GC-content and a decreased AT(U)-content.

Synthesizing a GPR22—Encoding Nucleic Acid

Following the design of a substituted GPR22 nucleic acid providing for enhanced expression of the encoded GPR22 polypeptide, preferably wherein the substituted GPR22 nucleic acid is non-endogenous, a number of options are available for synthesizing the substituted nucleic acid using standard and routine molecular biological manipulations well known to those of ordinary skill in the art. For instance, and not to be limited hereby, a wild-type GPR22 encoding nucleic acid may be used as a template, and the substituted nucleic acid constructed by subjecting the template nucleic acid sequence to site directed mutagenesis using a Transformer Site-Directed™ Mutagenesis Kit (Clontech) or a QuickChange™ Site-Directed™ Mutagenesis Kit (Stratagene), both according to the manufacturer instructions. In this manner, any undesired target nucleotides may be substituted with any of those nucleotides that are more desired, so long as by making such a substitution the amino acid specified by the codon is not changed.

Alternatively, once designed, a full length expression-enhanced GPR22 encoding nucleic acid can be constructed by first synthesizing a series of complementary oligonucleotide pairs of about 80-90 nucleotides each in length that span the entire length of the desired sequence using standard methods. These oligonucleotide pairs are synthesized such that upon annealing, they form double stranded fragments of 80-90 base pairs, containing cohesive ends, e.g., each oligonucleotide in the pair is synthesized to extend 3, 4, 5, 6, 7, 8, 9, 10, or more bases beyond the region that is complementary to the other oligonucleotide in the pair. The single-stranded ends of each pair of oligonucleotides are designed to anneal with the single-stranded end of another pair of oligonucleotides. The oligonucleotide pairs are allowed to anneal, and approximately five to six of these double-stranded fragments are then allowed to anneal together via the cohesive single stranded ends, and then they are ligated together and cloned into a standard bacterial cloning vector.

The construct is then sequenced by standard methods. Several of these constructs consisting of 5 to 6 fragments of 80 to 90 base pair fragments ligated together, i.e., fragments of about 500 base pairs, are prepared, such that the entire desired sequence is represented in a series of plasmid constructs. The inserts of these plasmids are then cut with appropriate restriction enzymes and ligated together to form the final synthetic construct. The final construct is then cloned into a vector, and sequenced to confirm sequence identity. Additional methods would be immediately apparent to the skilled artisan. In addition, gene synthesis is readily available commercially. See for instance: J. Cello, A. V. Paul, E. Wimmer, Science 297, 1016 (2002). Hence, in using the described methods, an entire polypeptide sequence or fragment, variant, or derivative thereof may be expression-enhanced. Various desired fragments, variants or derivatives are designed, and each is then expression-enhanced individually.

Vectors and Host Cells

Once a non-endogenous substituted GPR22 nucleic acid providing for enhanced expression of the encoded GPR22 polypeptide has been designed and generated, the substituted nucleic acid can be cloned into a vector and operatively linked to appropriate regulatory sequence(s), a promoter, a terminator sequence, and the like, by methods well known in the art, such as those described below. The vector so generated may be used to genetically modify a host cell of interest and the expression levels of the encoded protein may then be assessed. Hence, in one embodiment, the invention is directed to polynucleotide expression constructs, vectors, and host cells comprising a substituted GPR22 nucleic acid providing for enhanced expression of the encoded GPR22 polypeptide or fragment thereof.

Accordingly, the invention provides vectors (also referred to as “constructs”) comprising a substituted GPR22 nucleic acid providing for enhanced expression of the encoded GPR22 polypeptide. In many embodiments of the invention, the substituted GPR22 nucleic acid providing for enhanced expression of the encoded GPR22 polypeptide is operably linked to an expression control sequence, such as a promoter that directs the transcription of GPR22 mRNA using the substituted nucleic acid as template.

Suitable promoters can be any promoter that is functional in a eukaryotic host cell, including viral promoters and promoters derived from eukaryotic genes. Further suitable promoters can be any promoter that is functional in an animal host cell (e.g., an insect, mammal, fish, amphibian, bird or reptile host cell), including viral promoters and promoters derived from animal genes. Exemplary promoters include, but are not limited to, the following: the promoter of the mouse metallothionein I gene sequence (Hamer et al., J. Mol. Appl. Gen. 1:273-288, 1982); the TK promoter of Herpes virus (McKnight, Cell 31:355-365, 1982); the SV40 early promoter (Benoist et al., Nature (London) 290:304-310, 1981); the yeast gall gene sequence promoter (Johnston et al., Proc. Natl. Acad. Sci. (USA) 79:6971-6975, 1982); the CMV promoter, the EF-1 promoter, ecdysone-responsive promoter(s), tetracycline-responsive promoter, and the like. Viral promoters may be of particular interest as they are generally particularly strong promoters. Promoters for use in the present invention are selected such that they are functional in the cell type (and/or mammal) into which they are being introduced.

Furthermore, substituted GPR22 nucleic acid generated in accordance with the methods of the subject invention may be part of a transcriptional unit that may also contain 3′ and 5′ untranslated regions (UTRs) and a transcriptional terminator. The transcriptional unit may then be placed in an expression vector that can be used, e.g., in a method of transient or stable transfection of a host cell (e.g., a mammalian, yeast or melanophore cell). The expression vector can contain a selectable marker, e.g., neomycin resistance gene, to permit detection of those cells transfected with the subject GPR22 nucleic acid sequences (see, e.g., U.S. Pat. No. 4,704,362, the disclosure of which is herein incorporated by reference). Vectors, including single and dual expression cassette vectors are well known in the art (Ausubel, et al, Short Protocols in Molecular Biology, 3rd ed., Wiley & Sons, 1995; Sambrook, et al., Molecular Cloning: A Laboratory Manual, Second Edition, (1989) Cold Spring Harbor, N.Y.). Suitable expression vectors include viral vectors, plasmids and the like that are capable of being introduced into a host cell and of directing expression of the encoded GPR22 receptor polypeptide. Retroviral, adenoviral and adeno-associated viral vectors may also be used. GPR22 receptor polypeptide may be expressed in insect cells from substituted GPR22 nucleic acid generated in accordance with the methods of the subject invention using baculovirus (e.g., Pharmingen, San Diego Calif.). A variety of eukaryotic expression vectors are available to those in the art and include expression vectors which are commercially available (e.g., from Invitrogen, Carlsbad, Calif.; Clontech, Mountain View, Calif.; Stratagene, La Jolla, Calif.). Commercially available expression vectors include, by way of non-limiting example, CMV promoter-based vectors. One suitable expression vector is pCMV. The substituted GPR22 nucleic acid generated in accordance with the methods of the subject invention may also contain restriction sites, multiple cloning sites, primer-binding sites, ligatable ends, recombination sites etc., usually in order to facilitate the construction of a GPR22 expression cassette, which may then be introduced into a particular host cell.

Methods of introducing GPR22-encoding nucleic acids into cells are well known in the art. Suitable methods include electroporation, particle gun technology, calcium phosphate precipitation, direct microinjection, transfection, transduction, and the like. The choice of method is generally dependent on the type of cell being transformed and the circumstances under which the transformation is taking place (i.e., in vitro transformation). A general discussion of these methods can be found in Ausubel, et al, Short Protocols in Molecular Biology, 3rd ed., Wiley & Sons, 1995. In some embodiments, lipofectamine and calcium mediated gene transfer technologies are used.

Suitable host cells of the invention include any eukaryotic cell capable of expressing the GPR22 receptor polypeptide encoded by a substituted GPR22 nucleic acid generated in accordance with the methods of the subject invention. The eukaryotic cell can be an animal cell (e.g., an insect, mammal, fish, amphibian, bird or reptile cell), a plant cell (for example, a maize or Arabidopsis cell), or a fungal cell (for example, a yeast cell, a S. cerevisiae cell). Typically, an animal host cell line is used, non-limiting examples of which are as follows: monkey kidney cells (COS cells), monkey kidney CVI cells transformed by SV40 (COS-7, ATCC CRL 1651); human embryonic kidney cells (HEK-293 [“293”] cells, Graham et al. J. Gen Virol. 36:59 (1977)); HEK-293T [“293T”] cells; baby hamster kidney cells (BHK, ATCC CCL 10); Chinese hamster ovary-cells (CHO, Urlaub and Chasin, Proc. Natl. Acad. Sci. (USA) 77:4216, (1980); Syrian golden hamster cells MCB3901 (ATCC CRL-9595); mouse sertoli cells (TM4, Mather, Biol. Reprod. 23:243-251 (1980)); monkey kidney cells (CVI ATCC CCL 70); african green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); Spodoptera frugiperda insect Sf9 cells (ATCC CRL-1711); human liver cells (hep G2, HB 8065); mouse mammary tumor (MMT 060562, ATCC CCL 51); TR1 cells (Mather et al., Annals N.Y. Acad. Sci. 383:44-68 (1982)); NIH/3T3 cells (ATCC CRL-1658); and mouse L cells (ATCC CCL-1). In certain embodiments, the animal host cell is a mammalian host cell. In certain embodiments, melanophores are used. Melanophores are skin cells found in lower vertebrates, such as amphibians. Materials and methods relating to the use of melanophores will be followed according to the disclosures of U.S. Pat. No. 5,462,856 and U.S. Pat. No. 6,051,386; incorporated herein by reference in their entireties. In certain embodiments, cardiomyocytes are used. In certain embodiments, the cardiomyocytes are mammalian; in certain embodiments, the cardiomyocytes are neonatal rat ventricular myocytes (NRVM), which can be obtained by methods well known in the art [see, e.g., Adams et al., J Biol Chem (1996) 271:1179-1186; the disclosure of which is herein incorporated by reference in its entirety]. Additional cell lines will become apparent to those of ordinary skill in the art, and a wide variety of cell lines are available from the American Type Culture Collection, 10801 University Boulevard, Manassas, Va. 20110-2209.

Accordingly, in one embodiment, the invention is directed to a method for producing a recombinant host cell that involves transfecting a suitable host cell (e.g., a mammalian, yeast or melanophore cell) with an expression vector containing a substituted mammalian GPR22 nucleic acid providing for enhanced expression of the encoded mammalian GPR22 receptor polypeptide, as described above, to produce a transfected host cell and culturing the host cell under conditions sufficient to express the mammalian GPR22 receptor from the expression vector. As will be described in more detail below, a host cell transfected in this way may be used in methods for showing that a substituted mammalian GPR22-encoding nucleic acid generated in accordance with the methods described herein is an expression-enhanced nucleic acid. As will be described in more detail below, a host cell transfected in this way may be used in methods for identifying compounds that modulate mammalian GPR22 receptors. Generally, these methods involve contacting the candidate compound with a recombinant host cell that has been transfected with an expression vector comprising a substituted GPR22 nucleic acid providing for enhanced expression of the encoded GPR22 receptor polypeptide and measuring the ability of the compound to inhibit or stimulate functionality of the expressed GPR22 receptor, wherein inhibition or stimulation of functionality is indicative of the candidate compound being a modulator of the GPR22 receptor (non-limiting examples of which include agonist, partial agonist, inverse agonist, and antagonist). In certain embodiments, the method for producing a mammalian GPR22 receptor polypeptide involves transiently transfecting the suitable host cell. In certain embodiments, the method for producing a mammalian GPR22 receptor polypeptide involves stably transfecting the suitable host cell.

Additionally, in one embodiment, the invention is directed to a non-human mammal transgenic for a human GPR22 receptor, wherein the GPR22 receptor is encoded by a substituted nucleic acid generated in accordance with the methods of the subject invention. In another embodiment, a method of using a transgenic non-human mammal to identify whether a candidate compound has efficacy for preventing or treating a disease or disorder related to a mammalian GPR22 receptor is provided. In some embodiments, the disease or disorder related to the mammalian GPR22 receptor is myocardial ischemia or a condition related thereto, including but not limited to myocardial infarction and congestive heart failure. In some embodiments, the disease or disorder related to the mammalian GPR22 receptor is cerebral ischemia or a condition related thereto, including but not limited to ischemic stroke. In some embodiments, the disease or disorder related to the mammalian GPR22 receptor is Alzheimer's disease. In another embodiment, a method of using a transgenic non-human mammal to identify whether a candidate compound has efficacy for cardioprotection in a mammal is provided. In another embodiment, a method of using a transgenic non-human mammal to identify whether a candidate compound has efficacy for neuroprotection in a mammal is provided.

Assessing Expression Levels of a GPR22—Encoding Nucleic Acid

Once GPR22-encoding nucleic acid is introduced into a suitable host cell (e.g., a mammalian or a melanophore host cell) for transient or stable expression, expression level of the encoded GPR22 polypeptide may be assessed by any method well known in the art. For instance, provision of enhanced expression by a non-endogenous substituted GPR22 nucleic acid generated in accordance with the methods of the subject invention from a first nucleic acid can be shown by comparing for a fixed amount of transfected DNA the expression of the GPR22 receptor polypeptide encoded by the first nucleic acid or by a wild-type GPR22 nucleic acid with the expression of the GPR22 receptor polypeptide encoded by the substituted GPR22 nucleic acid. In some embodiments, the wild-type GPR22 nucleic acid is wild-type human GPR22 nucleic acid. In some embodiments, the wild-type GPR22 nucleic acid is wild-type human GPR22 nucleic acid having SEQ ID NO: 1 or SEQ ID NO: 5. Said comparing may be by any suitable method known in the art. In some embodiments, said comparing is by a process comprising the measurement of a level of a second messenger, non-limiting examples of which include cyclic AMP (cAMP), cyclic GMP (cGMP), inositol triphosphate (IP₃), diacylglycerol (DAG), Ca²⁺, and MAP kinase activity.

By way of illustration and not limitation, expression levels can be determined, by measuring GPR22 mRNA levels, particularly steady-state mRNA levels (see, e.g. Example 13), by assessing translation of the GPR22 mRNA, or by assessing expression of the GPR22 polypeptide (e.g., measuring a level of steady-state GPR22 polypeptide expression, see Example 12). It is expressly contemplated that antibodies that recognize GPR22 receptor polypeptide or that recognize an epitope tag fused to GPR22 receptor polypeptide may be used in methods of measuring a level of steady-state GPR22 polypeptide expression (exemplary commercially available antibodies are provided infra). In certain embodiments, the level of steady-state GPR22 polypeptide expression is a level of cell surface expression of the GPR22 polypeptide. Methods well known in the art for using an antibody to assess a level of steady-state polypeptide expression include but are not limited to immunostaining of fixed cells, flow cytometry, radioimmunoassay, cell-based ELISA, and quantitative Western blot. Additionally, as set forth further below and in the Examples section, GPR22 polypeptide expression may be assessed by a process comprising measuring GPR22 functionality, e.g., by a process comprising measuring a level of intracellular IP₃, measuring a level of intracellular cAMP, measuring a level of intracellular Ca²⁺, or the like. (See, e.g., Examples 10 and 11.) Exemplary functional assays are described below in more detail.

Assessing GPR22 Expression Using Second Messenger-Based Assays

GPR22 is a Gi coupled receptor exhibiting detectable constitutive activity.

a. cAMP Assay

Gi inhibits the enzyme adenylyl cyclase. Adenylyl cyclase catalyzes the conversion of ATP to cAMP; thus, the activated GPR22 receptor that is coupled to the Gi protein is associated with decreased cellular levels of cAMP. [See, generally, “Indirect Mechanisms of Synaptic Transmission,” Chapter 8, From Neuron to Brain (3^{rd} Edition) Nichols J. G. et al, Editors, Sinauer Associates, Inc. (1992).] Accordingly, assays that detect cAMP can be utilized to assess GPR22 expression by measuring a level of GPR22 constitutive activity (i.e., wherein levels of intracellular cAMP are constitutively decreased). A variety of approaches known in the art for measuring cAMP can be utilized, for instance, in some embodiments one approach relies upon the use of anti-cAMP antibodies in an ELISA-based format.

For an activated receptor such as GPR22 that is coupled to Gi so as to inhibit the formation of cAMP, however, assays measuring cAMP levels can be challenging because the variable being measured is a signal decrease upon activation. Accordingly, in some embodiments, an effective technique in measuring the decrease in production of cAMP as an indication of activation of a GPR22 receptor that couples to Gi upon activation can be accomplished by co-transfecting a signal enhancer, e.g., a non-endogenous, constitutively activated receptor that couples to Gs upon activation (or an endogenous Gs coupled receptor for which an agonist is known).

Activation of a Gs coupled receptor leads to an increase in production of cAMP. Activation of a Gi coupled receptor leads to a decrease in production of cAMP. Thus, the co-transfection approach is intended to advantageously exploit these “opposite” affects. For example, co-transfection of a non-endogenous, constitutively activated Gs coupled receptor (the “signal enhancer”) with empty expression vector provides a baseline cAMP signal (i.e., although the Gi coupled receptor will decrease cAMP levels, this “decrease” will be relative to the substantial increase in cAMP levels established by the constitutively activated Gs coupled signal enhancer). By co-transfecting the signal enhancer with the GPR22 receptor, an inverse agonist of the Gi coupled receptor will increase the measured cAMP signal, while an agonist of the Gi coupled receptor (or a constitutively active Gi coupled receptor in the absence of agonist) will decrease this signal. Once enhanced expression of the GPR22 polypeptide receptor encoded by a substituted GPR22 nucleic acid generated in accordance with the methods of the subject invention has been identified in this way, this system may then be used to identify candidate compounds that modulate the GPR22 receptor.

In certain embodiments, a non-endogenous substituted GPR22 nucleic acid is an expression-enhanced GPR22 nucleic acid if in the foregoing assay comprising or not comprising a signal enhancer the suppression or inhibition or decrease of the level of intracellular cAMP accumulation by the substituted GPR22 nucleic acid is at least about 1.5 times, at least about 2.0 times, at least about 2.5 times, at least about 3.0 times, at least about 3.5 times, at least about 4.0 times, at least about 4.5 times, or at least about 5.0 times that by a wild-type GPR22 nucleic acid. In some embodiments, the wild-type GPR22 nucleic acid is wild-type human GPR22 nucleic acid. In some embodiments, the wild-type GPR22 nucleic acid is wild-type human GPR22 nucleic acid having SEQ ID NO: 1 or SEQ ID NO: 5. (See, e.g., Example 10, infra.)

b. IP₃ Assay

Additionally, a different co-transfection approach involves converting Gi signaling to Gq signaling by co-transfecting a host cell with a Gq(del)/Gi chimeric G protein that acts to convert the Gi signaling of GPR22 receptor polypeptide to Gq signaling. Gq is associated with activation of the enzyme phospholipase C, which in turn hydrolyzes the phospholipid PIP₂ resulting in the release of two intracellular messengers: diacyclglycerol (DAG) and inositol 1,4,5-triphosphate (IP₃). Activation of a Gq coupled receptor is associated with increased accumulation of intracellular IP₃ and an increase in the level of intracellular Ca²⁺. [See generally, “Indirect Mechanisms of Synaptic Transmission,” Chapter 8, From Neuron to Brain (3^{rd} Edition) Nichols J. G. et al, Editors, Sinauer Associates, Inc. (1992).] Hence, Gq(del)/Gi chimeric G protein converts GPR22 receptor Gi signaling to Gq signaling such that the second messenger inositol triphosphate (IP₃) or diacylglycerol (DAG) or Ca²⁺, e.g., can be measured in lieu of cAMP production.

Accordingly, as GPR22 receptor is exhibits a detectable level of constitutive activity, this assay can measure GPR22 receptor expression by measuring a level of intracellular IP₃ accumulation or a level of intracellular Ca²⁺. By way of illustration and not limitation, a greater level of intracellular IP₃ accumulation is associated with a greater level of GPR22 receptor expression. This approach may be used to show that a non-endogenous substituted GPR22 nucleic acid generated in accordance with the methods of the subject invention is an expression-enhanced GPR22 nucleic acid (see, e.g., Example 11, infra).

Once enhanced expression of the GPR22 polypeptide receptor encoded by a substituted GPR22 nucleic acid generated in accordance with the methods of the subject invention has been identified in this way, this system may then be used to identify candidate compounds that modulate the GPR22 receptor. For example, an agonist to a Gq(del)/G1-coupled GPR22 receptor will increase a level of intracellular IP₃ accumulation or a level of intracellular Ca²⁺, whereas an inverse agonist or an antagonist will decrease a level of intracellular IP₃ accumulation or a level of intracellular Ca²⁺. By way of illustration and not limitation, a level of intracellular Ca²⁺ can be measured by fluorometric imaging plate reader (FLIPR) assay, as described infra.

In certain embodiments, a non-endogenous substituted GPR22 nucleic acid is an expression-enhanced GPR22 nucleic acid if in the foregoing assay comprising co-transfection with Gq(del)/Gi chimeric G protein the level of stimulation or increase of IP₃ accumulation by GPR22 encoded by the substituted nucleic acid is at least about 130%, at least about 150%, at least about 200%, at least about 250%, at least about 300%, at least about 350%, at least about 400%, at least about 450%, at least about 500%, at least about 550%, at least about 600%, at least about 650%, at least about 700%, at least about 750%, at least about 800%, at least about 850%, at least about 900%, at least about 950%, or at least about 1000% the level of stimulation of IP₃ accumulation by GPR22 encoded by a wild-type nucleic acid. In some embodiments, the wild-type GPR22 nucleic acid is wild-type human GPR22 nucleic acid. In some embodiments, the wild-type GPR22 nucleic acid is wild-type human GPR22 nucleic acid having SEQ ID NO: 1 or SEQ ID NO: 5. (See, e.g., Example 11, infra.)

Modulator Assays Using Synthetic Gpr22—Encoding Nucleic Acids of the Invention

In one embodiment, the polynucleotide expression constructs, vectors, and host cells comprising a substituted GPR22 nucleic acid generated in accordance with the methods of the subject invention are used in assays to screen candidate compounds as modulators of GPR22 receptor polypeptide. Agents that modulate (e.g., increase or decrease) GPR22 receptor activity may be identified by contacting a candidate compound with a recombinant host cell expressing a GPR22 polypeptide encoded by a nucleic acid generated in accordance with the methods of the present invention.

Accordingly, the invention provides methods of screening test compounds to identify ligands of a GPR22 polypeptide. Although several different assays are set forth herein below, these are for illustrative purposes only and should not be construed as limiting the subject invention in any way. In many embodiments, these methods are in vitro methods, involving contacting a cell producing an expression-enhanced GPR22 receptor polypeptide with a test compound, and determining the effect of the test compound on an observable intracellular activity (e.g., cAMP or IP₃ or Ca²⁺ accumulation) in relation to a suitable control. In many embodiments, a suitable control is the expression-enhanced GPR22 receptor polypeptide in the absence of the test compound. A GPR22 receptor modulator usually increases or decreases the amount of a detectable agent (i.e., cAMP or IP₃ or Ca²⁺) in comparison to controls. For instance, where the detectable agent is an intracellular second messenger (i.e., cAMP or IP₃ or Ca²⁺) a GPR22 receptor modulator will either increase or decrease the intracellular accumulation of that second messenger. (See below Examples 10 and 11.) In certain embodiments, a test compound identified as a GPR22 receptor modulator decreases the amount of the detectable agent by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or by at least about 99%, as compared to controls. In certain embodiments, a test compound identified as a GPR22 receptor modulator increases the amount of the detectable agent by at least about 10%, at least about 20%, at least about 40%, at least about 60%, at least about 80%, at least about 100%, at least about 150%, at least about 200%, at least about 300%, at least about 500%, at least about 10-fold or by at least about 20-fold, or more, as compared to controls.

In an exemplary embodiment, an expression-enhanced GPR22 nucleic acid generated in accordance with the methods of the subject invention is introduced into a suitable host cell, and the cell is incubated under conditions that provide for expression of the encoded GPR22 polypeptide. The amount of a detectable agent (e.g., cAMP or IP₃ or Ca²⁺) is determined for a cell producing the expression-enhanced GPR22 polypeptide, or group of such cells, in the presence and in the absence of the test compound. In certain embodiments, the amount of the detectable agent (e.g., cAMP or IP₃ or Ca²⁺) in a cell producing the expression-enhanced GPR22 polypeptide is determined prior to its contact with a test compound, and also determined after the cell has been contacted with the test compound, usually at least about 10 minutes, at least about 30 minutes, at least about 1 hr, at least about 2 hr, at least about 4 hr, at least about 8 hr, at least about 12 hr or at least about 24 hr or more after the candidate test compound is contacted. In certain embodiments, said determinations are made in parallel rather than in series. Detection of the detectable agent for a cell producing the expression-enhanced GPR22 receptor polypeptide, as described above, may be done by any suitable method.

A variety of different test compounds may be screened by the above methods. Test compounds encompass numerous chemical classes, though typically they are organic molecules, preferably small organic compounds having a molecular weight of more than 50 and less than about 2,500 daltons. Test compounds comprise functional groups necessary for structural interaction with proteins, particularly hydrogen bonding, and typically include at least an amine, carbonyl, hydroxyl or carboxyl group, preferably at least two of the functional chemical groups. The test compounds often comprise cyclical carbon or heterocyclic structures and/or aromatic or polyaromatic structures substituted with one or more of the above functional groups. Test compounds are also found among biomolecules including peptides, saccharides, fatty acids, steroids, purines, pyrimidines, derivatives, structural analogs or combinations thereof.

Test compounds may be obtained from a wide variety of sources including libraries of synthetic or natural compounds. Candidate compounds can be tested by screening chemical libraries for molecules that modulate, e.g., inhibit, antagonize, or agonize the activity of a GPR22 receptor. Chemical libraries can be peptide libraries, peptidomimetic libraries, chemically synthesized libraries, recombinant, e.g., phage display libraries, in vitro translation-based libraries, and other non-peptide synthetic organic libraries, such as libraries of endogenous compounds known to have biological activity, etc. Endogenous candidate compounds comprising biological materials, such as but not limited to plasma and tissue extracts can also be screened.

In some embodiments direct identification of candidate compounds is conducted in conjunction with compounds generated via combinatorial chemistry techniques, whereby thousands of compounds are randomly prepared for such analysis. The candidate compound may be a member of a chemical library that may comprise any convenient number of individual members (e.g., tens to hundreds to thousand to millions of suitable compounds) for example peptides, peptoids, other oligomeric compounds (cyclic or linear), and template-based smaller molecules, for example low molecular weight and potential therapeutic agents (e.g., benzodiazepines, hydantoins, biaryls, carbocyclic and polycyclic compounds, naphthalenes, phenothiazines, acridines, steroids, carbohydrate and amino acid derivatives, dihydropyridines, benzhydryls and heterocycles, trizines, indoles, thiazolidines etc,). The types of compounds listed are illustrative, but not limiting.

Exemplary chemical libraries are commercially available from several sources (ArQule, Tripos/PanLabs, ChemDesign, Pharmacopoeia). In some cases, these chemical libraries are generated using combinatorial strategies that encode the identity of each member of the library on a substrate to which the member compound is attached, thus allowing direct and immediate identification of a molecule that is an effective modulator. Thus, in many combinatorial approaches, the position on a plate of a compound specifies that compound's composition. By such methods, many candidate molecules can be screened.

### Pharmaceutical Compositions

Candidate compounds selected for further development (including a candidate compound identified as a modulator or as a ligand of a mammalian GPR22 receptor in accordance with the methods of the subject invention) can be formulated into pharmaceutical compositions using techniques well known to those in the art. Suitable pharmaceutically acceptable carriers are available to those in the art; for example, see Remington's Pharmaceutical Sciences, 16^{th} edition, 1980, Mack Publishing Co. (Osol et al., eds.).

### Kits

Also provided are kits comprising reagents that find use in practicing the subject methods, as described above. For example, in some embodiments, kits for identifying GPR22 modulators are provided that include a composition comprising an expression-enhanced GPR22 nucleic acid generated in accordance with the methods of the subject invention that is operatively linked to a suitable host cell promoter. In another embodiment, the expression enhanced GPR22 nucleic acid may form part of a transcriptional unit that includes one or more of the following: 3′ and 5′ untranslated regions (UTRs) and a transcriptional terminator. In a further embodiment, the expression-enhanced GPR22 nucleic acid may further be placed in an expression vector that can provide for expression of the encoded GPR22 receptor polypeptide in a host cell.

The kit components may be present in a storage container and/or a container that is employed during the practicing of the assay for which the kit was designed. In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

The practice of the present invention will employ, unless otherwise indicated, conventional techniques of cell biology, cell culture, molecular biology (including PCR), vaccinology, microbiology, recombinant DNA, and immunology, which are within the skill of the art. Such techniques are explained fully in the literature. See, for example, Molecular Cloning A Laboratory Manual, 2nd Ed., Sambrook et al., ed., Cold Spring Harbor Laboratory Press: (1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); and in Ausubel et al., Current Protocols in Molecular Biology, John Wiley and Sons, Baltimore, Md. (1989).

### Example 1

### Receptor Expression

Although a variety of cells are available to the art for the expression of proteins, it is preferred that eukaryotic cells be utilized and more preferred that animal cells (e.g., mammalian cells or melanophore cells) be utilized. The primary reason for this is predicated upon practicalities, i.e., utilization of, e.g., yeast cells for the expression of a GPCR, while possible, introduces into the protocol a non-animal cell which may not (indeed, in the case of yeast, does not) include the receptor-coupling, genetic-mechanism and secretary pathways that have evolved for, e.g., mammalian systems-thus, results obtained in non-animal cells, while of potential use, are not as preferred as those obtained in, e.g., mammalian cells or melanophore cells. Of the mammalian cells, CHO, COS-7, MCB3901, 293 and 293T cells are particularly preferred, although the specific mammalian cell utilized can be predicated upon the particular needs of the artisan. In some embodiments, cardiomyocytes obtained from a mammal may be used. See infra as relates to melanophores.

### a. Transient Transfection

On day one, 4×10⁶/10 cm dish of 293 cells are plated out. On day two, two reaction tubes are prepared (the proportions to follow for each tube are per plate): tube A is prepared by mixing 4 μg DNA (e.g., pCMV vector; pCMV vector with GPR22 (where it is understood that “GPR22” may be wild-type GPR22 nucleic acid or expression-enhanced GPR22 nucleic acid generated in accordance with the methods of the subject invention or other mammalian GPR22-encoding nucleic acid), etc.) in 0.5 ml serum free DMEM (Gibco BRL); tube B is prepared by mixing 24 μl lipofectamine (Gibco BRL) in 0.5 ml serum free DMEM. Tubes A and B are admixed by inversions (several times), followed by incubation at room temperature for 30-45 min. The admixture is referred to as the “transfection mixture”. Plated 293 cells are washed with 1×PBS, followed by addition of 5 ml serum free DMEM. 1 ml of the transfection mixture is added to the cells, followed by incubation for 4 hrs at 37° C./5% CO₂. The transfection mixture is removed by aspiration, followed by the addition of 10 ml of DMEM/10% Fetal Bovine Serum. Cells are incubated at 37° C./5% CO₂. After 48 hr incubation, cells are harvested and utilized for analysis.

### b. Stable Cell Lines

Approximately 12×10⁶ 293 cells are plated on a 15 cm tissue culture plate and grown in DME High Glucose Medium containing ten percent fetal bovine serum and one percent sodium pyruvate, L-glutamine, and antibiotics. Twenty-four hours following plating of 293 cells (or to 80% confluency), the cells are transfected using 12 μg of DNA (e.g., pCMV-neo^{r} vector with GPR22 (where it is understood that “GPR22” may be wild-type GPR22 nucleic acid or expression-enhanced GPR22 nucleic acid generated in accordance with the methods of the subject invention or other mammalian GPR22-encoding nucleic acid). The 12 μg of DNA is combined with 60 μl of lipofectamine and 2 ml of DME High Glucose Medium without serum. The medium is aspirated from the plates and the cells are washed once with medium without serum. The DNA, lipofectamine, and medium mixture are added to the plate along with 10 mL of medium without serum. Following incubation at 37° C. for four to five hours, the medium is aspirated and 25 ml of medium containing serum is added. Twenty-four hours following transfection, the medium is aspirated again, and fresh medium with serum is added. Forty-eight hours following transfection, the medium is aspirated and medium with serum is added containing geneticin (G418 drug) at a final concentration of 500 μg/ml. The transfected cells now undergo selection for positively transfected cells containing the G418 resistance gene. The medium is replaced every four to five days as selection occurs. During selection, cells are grown to create stable pools, or split for stable clonal selection.

### Example 2

### Assays for Determination of GPCR Activation (e.g., Screening Assays)

A variety of approaches are available for assessment of activation of GPCRs, such as for screening assays. The following are illustrative; those of ordinary skill in the art are credited with the ability to determine those techniques that are preferentially beneficial for the needs of the artisan.

### a. Membrane Binding Assays: [³⁵S]GTPγS Assay

When a G protein-coupled receptor is in its active state, either as a result of ligand binding or constitutive activation, the receptor couples to a G protein and stimulates the release of GDP and subsequent binding of GTP to the G protein. The alpha subunit of the G protein-receptor complex acts as a GTPase and slowly hydrolyzes the GTP to GDP, at which point the receptor normally is deactivated. Activated receptors continue to exchange GDP for GTP. The non-hydrolyzable GTP analog, [³⁵S]GTPγS, can be utilized to demonstrate enhanced binding of [³⁵S]GTPγS to membranes expressing activated receptors. The advantage of using [³⁵S]GTPγS binding to measure activation is that: (a) it is generically applicable to all G protein-coupled receptors; (b) it is proximal at the membrane surface making it less likely to pick-up molecules which affect the intracellular cascade.

The assay utilizes the ability of G protein coupled receptors to stimulate [³⁵S]GTPγS binding to membranes expressing the relevant receptors. The assay can, therefore, be used to screen candidate compounds as modulators of GPR22 receptor, e.g. an expression-enhanced GPR22 receptor produced in accordance with the methods of the subject invention. The assay is generic and has application to drug discovery at all G protein-coupled receptors. The [³⁵S]GTPγS assay is incubated in 20 mM HEPES and between 1 and about 20 mM MgCl₂ (this amount can be adjusted for optimization of results, although 20 mM is preferred) pH 7.4, binding buffer with between about 0.3 and about 1.2 nM [³⁵S]GTPγS (this amount can be adjusted for optimization of results, although 1.2 nM is preferred) and 12.5 to 75 μg membrane protein (e.g, 293 cells expressing GPR22 receptor, e.g. an expression-enhanced GPR22 receptor produced in accordance with the methods of the subject invention; this amount can be adjusted for optimization) and 10 μM GDP (this amount can be changed for optimization) for 1 hour. Wheat germ agglutinin beads (25 μl; Amersham) are then added and the mixture incubated for another 30 minutes at room temperature. The tubes are then centrifuged at 1500×g for 5 minutes at room temperature and then counted in a scintillation counter.

### b. Cell-Based cAMP Assay for Gi Coupled GPCRs

TSHR is a Gs coupled GPCR that causes the accumulation of cAMP upon activation. TSHR will be constitutively activated by mutating amino acid residue 623 (i.e., changing an alanine residue to an isoleucine residue). A Gi coupled receptor is expected to inhibit adenylyl cyclase, and, therefore, to decrease the level of cAMP production, which can make assessment of cAMP levels challenging. An effective technique for measuring the decrease in production of cAMP as an indication of activation of a Gi coupled receptor can be accomplished, e.g., by co-transfecting nucleic acid encoding the Gi coupled receptor with nucleic acid encoding non-endogenous, constitutively activated TSHR (TSHR-A623I) (or an endogenous, constitutively active Gs coupled receptor) as a “signal enhancer.” Transfection only of nucleic acid encoding the “signal enhancer” establishes a baseline level of cAMP. Accordingly, nucleic acid encoding GPR22, e.g. wild-type GPR22 nucleic acid or expression-enhanced GPR22 nucleic acid generated in accordance with the methods of the subject invention, is co-transfected with nucleic acid encoding the signal enhancer, and it is this material that can be used for assessing the level of GPR22 receptor expression or for screening. Such an approach can be utilized to effectively generate a signal when a cAMP assay is used. In some embodiments, this approach is preferably used for assessing a level of GPR22 receptor expression or in the identification of candidate compounds as modulators of GPR22 receptor. It is noted that for a Gi coupled GPCR such as GPR22, when this approach is used, an inverse agonist of the GPCR will increase the cAMP signal and an agonist will decrease the cAMP signal.

On day one, 4×10⁶ 293 cells per 10 cm plate are plated out. On day two, two reaction tubes are prepared (the proportions to follow for each tube are per plate): tube A is prepared by mixing 2 μg DNA of each receptor transfected into the mammalian cells, for a total of 4 μg DNA (e.g., pCMV vector; pCMV vector with mutated TSHR (TSHR-A623I); TSHR-A623I and GPR22, e.g. wild-type GPR22 nucleic acid or expression-enhanced GPR22 nucleic acid generated in accordance with the methods of the subject invention; etc.) in 0.5 ml serum free DMEM (Irvine Scientific, Irvine, Calif.); tube B is prepared by mixing 24 μl lipofectamine (Gibco BRL) in 0.5 ml serum free DMEM. Tubes A and B are then admixed by inversions (several times), followed by incubation at room temperature for 30-45 min. The admixture is referred to as the “transfection mixture”. Plated 293 cells are washed with 1×PBS, followed by addition of 5 ml serum free DMEM. 1.0 ml of the transfection mixture is then added to the cells, followed by incubation for 4 hrs at 37° C./5% CO₂. The transfection mixture is then removed by aspiration, followed by the addition of 10 ml of DMEM/10% Fetal Bovine Serum. Cells are then incubated at 37° C./5% CO₂. After 24 hr incubation, cells are then harvested and utilized for analysis.

A Flash Plate™ Adenylyl Cyclase kit (New England Nuclear; Cat. No. SMP004A) is designed for cell-based assays, but can be modified for use with crude plasma membranes depending on the need of the skilled artisan. The Flash Plate wells contain a scintillant coating which also contains a specific antibody recognizing cAMP. The cAMP generated in the wells can be quantitated by a direct competition for binding of radioactive cAMP tracer to the cAMP antibody. The following serves as a brief protocol for the measurement of changes in cAMP levels in whole cells that express GPR22, e.g. wild-type GPR22 nucleic acid or expression-enhanced GPR22 nucleic acid generated in accordance with the methods of the subject invention.

Transfected cells are harvested approximately twenty-four to forty-eight hours after transient transfection. Media is carefully aspirated off and discarded. 10 ml of PBS is gently added to each dish of cells followed by careful aspiration. 1 ml of Sigma cell dissociation buffer and 3 ml of PBS are added to each plate. Cells are pipetted off the plate and the cell suspension is collected into a 50 ml conical centrifuge tube. Cells are then centrifuged at room temperature at 1,100 rpm for 5 min. The cell pellet is carefully re-suspended into an appropriate volume of PBS (about 3 ml/plate). The cells are then counted using a hemocytometer and additional PBS is added to give the appropriate number of cells (with a final volume of about 50 μl/well). cAMP standards and Detection Buffer (comprising 1 μCi of tracer [125I] cAMP (50 μl) to 11 ml Detection Buffer) are prepared and maintained in accordance with the manufacturer's instructions. Assay Buffer should be prepared fresh for screening and contains 50 μl of Stimulation Buffer, 3 μl of test compound (12 μM final assay concentration) and 50 μl cells, Assay Buffer can be stored on ice until utilized. The assay can be initiated by addition of 50 μl of cAMP standards to appropriate wells followed by addition of 50 μl of PBS to wells H-11 and H12. Fifty μl of Stimulation Buffer is added to all wells. Selected compounds (e.g., TSH) are added to appropriate wells using a pin tool capable of dispensing 3 μl of compound solution, with a final assay concentration of 12 μM test compound and 100 μl total assay volume. The cells are then added to the wells and incubated for 60 min at room temperature. 100 μl of Detection Mix containing tracer cAMP is then added to the wells. Plates are then incubated additional 2 hours followed by counting in a Wallac MicroBeta scintillation counter. Values of cAMP/well are then extrapolated from a standard cAMP curve which is contained within each assay plate.

### c. Reporter-Based Assays

1. CRE-Luc Reporter Assay (Gs-Associated Receptors)

293 and 293T cells are plated-out on 96 well plates at a density of 2×10⁴ cells per well and are transfected using Lipofectamine Reagent (BRL) the following day according to manufacturer instructions. A DNA/lipid mixture is prepared for each 6-well transfection as follows: 260 ng of plasmid DNA in 100 μl of DMEM is gently mixed with 2 μl of lipid in 100 μl of DMEM (the 260 ng of plasmid DNA consists of 200 ng of a 8×CRE-Luc reporter plasmid, 50 ng of pCMV-GPR22 (pCMV containing GPR22 nucleic acid, e.g. wild-type GPR22 nucleic acid or expression-enhanced GPR22 nucleic acid generated in accordance with the methods of the subject invention) or pCMV alone, and 10 ng of a GPRS expression plasmid (GPRS in pcDNA3 (Invitrogen)). The 8×CRE-Luc reporter plasmid was prepared as follows: vector SRIF-β-gal was obtained by cloning the rat somatostatin promoter (−71/+51) at BglV-HindIII site in the pβgal-Basic Vector (Clontech). Eight (8) copies of cAMP response element were obtained by PCR from an adenovirus template AdpCF126CCRE8 [see, Suzuki et al., Hum Gene Ther (1996) 7:1883-1893; the disclosure of which is herein incorporated by reference in its entirety) and cloned into the SRIF-β-gal vector at the Kpn-BglV site, resulting in the 8×CRE-β-gal reporter vector. The 8×CRE-Luc reporter plasmid was generated by replacing the beta-galactosidase gene in the 8×CRE-β-gal reporter vector with the luciferase gene obtained from the pGL3-basic vector (Promega) at the HindIII-BamHI site. Following 30 min. incubation at room temperature, the DNA/lipid mixture is diluted with 400 μl of DMEM and 100 μl of the diluted mixture is added to each well. 100 μl of DMEM with 10% FCS are added to each well after a 4 hr incubation in a cell culture incubator. The following day the transfected cells are changed with 200 μl/well of DMEM with 10% FCS. Eight (8) hours later, the wells are changed to 100 μl/well of DMEM without phenol red, after one wash with PBS. Luciferase activity is measured the next day using the LucLite™ reporter gene assay kit (Packard) following manufacturer instructions and read on a 1450 MicroBeta™ scintillation and luminescence counter (Wallac).

2. AP1 Reporter Assay (Gq-Associated Receptors)

A method to detect Gq stimulation depends on the known property of Gq-dependent phospholipase C to cause the activation of genes containing AP1 elements in their promoter. A Pathdetect™ AP-1 cis-Reporting System (Stratagene, Catalogue # 219073) can be utilized following the protocol set forth above with respect to the CREB reporter assay, except that the components of the calcium phosphate precipitate are 410 ng pAP1-Luc, 80 ng pCMV-GPR22 expression plasmid (pCMV containing GPR22 nucleic acid, e.g. wild-type GPR22 nucleic acid or expression-enhanced GPR22 nucleic acid generated in accordance with the methods of the subject invention), and 20 ng CMV-SEAP (secreted alkaline phosphatase expression plasmid; alkaline phosphatase activity is measured in the media of transfected cells to control for variations in transfection efficiency between samples).

3. SRF-Luc Reporter Assay (Gq-Associated Receptors)

One method to detect Gq stimulation depends on the known property of Gq-dependent phospholipase C to cause the activation of genes containing serum response factors in their promoter. A Pathdetect™ SRF-Luc-Reporting System (Stratagene) can be utilized to assay for Gq coupled activity in, e.g., COS7 cells. Cells are transfected with the plasmid components of the system and the indicated expression plasmid encoding the GPR22 polypeptide using a Mammalian Transfection™ Kit (Stratagene, Catalogue #200285) according to the manufacturer's instructions. Briefly, 410 ng SRF-Luc, 80 ng pCMV-GPR22 (PCMV containing GPR22 nucleic acid, e.g. wild-type GPR22 nucleic acid or expression-enhanced GPR22 nucleic acid generated in accordance with the methods of the subject invention) expression plasmid and 20 ng CMV-SEAP are combined in a calcium phosphate precipitate as per the manufacturer's instructions. Half of the precipitate is equally distributed over 3 wells in a 96-well plate, kept on the cells in a serum free media for 24 hours. The last 5 hours the cells are incubated with, e.g. 1 μM, test compound. Cells are then lysed and assayed for luciferase activity using a Luclite™ Kit (Packard, Cat. # 6016911) and “Trilux 1450 Microbeta” liquid scintillation and luminescence counter (Wallac) as per the manufacturer's instructions. The data can be analyzed using GraphPad Prism™ 2.0a (GraphPad Software Inc.).

d. Intracellular IP₃ Accumulation Assay (Gq-Associated Receptors)

On day 1, cells comprising GPR22 receptor polypeptide produced in accordance with the methods of the subject invention (e.g., cells co-transfected with Gq(del)/Gi chimeric G protein and with wild-type GPR22 nucleic acid or with expression-enhanced GPR22 nucleic acid generated in accordance with the methods of the subject invention or with other mammalian GPR22-encoding nucleic acid) can be plated onto 24 well plates, usually 1×10⁵ cells/well (although his number can be optimized. On day 2 cells can be transfected by first mixing 0.25 μg DNA in 50 μl serum free DMEM/well and 2 μl lipofectamine in 50 μl serum free DMEM/well. The solutions are gently mixed and incubated for 15-30 min at room temperature. Cells are washed with 0.5 ml PBS and 400 μl of serum free media is mixed with the transfection media and added to the cells. The cells are then incubated for 3-4 hrs at 37° C./5% CO₂ and then the transfection media is removed and replaced with 1 ml/well of regular growth media. On day 3 the cells are labeled with ³H-myo-inositol. Briefly, the media is removed and the cells are washed with 0.5 ml PBS. Then 0.5 ml inositol-free/serum free media (GIBCO BRL) is added/well with 0.25 μCi of ³H-myo-inositol/well and the cells are incubated for 16-18 hrs o/n at 37° C./5% CO₂. On Day 4 the cells are washed with 0.5 ml PBS and 0.45 ml of assay medium is added containing inositol-free/serum free media 10 μM pargyline 10 mM lithium chloride or 0.4 ml of assay medium and optionally 50 μl of test compound. The cells are then incubated for 30 min at 37° C. The cells are then washed with 0.5 ml PBS and 200 μl of fresh/ice cold stop solution (1M KOH; 18 mM Na-borate; 3.8 mM EDTA) is added/well. The solution is kept on ice for 5-10 min or until cells were lysed and then neutralized by 200 μl of fresh/ice cold neutralization sol. (7.5% HCl). The lysate is then transferred into 1.5 ml eppendorf tubes and 1 ml of chloroform/methanol (1:2) is added/tube. The solution is vortexed for 15 sec and the upper phase is applied to a Biorad AG1-X8™ anion exchange resin (100-200 mesh). Firstly, the resin is washed with water at 1:1.25 W/V and 0.9 ml of upper phase is loaded onto the column. The column is washed with 10 mls of 5 mM myo-inositol and 10 ml of 5 mM Na-borate/60 mM Na-formate. The inositol tris phosphates are eluted into scintillation vials containing 10 ml of scintillation cocktail with 2 ml of 0.1 M formic acid/1 M ammonium formate. The columns are regenerated by washing with 10 ml of 0.1 M formic acid/3M ammonium formate and rinsed twice with dd H₂O and stored at 4° C. in water.

### Example 3

### [³⁵S]GTPγS Assay

1. Membrane Preparation

In some embodiments membranes comprising GPR22 receptor polypeptide produced in accordance with the methods of the subject invention (e.g., cells transfected with wild-type GPR22 nucleic acid or with expression-enhanced GPR22 nucleic acid generated in accordance with the methods of the subject invention or with other mammalian GPR22-encoding nucleic acid) and for use in the identification of candidate compounds as, e.g.,. inverse agonists, agonists, or antagonists, are preferably prepared as follows:

a. Materials

“Membrane Scrape Buffer” is comprised of 20 mM HEPES and 10 mM EDTA, pH 7.4; “Membrane Wash Buffer” is comprised of 20 mM HEPES and 0.1 mM EDTA, pH 7.4; “Binding Buffer” is comprised of 20 mM HEPES, 100 mM NaCl, and 10 mM MgCl₂, pH 7.4.

b. Procedure

All materials are kept on ice throughout the procedure. Firstly, the media is aspirated from a confluent monolayer of cells, followed by rinse with 10 ml cold PBS, followed by aspiration. Thereafter, 5 ml of Membrane Scrape Buffer is added to scrape cells; this will be followed by transfer of cellular extract into 50 ml centrifuge tubes (centrifuged at 20,000 rpm for 17 minutes at 4° C.). Thereafter, the supernatant is aspirated and the pellet is resuspended in 30 ml Membrane Wash Buffer followed by centrifuge at 20,000 rpm for 17 minutes at 4° C. The supernatant is then aspirated and the pellet resuspended in Binding Buffer. This is then homogenized using a Brinkman Polytron™ homogenizer (15-20 second bursts until the all material is in suspension). This is referred to herein as “Membrane Protein”.

2. Bradford Protein Assay

Following the homogenization, protein concentration of the membranes is determined using the Bradford Protein Assay (protein can be diluted to about 1.5 mg/ml, aliquoted and frozen (−80° C.) for later use; when frozen, protocol for use is as follows: on the day of the assay, frozen Membrane Protein is thawed at room temperature, followed by vortex and then homogenized with a Polytron at about 12×1,000 rpm for about 5-10 seconds; it is noted that for multiple preparations, the homogenizer should be thoroughly cleaned between homogenization of different preparations).

a. Materials

Binding Buffer (as per above); Bradford Dye Reagent; Bradford Protein Standard is utilized, following manufacturer instructions (Biorad, cat. no. 500-0006).

b. Procedure

Duplicate tubes are prepared, one including the membrane, and one as a control “blank”. Each contains 800 μl Binding Buffer. Thereafter, 10 μl of Bradford Protein Standard (1 mg/ml) is added to each tube, and 10 μl of membrane Protein is then added to just one tube (not the blank). Thereafter, 200 μl of Bradford Dye Reagent is added to each tube, followed by vortex of each. After five (5) minutes, the tubes are re-vortexed and the material therein are transferred to cuvettes. The cuvettes are then read using a CECIL 3041 spectrophotometer, at wavelength 595.

3. Identification Assay

a. Materials

GDP Buffer consists of 37.5 ml Binding Buffer and 2 mg GDP (Sigma, cat. no. G-7127), followed by a series of dilutions in Binding Buffer to obtain 0.2 μM GDP (final concentration of GDP in each well is 0.1 μM GDP); each well comprising a candidate compound, has a final volume of 200 μl consisting of 100 μl GDP Buffer (final concentration, 0.1 μM GDP), 50 μl Membrane Protein in Binding Buffer, and 50 μl [³⁵S]GTPγS (0.6 nM) in Binding Buffer (2.5 μl [³⁵S]GTPγS per 10 ml Binding Buffer).

b. Procedure

Candidate compounds are preferably screened using a 96-well plate format (these can be frozen at −80° C.). Membrane Protein (or membranes with expression vector excluding the Target GPCR, as control), is homogenized briefly until in suspension. Protein concentration will then be determined using the Bradford Protein Assay set forth above. Membrane Protein (and control) is then diluted to 0.25 mg/ml in Binding Buffer (final assay concentration, 12.5 μg/well). Thereafter, 100 μl GDP Buffer is added to each well of a Wallac Scintistrip™ (Wallac). A 5 ul pin-tool is then used to transfer 5 μl of a candidate compound into such well (i.e., 5 μl in total assay volume of 200 μl is a 1:40 ratio such that the final screening concentration of the candidate compound is 10 μM). Again, to avoid contamination, after each transfer step the pin tool should be rinsed in three reservoirs comprising water (1×), ethanol (1×) and water (2×)—excess liquid should be shaken from the tool after each rinse and dried with paper and kimwipes. Thereafter, 50 μl of Membrane Protein is added to each well (a control well comprising membranes without GPR22 receptor polypeptide is also utilized), and pre-incubated for 5-10 minutes at room temperature. Thereafter, 50 μl of [³⁵S]GTPγS (0.6 nM) in Binding Buffer is added to each well, followed by incubation on a shaker for 60 minutes at room temperature (again, in this example, plates were covered with foil). The assay is then stopped by spinning of the plates at 4000 RPM for 15 minutes at 22° C. The plates will then be aspirated with an 8 channel manifold and sealed with plate covers. The plates will then be read on a Wallac 1450 using setting “Prot. #37” (as per manufacturer's instructions).

### Example 4

### Cyclic AMP Assay

Another assay approach for identifying candidate compounds as, e.g., inverse agonists, agonists, or antagonists, is accomplished by utilizing a cyclase-based assay. In addition to so identifying candidate compounds, this assay approach can be utilized as an independent approach to provide confirmation of the results from the [³⁵S]GTPγS approach as set forth in Example 3, supra.

A modified Flash Plate™ Adenylyl Cyclase kit (New England Nuclear; Cat. No. SMP004A) is preferably utilized for identification of candidate compounds as modulators of GPR22, preferably GPR22 produced in accordance with methods of the subject invention (e.g., cells transfected with wild-type GPR22 nucleic acid or with expression-enhanced GPR22 nucleic acid generated in accordance with the methods of the subject invention or with other mammalian GPR22-encoding nucleic acid), in accordance with the following protocol.

Transfected cells are harvested approximately three days after transfection. Membranes are prepared by homogenization of suspended cells in buffer containing 20 mM HEPES, pH 7.4 and 10 mM MgCl₂. Homogenization is performed on ice using a Brinkman Polytron™ for approximately 10 seconds. The resulting homogenate is centrifuged at 49,000×g for 15 minutes at 4° C. The resulting pellet is then resuspended in buffer containing 20 mM HEPES, pH 7.4 and 0.1 mM EDTA, homogenized for 10 seconds, followed by centrifugation at 49,000×g for 15 minutes at 4° C. The resulting pellet is then stored at −80° C. until utilized. On the day of direct identification screening, the membrane pellet is slowly thawed at room temperature, resuspended in buffer containing 20 mM HEPES, pH 7.4 and 10 mM MgCl₂, to yield a final protein concentration of 0.60 mg/ml (the resuspended membranes are placed on ice until use). cAMP standards and Detection Buffer (comprising 2 μCi of tracer {[¹²⁵I]cAMP (100 μl) to 11 ml Detection Buffer] are prepared and maintained in accordance with the manufacturer's instructions. Assay Buffer is prepared fresh for screening and contained 20 mM HEPES, pH 7.4, 10 mM MgCl₂, 20 mM phosphocreatine (Sigma), 0.1 units/ml creatine phosphokinase (Sigma), 50 μM GTP (Sigma), and 0.2 mM ATP (Sigma); Assay Buffer is then stored on ice until utilized.

Candidate compounds are added, preferably, to e.g. 96-well plate wells (3 μl/well; 12 μM final assay concentration), together with 40 μl Membrane Protein (30 μg/well) and 50 μl of Assay Buffer. This admixture is then incubated for 30 minutes at room temperature, with gentle shaking.

Following the incubation, 100 μl of Detection Buffer is added to each well, followed by incubation for 2-24 hours. Plates are then counted in a Wallac MicroBeta™ plate reader using “Prot. #31” (as per manufacturer's instructions).

### Example 5

### Gq(del)/Gi Fusion Construct

A Gq(del)/Gi fusion construct is a chimeric G protein whereby the first six (6) amino acids of the Gq-protein α-subunit (“Gαq”) are deleted and the last five (5) amino acids at the C-terminal end of Gαq are replaced with the corresponding amino acids of the Gαi subunit. Gq(del)/Gi chimeric G protein converts Gi signaling to Gq signaling such that the second messenger inositol triphosphate (IP₃) or diacylglycerol (DAG) or Ca²⁺, e.g., can be measured in lieu of cAMP production.

The Gq(del)/Gi fusion construct was designed as follows: the N-terminal six (6) amino acids (amino acids 2 through 7, having the sequence of TLESIM (SEQ ID NO: 15) of the Gαq-subunit were deleted and the C-terminal five (5) amino acids, having the sequence EYNLV (SEQ ID NO: 16) were replaced with the corresponding amino acids of the Gαi Protein, having the sequence DCGLF (SEQ ID NO: 17). This fusion construct was obtained by PCR using the following primers: 5′-gatcaagcttcCATGGCGTGCTGCCTGAGCGAGGAG-3′ (SEQ ID NO: 18) and 5′-gatcggatccTTAGAACAGGCCGCAGTCCTTCAGGTTCAGCTGCAGGATGGTG-3′ (SEQ ID NO: 19) and Plasmid 63313 (ATCC® Number 63313) which contains the mouse Goq-wild-type version with a hemagglutinin tag as a template. Nucleotides in lower case include cloning sites for HindIII/BamHI and spacers.

TaqPlus Precision DNA polymerase (Stratagene) was utilized for the amplification by the following cycles, with steps 2 through 4 repeated 35 times: 95° C. for 2 min; 95° C. for 20 sec; 56° C. for 20 sec; 72° C. for 2 min; and 72° C. for 7 min. The PCR product was cloned into a pCRII-TOPO vector (Invitrogen) and sequenced using the ABI Big Dye Terminator kit (P. E. Biosystems). Inserts from a TOPO clone containing the sequence of the fusion construct was shuttled into the expression vector pcDNA3.1(+) at the HindIII/BamHI site by a 2 step cloning process. See, SEQ ID NO: 20 for the nucleic acid sequence and SEQ ID NO: 21 for the encoded amino acid sequence of Gq(del)/Gi fusion construct.

### Example 6

### Fluorometric Imaging Plate Reader (FLIPR) Assay for the Measurement of Intracellular Calcium Concentration

Cells stably co-transfected with either pCMV-GPR22 (pCMV containing GPR22 nucleic acid, e.g. wild-type GPR22 nucleic acid or expression-enhanced GPR22 nucleic acid generated in accordance with the methods of the subject invention or other mammalian GPR22-encoding nucleic acid; experimental) or pCMV (negative control) and Gq(del)/Gi chimeric G protein from respective clonal lines are seeded into poly-D-lysine pretreated 96-well plates (Becton-Dickinson, #356640) at 5.5×10⁴ cells/well with complete culture medium (DMEM with 10% FBS, 2 mM L-glutamine, 1 mM sodium pyruvate) for assay the next day. To prepare Fluo4-AM (Molecular Probe, #F14202) incubation buffer stock, 1 mg Fluo4-AM is dissolved in 467 μl DMSO and 467 μl Pluoronic acid (Molecular Probe, #P3000) to give a 1 mM stock solution that can be stored at −20° C. for a month. Fluo4-AM is a fluorescent calcium indicator dye.

Candidate compounds are prepared in wash buffer (1×HBSS/2.5 mM Probenicid/20 mM HEPES at pH 7.4).

At the time of assay, culture medium is removed from the wells and the cells are loaded with 100 μl of 4 μM Fluo4-AM/2.5 mM Probenicid (Sigma, #P8761)/20 mM HEPES/complete medium at pH 7.4. Incubation at 37° C./5% CO₂ is allowed to proceed for 60 min.

After the 1 hr incubation, the Fluo4-AM incubation buffer is removed and the cells are washed 2× with 100 μl wash buffer. In each well is left 100 μl wash buffer. The plate is returned to the incubator at 37° C./5% CO₂ for 60 min.

FLIPR (Fluorometric Imaging Plate Reader; Molecular Device) is programmed to add 50 μl candidate compound on the 30th second and to record transient changes in intracellular calcium concentration ([Ca²⁺]) evoked by the candidate compound for another 150 seconds. Total fluorescence change counts are used to determine agonist activity using the FLIPR software. The instrument software normalizes the fluorescent reading to give equivalent initial readings at zero.

In some embodiments, cells are stably co-transfected with either pCMV-GPR22 (pCMV containing GPR22 nucleic acid, e.g. wild-type GPR22 nucleic acid or expression-enhanced GPR22 nucleic acid generated in accordance with the methods of the subject invention or other mammalian GPR22-encoding nucleic acid; experimental) or pCMV (negative control) and either Gα15 or Gα16 promiscuous G protein.

Although the foregoing provides a FLIPR assay for agonist activity using stably transfected cells, a person of ordinary skill in the art would readily be able to modify the assay in order to characterize antagonist activity. The person of ordinary skill in the art would also readily appreciate that, alternatively, transiently transfected cells could be used.

### Example 7

### MAP Kinase Assay

MAP kinase (mitogen activated kinase) may be monitored to evaluate receptor activation. MAP kinase can be detected by several approaches. One approach is based on an evaluation of the phosphorylation state, either unphosphorylated (inactive) or phosphorylated (active). The phosphorylated protein has a slower mobility in SDS-PAGE and can therefore be compared with the unstimulated protein using Western blotting. Alternatively, antibodies specific for the phosphorylated protein are available (New England Biolabs) which can be used to detect an increase in the phosphorylated kinase. In either method, cells are stimulated with the test compound and then extracted with Laemmli buffer. The soluble fraction is applied to an SDS-PAGE gel and proteins are transferred electrophoretically to nitrocellulose or Immobilin. Immunoreactive bands are detected by standard Western blotting technique. Visible or chemiluminescent signals are recorded on film and may be quantified by densitometry.

Another approach is based on evaluation of the MAP kinase activity via a phosphorylation assay. Cells are stimulated with the test compound and a soluble extract is prepared. The extract is incubated at 30° C. for 10 min with gamma-³²P-ATP, an ATP regenerating system, and a specific substrate for MAP kinase such as phosphorylated heat and acid stable protein regulated by insulin, or PHAS-I. The reaction is terminated by the addition of H₃PO₄ and samples are transferred to ice. An aliquot is spotted onto Whatman P81 chromatography paper, which retains the phosphorylated protein. The chromatography paper is washed and counted for ³²P is a liquid scintillation counter. Alternatively, the cell extract is incubated with gamma-³²P-ATP, an ATP regenerating system, and biotinylated myelin basic protein bound by streptavidin to a filter support. The myelin basic protein is a substrate for activated MAP kinase. The phosphorylation reaction is carried out for 10 min at 30° C. The extract can then be aspirated through the filter, which retains, the phosphorylated myelin basic protein. The filter is washed and counted for ³²P by liquid scintillation counting.

### Example 8

### MELANOPHORE TECHNOLOGY

Melanophores are skin cells found in lower vertebrates, such as amphibians. They contain pigmented organelles termed melanosomes. Melanophores are able to redistribute these melanosomes along a microtubule network upon G-protein coupled receptor (GPCR) activation. The result of this pigment movement is an apparent lightening or darkening of the cells. In melanophores, the decreased levels of intracellular cAMP that result from activation of a Gi-coupled receptor cause melanosomes to migrate to the center of the cell, resulting in a dramatic lightening in color. If cAMP levels are then raised, following activation of a Gs-coupled receptor, the melanosomes are re-dispersed and the cells appear dark again. The increased levels of diacylglycerol that result from activation of Gq-coupled receptors can also induce this re-dispersion. The response of the melanophores takes place within minutes of receptor activation and results in a simple, robust color change. The response can be easily detected using a conventional absorbance microplate reader or a modest video imaging system. Unlike other skin cells, the melanophores derive from the neural crest and appear to express a full complement of signaling proteins. In particular, the cells express an extremely wide range of G-proteins and so are able to functionally express almost all GPCRs.

Melanophores can be utilized to identify compounds, including natural ligands, against GPCRs. This method can be conducted by introducing test cells of a pigment cell line capable of dispersing or aggregating their pigment in response to a specific stimulus and expressing an exogenous GPCR such as mammalian GPR22 receptor. A stimulant, e.g., melatonin, sets an initial state of pigment disposition wherein the pigment is aggregated within the test cells if activation of the GPCR induces pigment dispersion. However, stimulating the cell with a stimulant to set an initial state of pigment disposition wherein the pigment is dispersed if activation of the GPCR induces pigment aggregation. The test cells are then contacted with chemical compounds, and it is determined whether the pigment disposition in the cells changed from the initial state of pigment disposition. Dispersion of pigments cells due to the candidate compound, including but not limited to a ligand, coupling to the GPCR will appear dark on a petri dish, while aggregation of pigments cells will appear light.

Materials and methods can be followed according to the disclosure of U.S. Pat. No. 5,462,856 and U.S. Pat. No. 6,051,386. These patent disclosures are herein incorporated by reference in their entirety.

The cells are plated in e.g. 96-well plates (one receptor per plate). 48 hours post-transfection, half of the cells on each plate are treated with 10 nM melatonin. Melatonin activates an endogenous Gi-coupled receptor in the melanophores and causes them to aggregate their pigment. The remaining half of the cells are transferred to serum-free medium 0.7×L-15 (Gibco). After one hour, the cells in serum-free media remain in a pigment-dispersed state while the melatonin-treated cells are in a pigment-aggregated state. At this point, the cells are treated with a dose response of a test/candidate compound. If the plated GPCRs bind to the test/candidate compound, the melanophores would be expected to undergo a color change in response to the compound. If the receptor were either a Gs or Gq coupled receptor, then the melatonin-aggregated melanophores would undergo pigment dispersion. In contrast, if the receptor was a Gi-coupled receptor, then the pigment-dispersed cells would be expected to undergo a dose-dependent pigment aggregation.

### Example 9

### Preparation of Expression-Enhanced GPR22R425 and GPR22 C425 Nucleic Acid

Several naturally occurring variants of the GPR22 polypeptide have been identified, including GPR22R425 and GPR22C425. Wild-type nucleic acid encoding GPR22R425 or GPR22C425 was subjected to nucleotide substitution in accordance with the methods of the subject invention and used in the expression assays described below in order to show enhanced expression of the encoded GPR22 polypeptide by the substituted nucleic acid.

The nucleotide sequence for the wild-type GPR22R425 coding region is provided in SEQ ID NO:1, and the encoded GPR22R425 amino acid sequence is provided in SEQ ID NO:2. The nucleotide sequence for the substituted GPR22R425 coding region providing for enhanced expression of the encoded GPR22R425 polypeptide is provided in SEQ ID NO:3, and the encoded GPR22R425 amino acid sequence is provided in SEQ ID NO:4. The amino acid sequences of SEQ ID NO:2 and SEQ ID NO:4 are identical.

The nucleotide sequence for the wild-type GPR22C425 coding region is provided in SEQ ID NO:5, and the encoded GPR22C425 amino acid sequence is provided in SEQ ID NO:6. The nucleotide sequence for the substituted GPR2C425 coding region providing for enhanced expression of the encoded GPR22C425 polypeptide is provided in SEQ ID NO:7, and the encoded GPR22C425 amino acid sequence is provided in SEQ ID NO:8. The amino acid sequences of SEQ ID NO:6 and SEQ ID NO:8 are identical.

The wild-type coding regions of GPR22R425 and GPR22C425 were used as template. The individual codons within the coding region were determined and the individual nucleotide substitutions to be made were identified. Individual nucleotide substitutions were introduced into the wild-type GPR22R425 and GPR22C425 coding regions and synthetic GPR22R425 and GPR22C425 encoding polynucleotides were produced and then sequenced. For instance, once designed, a full-length expression-enhanced GPR22 encoding nucleic acid was dissected into a series of contiguous segments up to about 150 bp in length with restriction sites engineered in between two neighboring segments. Each segment consisted of one or two sets of complementary oligonucleotides, which were designed such that upon annealing it will generate double stranded DNA fragments with ends compatible with specific restriction sites so that the annealed fragment could be cloned into a standard bacterial cloning vector. The sequence was confirmed by standard sequencing method before the neighboring segment generated similarly as described above was inserted into the restriction sites. The nucleic acid and deduced amino acid sequences for exemplary substituted GPR22 R425 nucleic acid providing for enhanced expression of the encoded GPR22R425 polypeptide and for substituted GPR22C425 nucleic acid providing for enhanced expression of the encoded GPR22C425 polypeptide were confirmed and are listed in the accompanying “Sequence Listing” appendix to this patent document as SEQ ID NO:3 and SEQ ID NO:7, respectively.

### Example 10

### Comparison of Expression-Enhanced GPR22 Nucleic Acid and Wild-Type Gpr22 Nucleic Acid by Cyclase Assay of GPR22 Receptor in Transfected HEK293 Cells

Thyroid-stimulating hormone (TSH, or thyrotropin) receptor (TSHR) causes the accumulation of intracellular cAMP on activation by its ligand TSH. An effective technique for measuring the decrease in production of cAMP corresponding to a constitutively active Gi-coupled receptor such as GPR22 is to co-transfect TSHR with the Gi-coupled receptor and to carry out the assay in the presence of TSH to raise the level of basal cAMP, whereby TSHR acts as a “signal window enhancer.” Such an approach was used here.

HEK293 cells were co-transfected with thyroid-stimulating hormone (TSH, or thyrotropin) receptor (TSHR) and either pCMV vector or pCMV containing a nucleic acid selected from the group consisting of substituted GPR22 (R425) [“sGPR22 (R425)”] nucleic acid providing for enhanced expression of the encoded GPR22R425 polypeptide (SEQ ID NO:3), wild-type GPR22 (R425) [“wtGPR22 (R425)”] nucleic acid (SEQ ID NO:1), substituted GPR22 (C425) [“sGPR22 (C425)”] nucleic acid providing for enhanced expression of the encoded GPR22C425 polypeptide (SEQ ID NO:7), and wild-type GPR22 (C425) [“wtGPR22 (C425)”] nucleic acid (SEQ ID NO:5). Transfection was carried out using Lipofectamine (Invitrogen). Forty-eight hours after transfection, the cells were stimulated with 100 nM TSH (Sigma) or left unstimulated (“Basal”) for 1 h before whole cell cAMP was determined using the Adenylyl Cyclase Flashplate Assay kit from Perkin Elmer catalog #:SMP004B], as described below. Results are presented in FIG. 5.

The transfected cells were placed into anti-cAMP antibody-coated wells that contained 100 nM TSH or vehicle. All conditions were tested in triplicate. After a 1 h incubation at room temperature to allow for stimulation of cAMP, a Detection Mix (provided in the Perkin Elmer kit) containing ¹²⁵I-cAMP was added to each well and the plate was allowed to incubate for another hour at room temperature. The wells were then aspirated to remove unbound ¹²⁵I-cAMP. Bound ¹²⁵I-cAMP was detected using a Wallac Microbeta Counter. The amount of cAMP in each sample was determined by comparison to a standard curve, obtained by placing known concentrations of cAMP in some wells on the plate.

As shown in FIG. 5, GP22 R425 and GPR22C425 encoded by the wild-type nucleic acid evidenced only weak Gi activities, whereas GPR2R425 and GPR2C425 GPR22 encoded by the substituted nucleic acid showed strong Gi activities and suppressed TSH-stimulated cAMP accumulation by about 80%. For the experiment shown in FIG. 5, the suppression of the level of intracellular cAMP accumulation by the substituted GPR22 nucleic acid was about 3.4 times that by the wild-type nucleic acid. These results evidence that the substituted nucleic acid provides for enhanced expression of the encoded GPR22 polypeptide. The polymorphism at amino acid 425 (R versus C) appeared to have no effect on Gi activities in either wild-type or substituted form.

### Example 11

### Comparison of Expression-Enhanced Gpr22 Nucleic Acid and Wild-Type GPR22 Nucleic Acid by IP₃ Assay of GPR22 Receptor in Gq(del)/Gi Co-Transfected HEK93 Cells

HEK293 cells were co-transfected with Gq(del)/Gi chimeric G protein and either pCMV or pCMV containing wild-type human GPR22 (“wtGPR22”) nucleic acid or substituted human GPR22 (“sGPR22”) nucleic acid providing for enhanced expression of the encoded GPR22 polypeptide. The GPR22 construct was cotransfected with Gq(del)/Gi chimera to convert Gi signaling to Gq signaling. Gq signaling was assessed by measuring a level of intracellular IP₃ accumulation, using the level of total inositol phosphate accumulation as a surrogate readout.

HEK293 cells were plated at a density of 3×10⁶ cells per 10 cm dish the day before transfection. The HEK293 cells were transfected with 2 μg of Gq(del)/Gi and 2 μg of either GPR22/pCMV or empty pCMV, using Lipofectamine™ 2000 (Invitrogen #11668-027). The day after transfection the transfected cells were replated into poly-L-lysine treated 12-well plate at 8×10⁵ cells per well and allow the cells to adhere for 4 to 6 hours.

To label the cells with ³H-myo-inositol, the medium was removed and cells were washed with inositol-free medium before 1 ml of inositol-free, serum-free medium ((Invitrogen/Gibco formula O₂-5092EA; DMEM containing D-glucose, L-glutamine, phenol red, and pyridoxine HCl, and without inositol, sodium bicarbonate, and sodium pyruvate) supplemented with 1.5 g/L sodium bicarbonate and 0.5 μCi of ³H-myo-inositol (Perkin Elmer Life Sciences) were added to each well and the cells were incubated for 16-18 hrs.

The HEK293 cells were used for IP₃ assay about 48 h post-transfection as described here. The medium was removed and replaced with 1 ml of assay medium (inositol-free medium as above supplemented with 10 μM pargyline and 10 mM lithium chloride), and the cells were incubated for 3 hours at 37° C. (To screen a test compound as a modulator of GPR22, the test compound would be included in this 3 h incubation.)

Following incubation, the medium was removed by aspiration and replaced with 300 ul of freshly-made, ice cold stop solution containing 1M KOH, 18 mM Na-borate, and 3.8 mM EDTA. The plates were incubated on ice for 5-10 min or until cells were lysed. The lysates were neutralized with 300 ul of freshly-made, ice cold neutralization solution containing 7.5% HCl, transferred to 2 ml-microcentrifuge tubes and extracted with 1 ml of chloroform:methanol (1:2) by vortexing for 15 sec and centrifuged at height speed for 5 min. One ml of the upper aqueous phase was passed through an anion exchange column preloaded with resin (Biorad, AG1-X8 100-200 mesh, formate form).

The column was washed with 10 ml of 5 mM myo-inositol followed by 10 ml of a solution containing 5 mM Na-borate and 60 mM Na-formate. Total inositol phosphates were then eluted with 2 ml of elution solution containing 0.1 M formic acid and 1M ammonium formate directly into scintillation vial. Ten ml of scintillation cocktail was added to the vial and the radioactive inositol phosphates determined by scintillation counting.

GPR22 encoded by the substituted nucleic acid showed much stronger activity in mediating IP₃ accumulation than did GPR22 encoded by the wild-type nucleic acid (FIG. 6). For the experiment shown in FIG. 6, the level of stimulation of IP₃ accumulation by GPR22 encoded by the substituted nucleic acid was about 830% the level of stimulation of IP₃ accumulation by GPR22 encoded by the wild-type nucleic acid. These results evidence that the substituted nucleic acid provides for enhanced expression of the encoded GPR22 polypeptide. This is consistent with the much stronger Gi activity of GPR22 encoded by the substituted nucleic acid compared to that of GPR22 encoded by the wild-type GPR22 as shown using cyclase assay in FIG. 5.

### Example 12

### Immunostaining of Transiently Transfected COS-7 Cells

COS-7 cells were transfected with pCMV (negative control) or with plasmid DNA corresponding to N-terminal hemagglutinin (HA) tagged β2-adrenergic receptor (“β2 adrenergic”; positive control), N-terminal HA tagged human GPR22 encoded by wild-type GPR22 nucleic acid (“Wild-Type GPR22 Nucleic Acid”) or N-terminal HA tagged human GPR22 encoded by substituted GPR22 nucleic acid providing for enhanced expression of the encoded GPR22 polypeptide (“Expression-Enhanced GPR22 Nucleic Acid”) and plated onto poly-D lysine coated chamber slide 24 hours after transfection. Forty eight hour after transfection, the cells were fixed in 4% paraformaldehyde for 15-20 minutes at room temperature and permeabilized with 0.1% Triton X-100 for 15 min at room temperature. The cells were then incubated with blocking buffer containing 2% BSA and 0.1% Triton X-100 at room temperature for 30 min followed by incubation with first antibody (mouse anti-HA antibody or rabbit anti-human GPR22 peptide antibody) solution for 1 hour. The cells were washed and incubated with fluorescence-labeled second antibody (FITC-labeled anti-mouse IgG or RITC-labeled anti-rabbit IgG) and DAPI (for nuclear staining) in the dark for 30 min. The cells were washed and the slides mounted for fluorescence microscopy.

As shown in FIG. 7, cells transfected with the substituted GPR22 nucleic acid showed a much higher level of GPR22 polypeptide expression than did cells transfected with the wild-type GPR22 nucleic acid, evidencing that the substituted nucleic acid provides for enhanced expression of the encoded GPR22 polypeptide. This is evident using either the anti-HA antibody (Roche Diagnostics Corporation, Indianapolis, Ind.) that detects the HA tag at the very N-terminus, or a GPR22 specific antibody that recognizes a peptide sequence at the C-terminal cytoplasmic tail of GPR22. The specificity of the GPR22C-terminus antibody was demonstrated by showing that only GPR22 transfected cells, but not vector transfected or β2 adrenergic receptor transfected cells were stained with GPR22C-terminus antibody. In addition, the co-staining experiment showed that GPR22C-terminus antibody labeled only those GPR22 transfected cells that also stained with anti-HA antibody.

### Example 13

### Expression of Expression-Enhanced Gpr22 mRNA in Transfected Cells

COS-7 cells were plated at a density of 3×10⁶ cells per 10 cm dish the day before transfection. The COS-7 cells were transfected with 4 μg of either pCMV containing expression-enhanced human GPR22 nucleic acid (“sGPR22”) or empty pCMV (“pCMV”), using Lipofectamine™ 2000 (Invitrogen #11668-027). Forty-eight hours after transfection, total RNA was harvested from the cells using Trizol reagent (Invitrogen) according to manufacturer's instructions. For Northern blot analysis, 20 μg total RNA was separated electrophoretically on formaldehyde containing agarose gel and transferred to PVDF membrane (Amersham). The membrane was then probed with ³²P-labeled sGPR22 cDNA fragment corresponding to nucleotides 391-935 of SEQ ID NO: 7, provided as SEQ ID NO: 22.

Hybridization was carried out overnight at 42° C. in solution containing 50% formamide, 1 M NaCl, 10% dextran sulfate, 50 mM Tris (EMD Chemicals, #9230) (pH 7.5), 1% sodium dodecyl sulfate (SDS), and 100 μg/ml denatured salmon sperm DNA. The membrane was then subjected to a series of washes, including a final wash at 65° C. with 0.2×SSC/0.1% SDS. The washed membrane was exposed to film for 25 min at room temperature. Results obtained are shown in FIG. 8.

From FIG. 8, it is apparent that sGPR2 mRNA was readily detectable after a short exposure at room temperature. Analogous Northern blot analysis carried out for wild-type GPR22 nucleic acid sequence, using suitable probe generated from wild-type GPR22 cDNA, reproducibly gave no detectable signal under equivalent exposure conditions but rather only after prolonged exposure (not shown). Without wishing to be bound by any particular theory, this difference in steady-state mRNA level is consistent with sGPR22 mRNA being more stable than wild-type GPR22 mRNA.

### Example 14

### Radiolabeled Compounds

The present invention also relates to radioisotope-labeled versions of test ligands that are useful for detecting a ligand bound to GPR22 receptor. In some embodiments, the present invention expressly contemplates a library of said radiolabeled test ligands useful for detecting a ligand bound to GPR22 receptor. In certain embodiments, said library comprises at least about 10, at least about 10², at least about 10³, at least about 10⁵, or at least about 10⁶ said radiolabeled test compounds. It is a further object of this invention to develop novel GPR22 receptor assays which comprise such radioisotope-labeled test ligands.

The invention further relates to a radioisotope-labeled version of a known ligand of GPR22 receptor for use in methods of competitive binding for identifying a candidate compound as a ligand of a GPR22 receptor.

In some embodiments, a radioisotope-labeled version of a compound is identical to the compound, but for the fact that one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (deuterium), ³H (tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compound will depend on the specific application of that radio-labeled compound. For example, for in vitro GPR22 receptor labeling and competition assays, compounds that incorporate ³H, ¹⁴C, ⁸²Br, ¹²⁵I, ¹³¹I, ³⁵S or will generally be most useful. For radio-imaging applications 11C, ¹⁸F, ¹²⁵I, ¹²³I, ¹²⁴I, ¹³¹I, ⁷⁵Br, ⁷⁶Br or ⁷⁷Br will generally be most useful. In some embodiments, the radionuclide is selected from the group consisting of ³H, C, ¹⁸F, ¹⁴C, ¹²⁵I, ¹²⁴I, ¹³¹I, ³⁵S and ⁸²Br.

Synthetic methods for incorporating radio-isotopes into organic compounds are applicable to compounds of the invention and are well known in the art. These synthetic methods, for example, incorporating activity levels of tritium into target molecules, are as follows:

A. Catalytic Reduction with Tritium Gas—This procedure normally yields high specific activity products and requires halogenated or unsaturated precursors.

B. Reduction with Sodium Borohydride [³H]—This procedure is rather inexpensive and requires precursors containing reducible functional groups such as aldehydes, ketones, lactones, esters, and the like.

C. Reduction with Lithium Aluminum Hydride [³H]—This procedure offers products at almost theoretical specific activities. It also requires precursors containing reducible functional groups such as aldehydes, ketones, lactones, esters, and the like.

D. Tritium Gas Exposure Labeling—This procedure involves exposing precursors containing exchangeable protons to tritium gas in the presence of a suitable catalyst.

E. N-Methylation using Methyl Iodide [³H]—This procedure is usually employed to prepare O-methyl or N-methyl (3H) products by treating appropriate precursors with high specific activity methyl iodide (3H). This method in general allows for higher specific activity, such as for example, about 70-90 Ci/mmol.

Synthetic methods for incorporating activity levels of ¹²⁵I into target molecules include:

A. Sandmeyer and like reactions—This procedure transforms an aryl or heteroaryl amine into a diazonium salt, such as a tetrafluoroborate salt, and subsequently to ¹²⁵I labeled compound using Na¹²⁵I. A represented procedure was reported by Zhu, D.-G. and co-workers in J. Org. Chem. 2002, 67, 943-948.

B. Ortho ¹²⁵Iodination of phenols—This procedure allows for the incorporation of ¹²⁵I at the ortho position of a phenol as reported by Collier, T. L. and co-workers in J. Labeled Compd Radiopharm. 1999, 42, S264-S266.

C. Aryl and heteroaryl bromide exchange with 125]—This method is generally a two step process. The first step is the conversion of the aryl or heteroaryl bromide to the corresponding tri-alkyltin intermediate using for example, a Pd catalyzed reaction [i.e. Pd(Ph₃P)₄] or through an aryl or heteroaryl lithium, in the presence of a tri-alkyltinhalide or hexaalkylditin [e.g., (CH₃)₃SnSn(CH₃)₃]. A represented procedure was reported by Bas, M.-D. and co-workers in J. Labeled Compd Radiopharm. 2001, 44, S280-S282.

In some embodiments, a radioisotope-labeled version of a compound is identical to the compound, but for the addition of one or more substituents comprising a radionuclide. In some further embodiments, the compound is a polypeptide. In some further embodiments, the compound is an antibody or an antigen-binding fragment thereof. In some further embodiments, said antibody is monoclonal. Suitable said radionuclide includes but is not limited to ²H (deuterium), ³H (tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compound will depend on the specific application of that radio-labeled compound. For example, for in vitro GPR22 receptor labeling and competition assays, compounds that incorporate 3H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful. In some embodiments, the radionuclide is selected from the group consisting of 3H, ¹¹C, ¹⁸F, ¹⁴C, ¹²⁵I, ¹²⁴I, ¹³¹I, ³⁵S and ⁸²Br.

Methods for adding one or more substituents comprising a radionuclide are within the purview of the skilled artisan and include, but are not limited to, addition of radioisotopic iodine by enzymatic method [Marchalonic J J, Biochemical Journal (1969) 113:299-305; Thorell J I and Johansson B G, Biochimica et Biophysica Acta (1969) 251:363-9; the disclosure of each of which is herein incorporated by reference in its entirety] and or by Chloramine-T/Iodogen/Iodobead methods [Hunter W M and Greenwood F C, Nature (1962) 194:495-6; Greenwood F C et al., Biochemical Journal (1963) 89:114-23; the disclosure of each of which is herein incorporated by reference in its entirety].

### Example 15

### Yeast Reporter Assay for GPR22 Agonist Activity

The yeast cell-based reporter assays have previously been described in the literature (e.g., see Miret et al, J Biol Chem (2002) 277:6881-6887; Campbell et al, Bioorg Med Chem Lett (1999) 9:2413-2418; King et al, Science (1990) 250:121-123; WO 99/14344; WO 00/12704; and U.S. Pat. No. 6,100,042). Briefly, yeast cells have been engineered such that the endogenous yeast G-alpha (GPA1) has been deleted and replaced with G-protein chimeras constructed using multiple techniques. Additionally, the endogenous yeast alpha-cell GPCR, Step 3 has been deleted to allow for a homologous expression of a mammalian GPCR of choice. In the yeast, elements of the pheromone signaling transduction pathway, which are conserved in eukaryotic cells (for example, the mitogen-activated protein kinase pathway), drive the expression of Fus1. By placing β-galactosidase (LacZ) under the control of the Fus1 promoter (Fus1p), a system has been developed whereby receptor activation leads to an enzymatic readout.

Yeast cells are transformed by an adaptation of the lithium acetate method described by Agatep et al (Agatep et al, 1998, Transformation of Saccharomyces cerevisiae by the lithium acetate/single-stranded carrier DNA/polyethylene glycol (LiAc/ss-DNA/PEG) protocol. Technical Tips Online, Trends Journals, Elsevier). Briefly, yeast cells are grown overnight on yeast tryptone plates (YT). Carrier single-stranded DNA (10 μg), 2 μg of each of two Fus1p-LacZ reporter plasmids (one with URA selection marker and one with TRP), 2 μg of GPR22 (e.g., human receptor) in yeast expression vector (2 μg origin of replication) and a lithium acetate/polyethylene glycol/TE buffer is pipetted into an Eppendorf tube. The yeast expression plasmid containing the receptor/no receptor control has a LEU marker. Yeast cells are inoculated into this mixture and the reaction proceeds at 30° C. for 60 min. The yeast cells are then heat-shocked at 42° C. for 15 min. The cells are then washed and spread on selection plates. The selection plates are synthetic defined yeast media minus LEU, URA and TRP (SD-LUT). After incubating at 30° C. for 2-3 days, colonies that grow on the selection plates are then tested in the LacZ assay.

In order to perform fluorimetric enzyme assays for β-galactosidase, yeast cells carrying the subject GPR22 receptor are grown overnight in liquid SD-LUT medium to an unsaturated concentration (i.e. the cells are still dividing and have not yet reached stationary phase). They are diluted in fresh medium to an optimal assay concentration and 90 μl of yeast cells are added to 96-well black polystyrene plates (Costar). Test compounds, dissolved in DMSO and diluted in a 10% DMSO solution to 10× concentration, are added to the plates and the plates placed at 30° C. for 4 h. Afler 4 h, the substrate for the β-galactosidase is added to each well. In these experiments, Fluorescein di (β-D-galactopyranoside) is used (FDG), a substrate for the enzyme that releases fluorescein, allowing a fluorimetric read-out. 20 μl per well of 500 μM FDG/2.5% Triton X100 is added (the detergent is necessary to render the cells permeable). After incubation of the cells with the substrate for 60 min, 20 μl per well of 1M sodium carbonate is added to terminate the reaction and enhance the fluorescent signal. The plates are then read in a fluorimeter at 485/535 nm.

An increase in fluorescent signal in GPR22-transformed yeast cells over that in yeast cells transformed with empty vector is indicative of a test compound being a compound that stimulates GPR22 receptor functionality. In certain embodiments, compounds of the invention give an increase in fluorescent signal above that of the background signal (the signal obtained in the presence of vehicle alone).

### Example 16

### Receptor Binding Assay

A test compound can be evaluated for its ability to reduce formation of the complex between a compound known to be a ligand of a G protein-coupled receptor of the invention and the receptor. In certain embodiments, the known ligand is radiolabeled. The radiolabeled known ligand can be used in a screening assay to identify/evaluate compounds. In general terms, a newly synthesized or identified compound (I.e., test compound) can be evaluated for its ability to reduce binding of the radiolabeled known ligand to the receptor, by its ability to reduce formation of the complex between the radiolabeled known ligand and the receptor.

In other aspect, a test compound can be radiolabeled and shown to be a ligand of a subject GPCR of the invention by evaluating its ability to bind to a cell comprising the subject GPCR or to membrane comprising the subject GPCR.

A level of specific binding of the radiolabeled known ligand in the presence of the test compound less than a level of specific binding of the radiolabeled known ligand in the absence of the test compound is indicative of less of the complex between said radiolabeled known ligand and said receptor being formed in the presence of the test compound than in the absence of the test compound.

Assay Protocol for Detecting the Complex Between a Compound Known to be a Ligand of a G Protein-Coupled Receptor of the Invention and the Receptor

A. Preparation of the Receptor

293 cells are transiently transfected with 10 ug expression vector comprising a polynucleotide encoding a G protein-coupled receptor of the invention using 60 ul Lipofectamine (per 15-cm dish). The transiently transfected cells are grown in the dish for 24 hours (75% confluency) with a media change and removed with 10 ml/dish of Hepes-EDTA buffer (20 mM Hepes+10mM EDTA, pH 7.4). The cells are then centrifuged in a Beckman Coulter centrifuge for 20 minutes, 17,000 rpm (JA-25.50 rotor). Subsequently, the pellet is resuspended in 20 mM Hepes+1 mM EDTA, pH 7.4 and homogenized with a 50-ml Dounce homogenizer and again centrifuged. After removing the supernatant, the pellets are stored at −80° C., until used in binding assay. When used in the assay, membranes are thawed on ice for 20 minutes and then 10 mL of incubation buffer (20 mM Hepes, 1 mM MgCl₂, 100 mM NaCl, pH 7.4) added. The membranes are then vortexed to resuspend the crude membrane pellet and homogenized with a Brinkmann PT-3100 Polytron homogenizer for 15 seconds at setting 6. The concentration of membrane protein is determined using the BRL Bradford protein assay.

B. Binding Assay

For total binding, a total volume of 50 ul of appropriately diluted membranes (diluted in assay buffer containing 50 mM Tris HCl (pH 7.4), 10mM MgCl₂, and 1 mM EDTA; 5-50 ug protein) is added to 96-well polyproylene microtiter plates followed by addition of 100 ul of assay buffer and 50 ul of a radiolabeled known ligand. For nonspecific binding, 50 ul of assay buffer is added instead of 100 ul and an additional 50 ul of 10 uM said known ligand which is not radiolabeled is added before 50 ul of said radiolabeled known ligand is added. Plates are then incubated at room temperature for 60-120 minutes. The binding reaction is terminated by filtering assay plates through a Microplate Devices GF/C Unifilter filtration plate with a Brandell 96-well plate harvestor followed by washing with cold 50 mM Tris HCl, pH 7.4 containing 0.9% NaCl. Then, the bottom of the filtration plate are sealed, 50 ul of Optiphase Supermix is added to each well, the top of the plates are sealed, and plates are counted in a Trilux MicroBeta scintillation counter. For determining whether less of the complex between said radiolabeled known ligand and said receptor is formed in the presence of a test compound, instead of adding 100 ul of assay buffer, 100 ul of appropriately diluted said test compound is added to appropriate wells followed by addition of 50 ul of said radiolabled known ligand.

### Example 17

### Promotion of Cardiomyocyte Survival

A subject mammalian GPR22 receptor of the invention can be shown to promote (to increase) cardiomyocyte survival as described here. More particularly, a subject mammalian GPR22 receptor of the invention can be shown to promote (to increase) cardiomyocyte survival in serum-free media as described here.

Neonatal rat ventricular myocytes (NRVMs) are prepared as described by Adams et al (J Biol Chem (1996) 271:1179-1186; the disclosure of which is herein incorporated by reference in its entirety). Briefly, hearts are obtained from 1- to 2-day old Sprague-Dawley rat pups and digested with collagenase, and myocytes are purified by passage through a Percoll gradient. The cells are cultured on laminin-coated (3.5 mg/cm²) chamber slides (Nunc) overnight in the presence of serum, washed and incubated for a further 8 hours in serum-free media DMEM/F12 (Sigma) before adenovirus infection.

Infection of NRVMs with adenovirus expression vector is carried out as described by Adams et al (Circ Res (2000) 87:1180-1187; the disclosure of which is herein incorporated by reference in its entirety). Polynucleotide encoding the mammalian GPR22 receptor is subcloned into pShuttleCMV (Qbiogene) prior to generation of recombinant GPR22 adenovirus (AdGPR22). NRVMs are infected at a multiplicity of 100 PFU/cell for 48 hours in serum-free media with AdGPR22 or with control empty adenovirus vector not containing GPR22 polynucleotide.

Cell survival is assessed at 48 hours by co-staining the NRVMs with Texas Red conjugated phalloidin and Hoechst 33342. The mammalian GPR22 receptor can be shown to promote (to increase) cardiomyocyte survival by comparing results obtained for the AdGPR22 group with those obtained for the control adenovirus group or for uninfected cells.

### Example 18

### Rescue of Cardiomyocytes from Apoptosis

A subject mammalian GPR22 receptor of the invention can be shown to rescue cardiomyocytes from apoptosis (to decrease cardiomyocyte apoptosis) as described here. More particularly, a subject mammalian GPR22 receptor of the invention can be shown to rescue cardiomyocytes from apoptosis induced by serum deprivation or from the increased apoptosis stimulated by reoxygenation (24 hours) following hypoxia (8 hours), as described here.

Neonatal rat ventricular myocytes (NRVMs) are prepared as described by Adams et al (J Biol Chem (1996) 271:1179-1186; the disclosure of which is herein incorporated by reference in its entirety). Also see Example 17, supra.

Infection of NRVMs with adenovirus expression vector is carried out as described by Adams et al (Circ Res (2000) 87:1180-1187; the disclosure of which is herein incorporated by reference in its entirety). Also see Example 17, supra. Recombinant GPR22 adenovirus (AdGPR22) and recombinant green fluorescent protein adenovirus (AdGFP) are generated. A first group of NRVMs is infected with AdGPR22. A second, control group of NRVMs is infected with AdGFP.

At 16 hours of culture in serum-free media DMEM/F12 post-infection, half of the AdGPR22-infected NRVMs and half of the AdGFP-infected NRVMs are subjected to hypoxia treatment for 8 hours. Hypoxia is achieved using an airtight incubator infused with 95% N₂ and 5% CO₂ (Van Heugten et al., J Mol Cell Cardiol (1994) 26:1513-24, the disclosure of which is herein incorporated by reference in its entirety). After hypoxia treatment, the cells are removed to ambient air and the serum-free media is refreshed.

At 48 hours post-infection, the cells are harvested. Apoptosis is assessed by analysis of oligonucleosomal DNA fragmentation (aka laddering). DNA is isolated from NRVMs using the PUREGENE DNA isolation kit according to manufacturer's instructions (Gentra). Equal amounts of DNA are separated on a 2% agarose gel, and fragmentation is detected by staining with ethidium bromide under ultraviolet light.

The mammalian GPR22 receptor can be shown to rescue cardiomyocytes from apoptosis induced by serum deprivation by comparing results obtained for the AdGPR22-infected cells not subjected to hypoxia treatment with results obtained for the control AdGFP-infected cells not subjected to hypoxia treatment.

The mammalian GPR22 receptor can be shown to rescue cardiomyocytes from the increased apoptosis stimulated by reoxygenation (24 hours) following hypoxia (8 hours) by comparing results obtained for the AdGPR22-infected cells subjected to hypoxia treatment with results obtained for the control AdGFP-infected cells subjected to hypoxia treatment.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1-74. (canceled)

75. A method comprising:
(a) identifying a codon in the coding region of a first nucleic acid that encodes a GPR22 polypeptide, wherein said codon can undergo a nucleotide substitution without changing the amino acid specified by said codon; and
(b) substituting a target nucleotide in said codon with another nucleotide to generate a synthetic nucleic acid encoding the GPR22 polypeptide, wherein said substituting increases the G/C content of said codon and does not change the amino acid specified by the codon; and
(c) synthesizing said synthetic nucleic acid;
wherein said synthetic nucleic acid, relative to said first nucleic acid, provides for increased steady state mRNA levels and enhanced expression of said GPR22 polypeptide in a eukaryotic host cell.

76. The method of claim 75, wherein the said GPR22 polypeptide is a mammalian GPR22 polypeptide.

77. The method of claim 75, wherein the eukaryotic host cell is a mammalian cell.

78. The method of claim 75, wherein the eukaryotic host cell is a melanophore or yeast cell.

79. The method of claim 75, wherein steps a and b are repeated so that at least 10% of codons have at least one target nucleotide substituted.

80. The method of claim 75, wherein steps a and b are repeated for every codon in said coding region.

81. The method of claim 75, wherein the target nucleotide is an adenine or a thymine.

82. The method of claim 81, wherein said target nucleotide is substituted with a guanine or a cytosine.

83. The method of claim 81, wherein said target nucleotide is one of at least three or more contiguous adenines or thymines within said coding region.

84. The method of claim 83, wherein said target nucleotide is substituted with a guanine or a cytosine.

85. The method of claim 75, further comprising comparing the level of expression of GPR22 encoded by said synthetic nucleic acid in a first host cell with a level of expression of GPR22 encoded by said first nucleic acid in a second host cell.

86. The method of claim 85, wherein said comparing comprises measuring receptor functionality.

87. The method of claim 86, wherein said comparing comprises measuring a GPR22 protein expression levels.

88. The method of claim 86, wherein said comparing comprises measuring GPR22 mRNA levels.

89. An isolated polynucleotide comprising a synthetic nucleic acid according to claim 75.

90. The isolated polynucleotide of claim 89, wherein said synthetic nucleic acid has a G/C content of at least 50%.

91. A vector comprising the polynucleotide of claim 89.

92. The vector of claim 91, wherein said vector is an expression vector and wherein the polynucleotide is operably linked to a promoter.

93. A recombinant host cell comprising the vector of claim 91.

94. A method for producing a GPR22 polypeptide comprising:
(a) culturing a host cell of claim 93 under conditions sufficient to express the GPR22 polypeptide from the expression vector.

95. A screening method comprising:
(a) contacting the candidate compound with a recombinant host cell of claim 93 or isolated membrane thereof; and
(b) measuring the ability of the compound to inhibit or stimulate said GPR22 polypeptide.

96. The method of claim 95, wherein said method comprises determining if said compound is a ligand of said receptor.

97. A transgenic non-human mammal comprising a polynucleotide of claim 89.

98. A method comprising:
administering a candidate compound to a transgenic non-human mammal of claim 97; and
evaluating said candidate compound for a cardioprotective activity using said non-human mammal.
